(19) United States
(12) Patent Application Publication
(10) Pub. No.: US 2022/0313629 A1
(43) Pub. Date: Oct. 6, 2022

(54) TRANSIENT POTENTIAL MELASTATIN 8 (TRPM8) ANTAGONISTS AND RELATED METHODS

(71) Applicant: MARSHALL UNIVERSITY RESEARCH CORPORATION, Huntington, WV (US)

(72) Inventor: Velvet Blair Journigan, Huntington, WV (US)

(21) Appl. No.: 17/427,456

(22) PCT Filed: Jan. 31, 2020
(86) PCT No.: PCT/US20/16200

§ 371 Date: Jul. 30, 2021

Related U.S. Application Data
(60) Provisional application No. 62/800,114, filed on Feb. 1, 2019.
(60) Provisional application No. 62/800,143, filed on Feb. 1, 2019.
(60) Provisional application No. 62/947,742, filed on Dec. 13, 2019.

(51) Int. Cl. A61K 31/166; A61P 25/02

(52) U.S. Cl. CPC A61K 31/166; A61P 25/02

(57) ABSTRACT

A TRPM8 antagonist is provided that comprises the following the formula (I) described herein. In the formula (I), R₁ is selected from a cycloalkyl, a bicycloalkyl, or a tricycloalkyl group. Each R₁ group has 5 to 12 carbon atoms. Further, each R₁ group is optionally substituted with an alkyl group having 1 to 5 carbons atoms or with a cycloalkyl group having 4 to 12 carbon atoms, and each R₁ group is optionally saturated or partially unsaturated. Methods for treating pain are further provided and comprise administering to a subject in need thereof an effective amount of a TRPM8 antagonist comprising the formula (I).

### RELATED APPLICATIONS

This application claims priority from U.S. Provisional Application Ser. No. 62/800,114, filed Feb. 1, 2019, U.S. Provisional Application Ser. No. 62/800,143, filed Feb. 1, 2019, and U.S. Provisional Application Ser. No. 62/947,742, filed Dec. 13, 2019, the entire disclosures of each of which are incorporated herein by this reference.

### TECHNICAL FIELD

The presently-disclosed subject matter generally relates to transient receptor potential melastatin 8 (TRPM8) antagonists and related methods. In particular, certain embodiments of the presently-disclosed subject matter relate to TRPM8 antagonists and methods of using those antagonists for the treatment of pain.

### BACKGROUND

TRPM8, often referred to as the ‘cold receptor’, is a polymodal nociceptor of the somatosensory nervous system and a major sensor of cold nociception in humans. TRPM8 is expressed, albeit differentially compared to other temperature sensitive TRP channels TRPA1 and TRPV1, in primary sensory neurons (Aδ and C-fibers) of the dorsal root ganglia (DRG) and trigeminal ganglia (TG), as well as in some other non-neuronal tissues. Within primary sensory neurons, TRPM8 is present on both the peripheral and central nerve terminals, supporting a role in the somatosensory and central nervous systems. This cold-sensing nociceptor initiates sensory nerve impulses after activation by mildly-cool temperatures (15-25° C.), chemical agents such as (−)-menthol (1R,2S,5R)-5-methyl-2-(1-methylethyl)-cyclohexanol), the ‘super-agonist’ icilin (3-(2-hydroxyphenyl)-6-(3-nitrophenyl)-3,4-dihydropyrimidin-2(1H)-one, also known as AG 3-5), and the endogenous lipid PIP₂. Maximal stimulation of TRPM8 by menthol occurs via a ligand stoichiometry of four menthol molecules each occupying a monomer of one functional TRPM8 tetramer.

TRPM8 appears to be a potential target for multiple therapeutic interventions. TRPM8 agonists have been explored for benign prostatic hyperplasia and prostate cancer, while antagonists have spurred more interest, for cold hypersensitivity in neuropathic pain, and migraine, among others.

A number of proprietary TRPM8 antagonists, discovered largely through high throughput screening of in-house chemical libraries, are appreciated. However, the precise molecular details of how these antagonists bind to human TRPM8 and subsequently influence its gating mechanism, as well as their underlying pharmacophores, remains to be elucidated, especially in the wake of recent success with TRPM8 and TRP channel structural biology revealed through cryo-electron microscopy (cryo-EM).

In the TRPM8-mediated Ca²⁺ flux assay, well-known TRPM8 antagonists appear to manifest either modest or variable potencies. For example, quinoline-based PF-05105679 (Pfizer, Phase 1, NCT01393652) inhibits the effects of voltage- and WS-12-mediated activation at human TRPM8 with IC₅₀ values of 103±29.4 nM and 181±7.21 nM, while 5,6,7,8-tetrahydro-1,7-naphthyridine analog AMG2850 (Amgen) exhibits IC₅₀ values ranging from 7-156 nM, against menthol, icilin and cold activation at rat and human TRPM8. 2-benzyloxy-benzoic acid amide derivative AMTB (Bayer) affords weaker inhibition against icilin-mediated responses at human TRPM8, with a pIC₅₀: 6.23±0.02. To date, however, no TRPM8 antagonists have advanced to clinical use; for example, PF-05105679 does not possess a measurable therapeutic index at high doses (600 mg and 900 mg) required to achieve unbound plasma concentrations greater than its IC₅₀ value. Likewise, Phase 1 studies of nicotinic acid analog AMG 333 (Amgen, NCT02132429) were terminated for undisclosed reasons. There remains a need for introducing more TRPM8 antagonists that are potent, reasonably specific, and thereby can sustain the possibility of successfully passing through clinical trials after successful preclinical development.

Moreover, a variety of both agonist and antagonist scaffolds with wide chemical diversity have been used to decipher the role of TRPM8 in various disease states, such as orofacial and neuropathic pain, overactive bladder (OAB) and painful bladder syndrome, diseases involving thermoregulation, oral squamous cell carcinoma, nicotine addictive behaviors, and chronic obstructive pulmonary disease. In some cases, use of these ligands as tool molecules to probe the pharmacology of TRPM8 have led to conflicting data, particularly in determining its role in chronic neuropathic pain. Highly potent chemical probes based on the cognate ligand (−)-menthol with an antagonist profile, are needed to revisit the TRPM8 field from an early drug discovery perspective, which currently lacks such small molecules of this chemotype.

Recent cryo-EM structures of both apo- and agonist-bound avian TRPM8 (TRPM8_{FA}, PDB codes 6BPQ (4.1 Å), 6NR2 (4 Å) and 6NR3 (3.4 Å)), coupled with supporting in vitro and in vivo mutagenesis studies at mouse, rat, squirrel and human orthologs, point to a definitive binding site for the prototypical ligands menthol and icilin that is likely decoupled from cold sensing. Mutagenesis studies have suggested a conserved Tyr745, located in the middle of the transmembrane S1 helix of the voltage-sensor-like domain (VSLD), is crucial for both menthol binding, as determined by radioligand displacement studies using [³H]-menthol, as well as efficacy. These studies are consistent with the structure of avian TRPM8 complexed with menthol analog WS-12 (PDB 6NR2). Constructs of the isolated S1-S4 sensing domain also suggest this region to be crucial for menthol recognition, as determined by microscale thermophoresis and NMR. Other menthol-sensitive residues include Tyr1005 and Leu1009, both of which are conserved and located in the TRP helix transmembrane domain, near the cytosolic domain interface. The positional equivalent of these two residues can be seen in the WS-12 bound structure (Tyr1004 and Leu1008 in TRPM8FA). This pocket also binds icilin with similar residues, including Tyr745 and Leu1009; as well as Gly805, Asn799 and Asp802 in the S3 helix. The cryo-EM structure of icilin-bound avian TRPM8 reinforces the notion that Tyr745, a residue conserved across all TRPM subtypes, is a common TRPM8 residue for both menthol and icilin recognition, despite their different chemotypes, while Asn799 and Asp802 are necessary for Ca²⁺-dependent icilin binding, along with Gln782 and Glu785. Additional icilin-binding residues line the orthosteric site within the VSLD and TRP helix, including Phe838, Arg841, His844 and Tyr1004 (Phe839, Arg842, His845 and Tyr1005 at hTRPM8, respectively). This ligand-binding site also overlaps with a crucial voltage- and cold-sensitive residue: a conserved Arg841 in the S4 helix (Arg842 at hTRPM8), indicating that the ability of TRPM8 to function as a polysensor of ligand and voltage stimuli is derived in part from this common site, comprised of S1-S4 helices within the VSLD. Transgenic models of hibernating ground squirrels and rats implicate residues responsible for cold vs. icilin activation, scattered throughout the VSLD in the loops linking the TM helices, at position 726 (pre-S1 domain), 762 (S1-S2 loop), 819 (S3-S4 loop), and 927, 946, 947 in the loop connecting the pore helix (PH) and S6 helix. From the sequence alignments of rat, squirrel and human TRPM8 orthologs, four of the six cold-conferring residues in rats are conserved in the human ortholog (Tyr726, Ser762, Ser819 and Asn947), raising the possibility that ligand vs. cold activation in humans occurs at topographically distinct regions. On the other hand, the remaining two mismatched residues are conserved in humans and cold-tolerant hibernating ground squirrels (Ala927, His946), suggesting either that these residues do not support a cold phenotype, or contribute to a cold response via allosteric regulation.

Accordingly, in view of the foregoing studies, additional TRPM8 antagonists that are useful in the treatment of pain would be both highly desirable and beneficial.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some embodiments of the presently-disclosed subject matter, a TRPM8 antagonist is provided that comprises the formula (I):

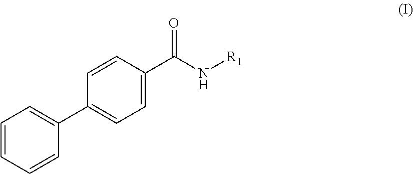

wherein R₁ is selected from a cycloalkyl, a bicycloalkyl, or a tricycloalkyl group, each of which having 5 to 12 carbon atoms, each of which optionally substituted with an alkyl group having 1 to 5 carbons atoms or with a cycloalkyl group having 4 to 12 carbon atoms, and each of which optionally saturated or partially unsaturated. In some embodiments, R₁ is a bicycloalkyl group, and the bicycloalkyl group is a fused, bridged, or spiro-connected bicycloalkyl group. In some embodiments, R₁ is a cycloalkyl group, such as, in certain embodiments, a cycloalkyl group that is a branched or substituted cycloalkyl group.

In some embodiments of the compound of Formula (I), R₁ is selected from the group consisting of

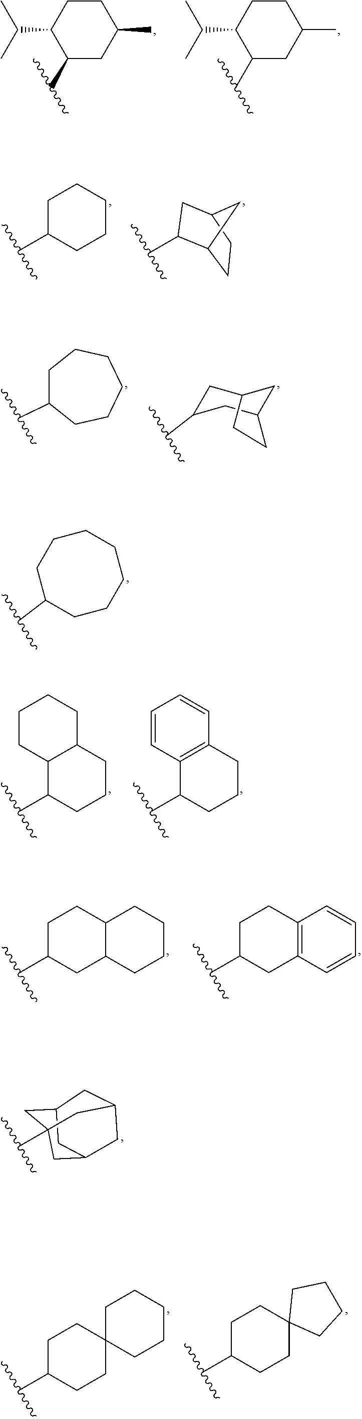

and analogs thereof.

In some embodiments, R₁ is selected from the group consisting of

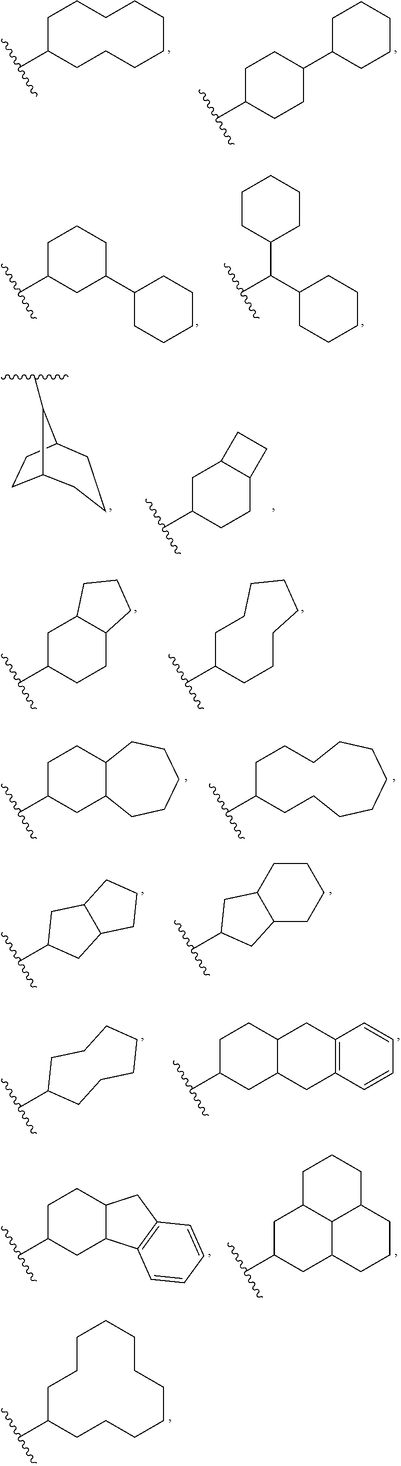

and analogs thereof.

In further embodiments, R₁ is a bicycloalkyl or tricycloalkyl group selected from the group consisting of:

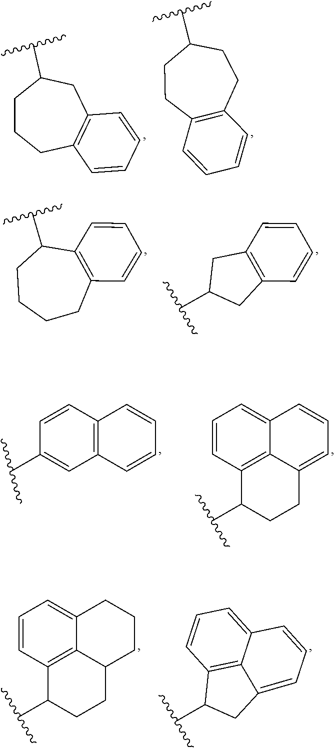

and analogs thereof.

In some embodiments, R₁ is a cycloalkyl group selected from the group consisting of:

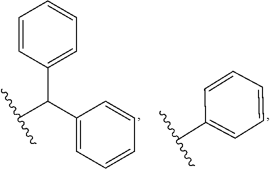

and analogs thereof.

In some embodiments, R₁ is a spiro-connected bicycloalkyl group selected from the group consisting of:

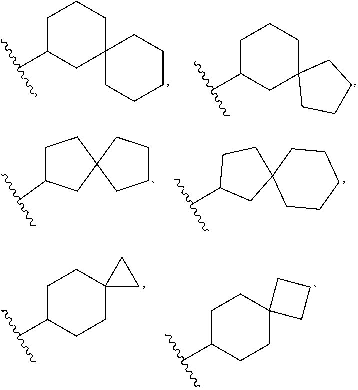

and analogs thereof.

In some embodiments, R₁ is a cycloalkyl group selected from the group consisting of:

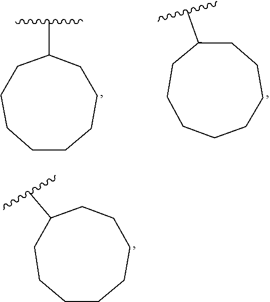

and analogs thereof.

In some embodiments, R₁ is a bicycloalkyl or tricycloalkyl group selected from the group consisting of:

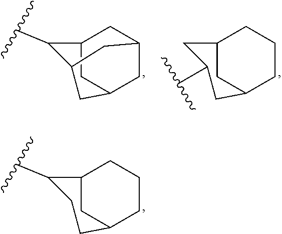

and analogs thereof.

Further provided, in some embodiments of the presently-disclosed subject matter, are pharmaceutical compositions. In some embodiments, a pharmaceutical composition is provided that comprises a TRPM8 antagonist of the presently-disclosed subject matter (e.g., a TRPM8 antagonist of Formula (I) and a pharmaceutically-acceptable vehicle, carrier, or excipient.

Still further provided, in some embodiments of the presently-disclosed subject matter are methods for treating pain. In some embodiments, a method for treating pain is provided that comprises administering to a subject in need thereof an effective amount of a TRPM8 antagonist comprising the formula (I) described herein above. In some embodiments of the therapeutic methods, the pain is neuropathic pain, such as, in certain embodiments, allodynia, chronic neuropathic pain, or chemotherapy-induced neuropathic pain. In some embodiments, administering the TRPM8 antagonist can comprise intravenously, intraperitoneally, intramuscularly, or subcutaneously injecting the TRPM8 antagonist. In some embodiments, the subject is a human.

Further features and advantages of the present invention will become evident to those of ordinary skill in the art after a study of the description, figures, and non-limiting examples in this document.

### BRIEF DESCRIPTION OF THE DRAWINGS

FIG. 1 is a chart showing reported TRPM8 antagonists, including IC₅₀ values for the antagonists as determined in the Ca²⁺ flux assay against the effect of menthol and/or icilin (icilin: 3-(2-hydroxyphenyl)-6-(3-nitrophenyl)-3,4-dihydropyrimidin-2(1H)-one);

FIGS. 2A-2D include diagrams and graphs showing convergence parameters of (−)-menthyl 1 and hTRPM8 during MD simulation, including: (FIG. 2A) a diagram showing the docking pose (pre-MD) of Compound 1 in hTRPM8, where (−)-menthyl Compound 1 is shown as magenta sticks, where important residues are shown as sticks, where individual helices are shown (1(blue), S2 (faded green), S4 (yellow green), TRP helix (red)), and where the Ca²⁺ ion (green) is shown in CPK representation; (FIG. 2B) a diagram showing the binding mode (post-MD) of Compound 1 in hTRPM8 after 100 ns MD, where (−)-menthyl Compound 1 is shown as gray sticks; (FIG. 2C) a graph showing the root-mean-square deviation (RMSD) of Compound 1 and hTRPM8 during MD simulation; and (FIG. 2D) a graph showing the energy decomposition of key residues in hTRPM8 that contributed to the binding of Compound 1; and where the hTRPM8 homology model was constructed using the cryo-EM structure of TRPM8FA (PDB 6BPQ) as a template;

FIGS. 3A-3D include diagrams and images showing convergence parameters of AMG2850 and hTRPM8 during MD simulation, including: (FIG. 3A) a diagram showing the docking pose (pre-MD) of AMG2850 in hTRPM8, where AMG2850 is shown as magenta sticks, where important residues are shown as sticks, where individual helices are shown (S2 (faded green), S3 (green), S4 (yellow-green), TRP helix (red)), and where the Ca²⁺ ion (green) is shown in CPK representation; (FIG. 3B) a diagram showing the binding mode (post-MD) of AMG2850 in hTRPM8 after 100 ns MD, where AMG2850 is shown as gray sticks; (FIG. 3C) a graph showing the RMSD of AMG2850 and hTRPM8 during MD simulation; (FIG. 3D) a graph showing the energy decomposition of key residues in hTRPM8 that contributed to the binding of AMG2850; and where the hTRPM8 homology model was constructed using the cryo-EM structure of TRPM8FA (PDB 6BPQ) as a template;

FIG. 4 is a diagram showing the superimposition of AMG2850 and (−)-menthyl Compound 1 from their MD poses (green and magentas: MD pose of TRPM8 and (−)-menthyl Compound 1, blue and gray: MD pose of TRPM8 and AMG2850);

FIG. 5 is a graph showing the evaluation of test compounds for any effect on icilin-evoked Ca²⁺ entry signals using fura-2 based Ca²⁺ imaging of HEK-293 cells stably expressing human TRPM8, where the fura-2 loaded cells bathed in Ca²⁺-free extracellular solution were exposed to icilin (500 nM) with or without the synthesized compounds or the known TRPM8 antagonist RQ-00203078 at various concentrations (for this figure, all were at 3 nM, while the corresponding histogram is provided in FIG. 15), where after approximately 4 min of compound addition, Ca²⁺ free bath solution was replaced by the one containing 2 mM Ca²⁺, where the Ca²⁺ influx was monitored as fura-2 fluorescence ratio (F355/F380), and where each data point represents mean±SEM (n≥35 cells from 3 independent experiments done in three different days);

FIGS. 6A-6B include images showing the binding mode of Compound 5 (FIG. 6A) and Compound 7 (FIG. 6B) in hTRPM8, where Compounds 5 and 7 are shown as yellow and orange sticks, respectively, where important residues are shown as sticks, where individual helices are shown (Si(blue), S2 (faded green), S4 (yellow green) and TRP helix (red)), where the Ca²⁺ ion (green) is shown in CPK representation, and where the hTRPM8 homology model was constructed using the cryo-electron microscopy (cryo-EM) structure of TRPM8FA (PDB 6BPQ) as a template;

FIGS. 7A-7B include graphs showing the inhibition of menthol-evoked TRPM8 currents by Compound 14, where currents were measured by whole-cell patch-clamp electrophysiology of HEK-293 cells transiently transfected with human TRPM8, and including: (FIG. 7A) a graph showing average current traces (n=4) measured by a +80 mV voltage pulse upon exposure to a saturating concentration of 500 μM menthol and varying concentrations of Compound 14; and (FIG. 7B) a graph showing a dose response of Compound 14 measured at concentrations from 1 nM to 1 μM at +80 mV in the presence of 500 μM menthol, where current response was normalized to the maximum current magnitude measured without antagonist for each cell, where data was fit with a single binding site Hill equation and the IC₅₀ was calculated to be 64±2 nM (n=6 cells), and where error bars represent standard error of the mean;

FIGS. 8A-8D include diagrams and graphs showing convergence parameters of 14 and hTRPM8 during MD simulation, including: (FIG. 8A) a graph showing the binding mode (pre-MD) of Compound 14 in hTRPM8, where Compound 14 is shown as magenta sticks, where important residues are shown as sticks, where individual helices are shown (Si (blue), S2 (faded green), S3 (green), S4 (yellow-green), TRP helix (red)), and where the Ca²⁺ ion (green) is shown in CPK representation; (FIG. 8B) a diagram showing the binding mode (post-MD) of Compound 14 in hTRPM8; (FIG. 8C) a graph showing the RMSD of Compound 14 and hTRPM8; and (FIG. 8D) a graph showing the energy decomposition of key residues in hTRPM8 that contributed to the binding of Compound 14, where the hTRPM8 homology model was constructed using the cryo-EM structure of TRPM8FA (PDB 6BPQ) as a template;

FIG. 9 includes related diagrams showing the binding pocket of Compound 14 in TRPM8, TRPA1 and TRPV1 and their binding mode, including: (panel (a)) a diagram showing the position of the binding pocket of Compound 14 in TRPM8, TRPA1 and TRPV1; (panel (b)); a diagram showing the binding mode of Compound 14 in hTRPA1 (PDB 3J9P); (panel (c)) a diagram showing the binding mode of Compound 14 in hTRPM8, where the hTRPM8 homology model was constructed using the cryo-electron microscopy (cryo-EM) structure of TRPM8FA (PDB 6BPQ) as a template; and (panel (d)) a diagram showing the binding mode of Compound 14 in hTRPV1 (PDB 3J5R);

FIG. 10 is a graph showing the effect of Compound 14 on icilin-induced wet-dog shakes (WDS) in ICR (CD1) mice, where compound or vehicle was administered 30 minutes before icilin injection, where gabapentin (25 mg/kg) was administered 1h before icilin injection, where after i.p. injection of icilin (10 mg/kg), the number of WDS were counted over 30 min, and where data are given as mean±SEM (n=6) (2-way ANOVA with Bonferroni post hoc test, **P<0.01);

FIG. 11 is a graph showing the effect of Compound 14 on Oxaliplatin (OXP)-induced cold allodynia in male C57-mice, where mice were given three i.p. injection of OXP (6 mg/kg) or the vehicle (saline and a 5% Mannitol solution, black line (control)) on alternate days, where the day 7 after administration, cold allodynia was evaluated by the acetone test, where time-course of cold allodynia without compound injection (green line) or injection of Compound 14 at 0.1 μg (blue line) or at 1.0 μg (red line) are shown, and where data are given as mean±SEM n=6 (one-way ANOVA with Dunnett's post hoc test. *P<0.05; ***P<0.001****P<0.0001);

FIG. 12 is a diagram showing sequence alignment of S1-S4 helices (VSLD), S5-S6 helices (pore domain), pore helix (PH) and TRP helix for collared flycatcher (avian), human and rat TRPM8, where the VSLD domain (S1-S4), pore domain (S5-S6, and pore helix PH) and TRP helix from full sequence of TRPM8FA (U3JD03, 1103 residues), hTRPM8 (Q7Z2W7, 1104 residues) and rTRPM8 (Q8R455, 1104 residues) was retrieved from the UniProtKB/Swiss-Prot, and the three domains were aligned using the Clustal Omega program, where similar amino acids are highlighted, where TRPM8FA is shown to be highly homologous to human and rat TRPM8 in the aforementioned region (86% and 87% sequence identity, respectively), and where astericks denote residues sensitive to menthol (green), icilin (yellow), voltage (gold) and Ca²⁺ (magenta);

FIG. 13 is a graph showing a concentration—response curve for icilin in HEK293 stably expressing hTRPM8 cells, where response was reported by peak fluorescence (Fura-2) ratio indicative of maximum changes in intracellular free Ca²⁺ concentrations at indicated concentrations of icilin, and where each data point represents peak fluorescence ratio as mean±SEM (n≥30 cells from 3 independent experiments done in three different days);

FIG. 14 is a graph showing a concentration-response curve of (−) menthol-evoked Ca²⁺ entry in HEK-293-hTRPM8 cells, where each data point represents mean response at cognate concentration expressed as a percent of the maximum Ca²⁺ entry in a Fura-2 based Ca²⁺ imaging assay, where the error bars indicate SEM (n≥35 cells from 3 independent experiments done in three different days), and where the line represents a four parameter logistic curve fit done using Prism 7 (Graphpad Inc.);

FIG. 15 includes graphs showing the evaluation of the effect of the compounds on icilin-evoked Ca²⁺ entry in HEK-293 cells stably expressing human TRPM8, where each bar represents peak Fura-2 ratio normalized to that of the icilin (500 nM) response and expressed as mean±SEM (n≥35 cells from 3 independent experiments done in three different days), and where one way ANOVA followed by Dunnett's test was used to compare the efficacy of the test compounds with respect to that of the control (icilin) (*P<0.001);

FIG. 16 is a graph showing a concentration-response curve of RQ-00203078, where the response was reported by peak fluorescence (Fura-2) ratio normalized to the peak response triggered by 500 nM icilin alone, and where each data point represents mean±SEM (n≥30 cells from 3 independent experiments done in three different days);

FIG. 17 is a graph showing a concentration-response curve of Compound 14, where response was reported by peak fluorescence (Fura-2) ratio normalized to the peak response triggered by 500 nM icilin alone, and where each data point represents mean±SEM (n≥30 cells from 3 independent experiments done in three different days);

FIG. 18A is a graph showing a typical trace presenting the effect of Compound 14 (3 nM) on (−)-menthol (100 μM)-evoked Ca²⁺ entry signals using Fura-2 based Ca²⁺ imaging of HEK-293-hTRM8 cells, where each data points represent mean±SEM (n≥35 cells from 3 independent experiments done in three different days);

FIG. 18B is a graph showing the evaluation of the effect of 3 nM of Compound 14 on (−) menthol-evoked Ca²⁺ entry in HEK-293-hTRPM8, where each bar represents peak Fura-2 ratio normalized to that of the menthol (100 μM) response and expressed as mean±SEM (n≥35 cells from 3 independent experiments done in three different days), and where the mean responses between two groups were compared using Student's t-test, 2 tailed, unpaired;

FIG. 19 is a graph showing the intrinsic hTRPM8 activity of selected biphenyl amide analogs, where Fura-2 based ratiometric Ca²⁺ imaging was performed using Ca²⁺ (2 mM) containing extracellular solution, and where each data point represents mean±SEM (n≥20 cells from 3 independent experiments done in three different days);

FIG. 20 is a graph showing agonist dose-response of AITC-activation of hTRPA1m where the EC₅₀=7.62±0.89 μM, and where each data point represents mean±SEM, n=5 or 10 independent experiments;

FIG. 21 is a graph showing ruthenium red blockade of AITC-activated (10 μM) hTRPA1 responses, where the IC₅₀=162±33 nM, and where each data point represents mean SEM, n=5 independent experiments;

FIG. 22 is a graph showing the agonist dose-response of capsaicin-activation of hTRPV1, where the EC₅₀=19±2.5 nM, and where each data point represents mean±SEM, n=5 or 10 independent experiment; and

FIG. 23 is a graph showing capsazepine blockade of capsaicin-activated (100 nM) hTRPV1 responses, where the IC₅₀=451±48 nM, and where each data point represents mean SEM, n=5 independent experiments.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, UniProt or Swiss-Prot databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended), “consist of” (closed ended), or “consist essentially of” the components of the presently-disclosed subject matter as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments±1%, in some embodiments ±0.5%, and in some embodiments±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

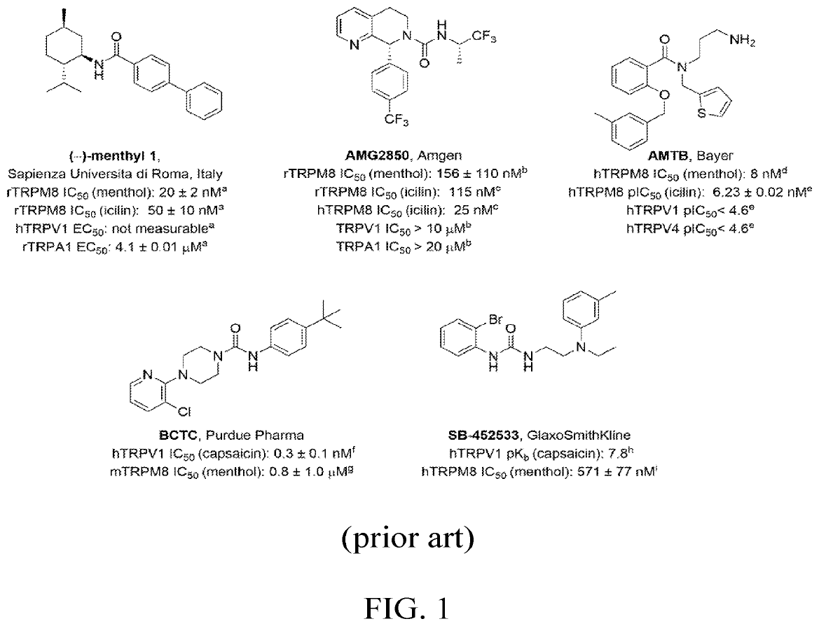

The presently-disclosed subject matter is based, at least in part, on the development of small molecule TRPM8 antagonists with both structural similarities and conformational rigidity and that were used to reveal similar molecular determinants for ligand recognition, via their poses from molecular dynamics (MD) simulations in a human TRPM8 homology model based on the avian cryo-EM structure TRPM8FA (PDB 6BPQ, ˜4.1 Å). Those insights allowed for the design of novel TRPM8 chemical probes and therapeutic compounds for TRPM8-mediated sensory neuropathies that, in certain embodiments, were identified as has having potencies greater than currently known TRPM8 antagonists such as 2-benzyloxy-benzoic acid amide derivative AMTB, as well as TRPV1 antagonists with bifunctional TRPM8 antagonist activity, such as pyridylpiperazine carboxamide BCTC and urea SB-452533 (FIG. 1).

In preclinical models, it is appreciated that a tryptophan-based TRPM8 antagonist reverses oxaliplatin-induced cold allodynia in C57/BL6 mice. In this regard, the presently-disclosed, chemically novel, biphenyl amide-based TRPM8 antagonists, with high nanomolar potency and modest TRPM8 selectivity, have been found to reverse cold allodynia in C57/BL6 mice with chemotherapy (oxaliplatin)-induced chronic neuropathic pain (which is also termed chemotherapy-induced peripheral neuropathy or CIPN). Further, the TRPM8 antagonists disclosed herein can be useful in other pain states, such as cold allodynia and mechanical allodynia associated with chronic neuropathic pain. Prior TRPM8 antagonists DFL23448 and DFL23693 significantly attenuate nociceptive responses in chronic constriction injury (CCI)-induced cold allodynia and mechanical allodynia models of neuropathic pain. Similarly, the prior TRPM8 antagonist PBMC decreases CCI-induced cold allodynia in preclinical models. In line with these observations, TRPM8^{-/-} mice do not show an increase in cooling-induced sensitivity (cold allodynia) following CCI of the sciatic nerve; and, similarly, downregulation of TRPM8 in the L5 dorsal root ganglion (DRG) ipsilateral to CCI-induced nerve injury using TRPM8 antisense oligonucleotides (intrathecal administration) results in attenuated cold hyperalgesia. Without wishing to be bound by any particular theory or mechanism, it was thus believed that the TRPM8 antagonists described herein can also alieve thermal and mechanical allodynia in a chronic neuropathic pain state, with the potential to provide novel, non-opioid therapies for the treatment of chronic pain, thus providing a new treatment for opioid addiction. In some embodiments of the present invention, TRPM8 represents a target for the TRPM8 antagonists described herein for novel pain pharmacotherapies controlling sensory responses to pain stimuli at the level of the periphery and/or CNS. In some embodiments, the presently-described TRPM8 antagonists can also find utility in other diseases, such as cold hypersensitivity in inflammatory pain, orofacial pain, overactive bladder and painful bladder syndrome, diseases involving thermoregulation including diseases where the induction of hypothermia or decreasing body temperature is beneficial (for example, during cardiac arrest, neonatal encephalopathy, stroke, and others), migraine, prostate cancer, oral squamous cell carcinoma, nicotine addictive behaviors, and others.

The presently-disclosed subject matter thus includes TRPM8 antagonists and related methods. In particular, certain embodiments of the presently-disclosed subject matter relate to TRPM8 antagonists and methods of using those antagonists for the treatment of pain.

In some embodiments of the presently-disclosed subject matter, a TRPM8 antagonist is provided having the following formula (I):

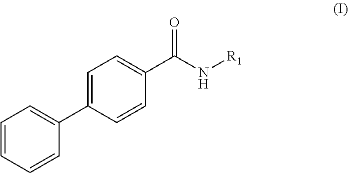

wherein R₁ is selected from a cycloalkyl, a bicycloalkyl, or a tricycloalkyl group, each of which having 4 to 12 carbon atoms, each of which optionally substituted with an alkyl group having 1 to 6 carbons atoms or with a cycloalkyl group having 4 to 12 carbon atoms, and each of which optionally saturated or partially unsaturated. As shown below, [symbol]
is used herein to indicate the point of attachment for the various R₁ groups.

The term “cycloalkyl” is used herein to generally refer to a non-aromatic monocyclic ring system of about 3 to about 12 carbon atoms, e.g., 3, 4, 5, 6, 7, 8, 9, 10, 11, or 12 carbon atoms although, in certain embodiments, such cycloalkyl groups are further inclusive of aromatic rings. Representative monocyclic cycloalkyl rings include, but are not limited to, cyclopropyl, cyclobutyl, cyclopentyl, cyclohexyl, cycloheptyl, and the like.

In some embodiments, such cycloalkyl groups can also be combined to create multicyclic ring systems such as, in some embodiments, “bicycloalkyl” groups including two ring systems or, in other embodiments, “tricycloalkyl” groups including three ring systems. In some embodiments, such bicycloalkyl groups can be fused bicycloalkyl groups, whereby the two rings are directly connected to one another and share two adjacent atoms. In other embodiments, the bicycloalkyl groups are bridged bicycloalkyl groups, whereby the two rings share three or more atoms with two bridgehead atoms shared by the two rings and separated by a bridge containing at least one atom. In other embodiments, the bicycloalkyl groups are spiro-connected bicycloalkyl groups, whereby the two rings are connected by a single atom.

In some embodiments, the cycloalkyl groups described herein can also be optionally saturated or partially unsaturated. The cycloalkyl group also can be optionally substituted with an alkyl group as defined herein or with another cycloalkyl group. In some embodiments, the cycloalkyl group is a branched cycloalkyl group whereby the cycloalkyl group is attached to another chemical moiety, such as an alkyl group.

With respect to the alkyl groups described herein and that can be used to optionally substitute the cycloalkyl, bicycloalkyl, or tricycloalkyl groups of the presently-disclosed subject matter, the term “alkyl” is used herein to refer to C1-20 inclusive, linear (i.e., “straight-chain”), branched, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, methylpropynyl, butynyl, pentynyl, hexynyl, heptynyl, and allenyl groups. “Branched” when used herein in reference to an alkyl group refers to an alkyl group in which typically a lower alkyl group, such as methyl, ethyl or propyl, is attached to a linear alkyl chain. “Lower alkyl” refers to an alkyl group having 1 to about 8 carbon atoms (i.e., a C1-8 alkyl), e.g., 1, 2, 3, 4, 5, 6, 7, or 8 carbon atoms. “Higher alkyl” refers, on the other hand, to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C1-8 straight-chain alkyls. In other embodiments, “alkyl” refers, in particular, to C1-8 branched-chain alkyls.

In some embodiments of the presently-disclosed subject matter, a compound of formula (I) above is provided where R₁ is selected from the group consisting of:

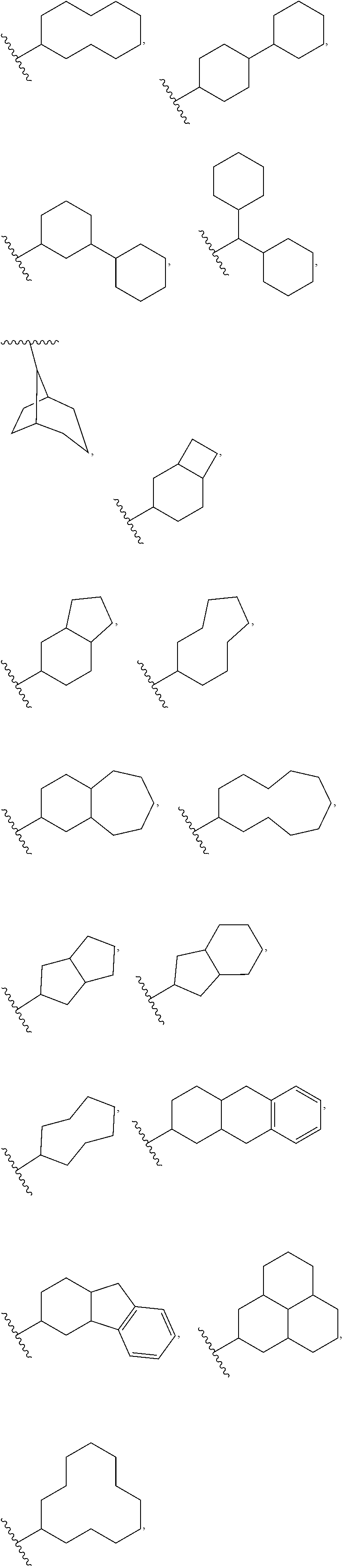

and analogs thereof.

In some embodiments, R₁ is a bicycloalkyl or tricycloalkyl group selected from the group consisting of:

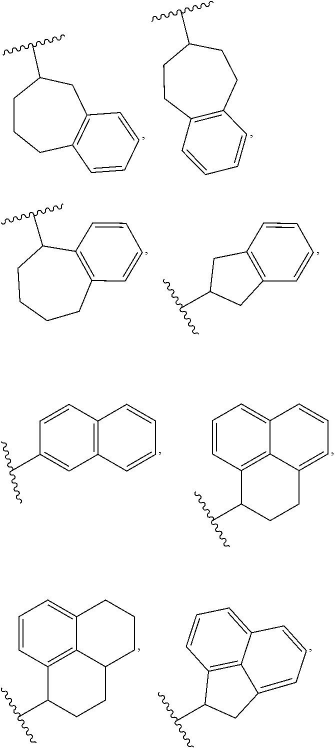

and analogs thereof.

In other embodiments, R₁ is a cycloalkyl group selected from the group consisting of:

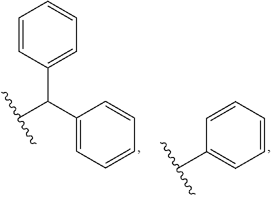

and analogs thereof.

In further embodiments, R₁ is a spiro-connected bicycloalkyl group selected from the group consisting of:

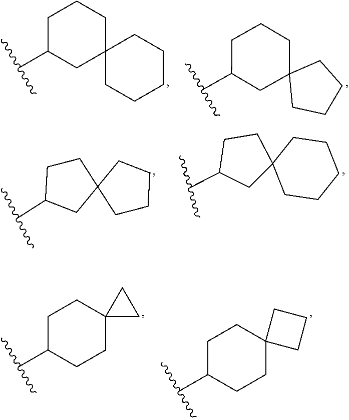

and analogs thereof.

In some embodiments, R₁ is a cycloalkyl group selected from the group consisting of:

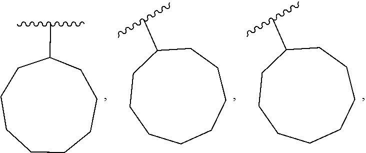

and analogs thereof.

In some embodiments, R₁ is a bicycloalkyl or tricycloalkyl group selected from the group consisting of:

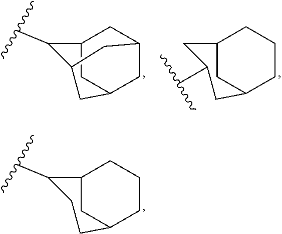

and analogs thereof.

In yet further embodiments of formula (I) of the presently-disclosed subject matter, R₁ is selected from the group consisting of:

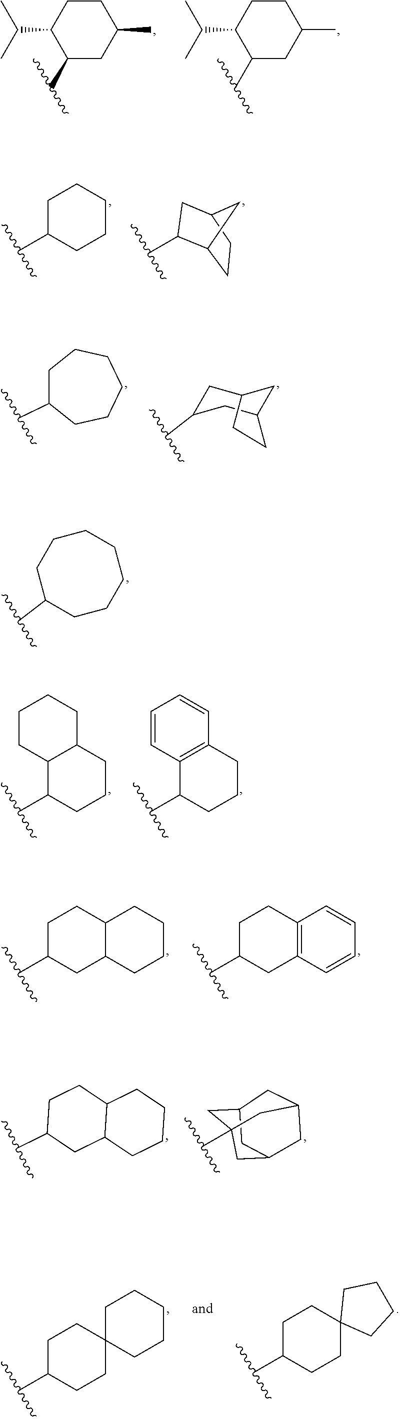

For example, in some embodiments, a TRPM8 antagonist is provided having the following formula (II), which is also referred to herein as Compound 1:

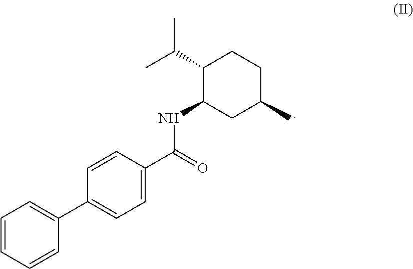 [SMILES: CC(C)[C@@H]1CC[C@@H](C)C[C@H]1NC(=O)c1ccc(-c2ccccc2)cc1]

As another example, in some embodiments, a TRPM8 antagonist is provided having the following formula (III), which is also referred to herein as Compound 2:

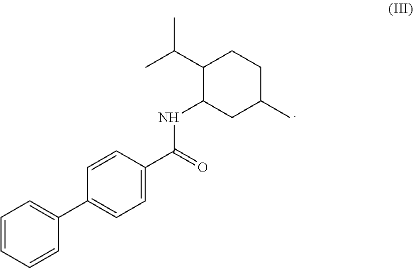 [SMILES: CC1CCC(C(C)C)C(NC(=O)c2ccc(-c3ccccc3)cc2)C1]

As another example, in some embodiments, a TRPM8 antagonist is provided having the following formula (IV), which is also referred to herein as Compound 3:

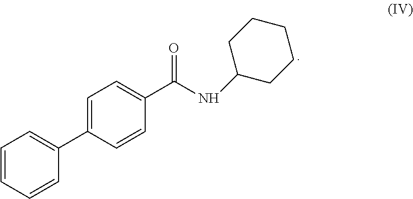 [SMILES: O=C(NC1CCCCC1)c1ccc(-c2ccccc2)cc1]

As another example, in some embodiments, a TRPM8 antagonist is provided having the following formula (V), which is also referred to herein as Compound 4:

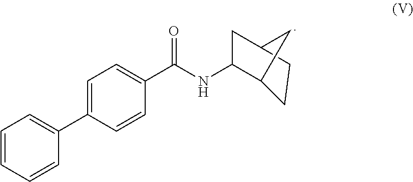 [SMILES: O=C(NC1CC2CCC1C2)c1ccc(-c2ccccc2)cc1]

As another example, in some embodiments, a TRPM8 antagonist is provided having the following formula (VI), which is also referred to herein as Compound 5:

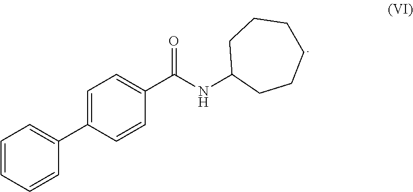 [SMILES: O=C(NC1CCCCCC1)c1ccc(-c2ccccc2)cc1]

As another example, in some embodiments, a TRPM8 antagonist is provided having the following formula (VII), which is also referred to herein as Compound 6:

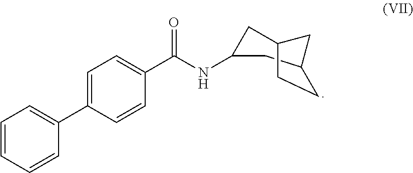 [SMILES: O=C(NC1CC2CCC(C2)C1)c1ccc(-c2ccccc2)cc1]

As another example, in some embodiments, a TRPM8 antagonist is provided having the following formula (VIII), which is also referred to herein as Compound 7:

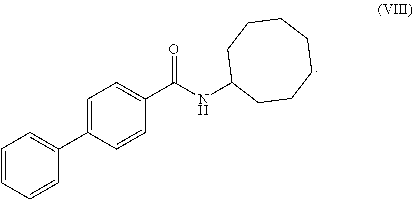 [SMILES: O=C(NC1CCCCCCC1)c1ccc(-c2ccccc2)cc1]

As another example, in some embodiments, a TRPM8 antagonist is provided having the following formula (IX), which is also referred to herein as Compound 8:

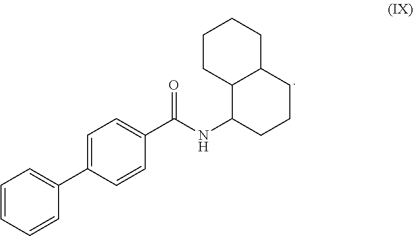 [SMILES: O=C(NC1CCCC2CCCCC21)c1ccc(-c2ccccc2)cc1]

As another example, in some embodiments, a TRPM8 antagonist is provided having the following formula (X), which is also referred to herein as Compound 9:

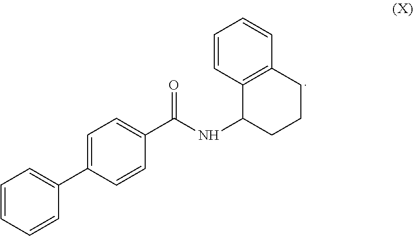 [SMILES: O=C(NC1CCCc2ccccc21)c1ccc(-c2ccccc2)cc1]

As another example, in some embodiments, a TRPM8 antagonist is provided having the following formula (XI), which is also referred to herein as Compound 10 or 12:

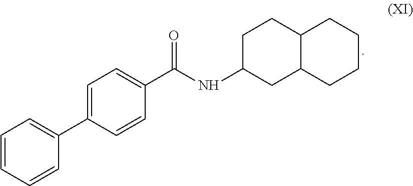 [SMILES: O=C(NC1CCC2CCCCC2C1)c1ccc(-c2ccccc2)cc1]

In some embodiments, the TRPM8 antagonist having the following formula (XI) above can be provided in a number of isomeric forms, including, but not limited to:

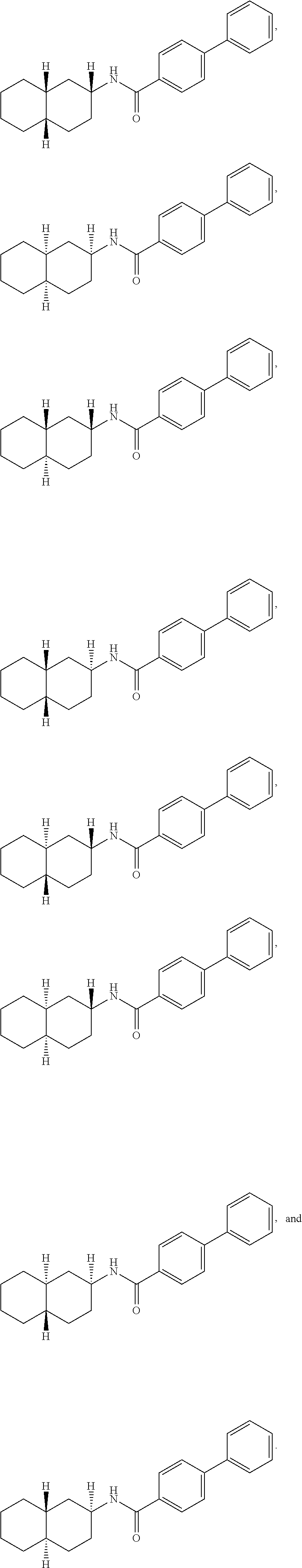

As another example, in some embodiments, a TRPM8 antagonist is provided having the following formula (XII), which is also referred to herein as Compound 11:

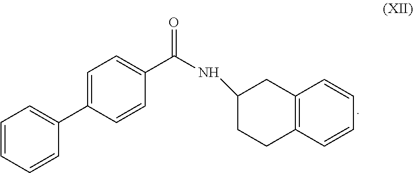 [SMILES: O=C(NC1CCc2ccccc2C1)c1ccc(-c2ccccc2)cc1]

As another example, in some embodiments, a TRPM8 antagonist is provided having the following formula (XIII), which is also referred to herein as Compound 15:

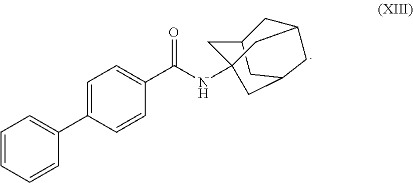 [SMILES: O=C(NC12CC3CC(CC(C3)C1)C2)c1ccc(-c2ccccc2)cc1]

As another example, in some embodiments, a TRPM8 antagonist is provided having the following formula (XIV), which is also referred to herein as Compound 13:

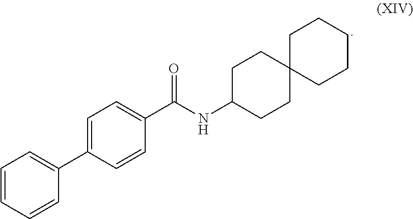 [SMILES: O=C(NC1CCC2(CCCCC2)CC1)c1ccc(-c2ccccc2)cc1]

As another example, in some embodiments, a TRPM8 antagonist is provided having the following formula (XV), which is also referred to herein as Compound 14:

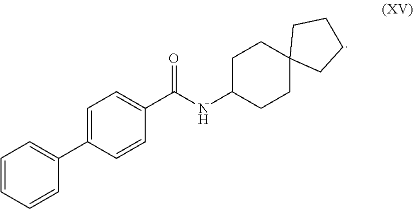 [SMILES: O=C(NC1CCC2(CCCC2)CC1)c1ccc(-c2ccccc2)cc1]

Further provided, in some embodiments of the presently-disclosed subject matter, are pharmaceutical compositions that include the TRPM8 antagonists described herein and a pharmaceutically-acceptable vehicle, carrier, or excipient. Indeed, when referring to certain embodiments herein, the terms “TRPM8 antagonist” and/or “compound” may or may not be used to refer to a pharmaceutical composition that includes the TRPM8 antagonists.

The term “pharmaceutically-acceptable carrier” as used herein refers to sterile aqueous or nonaqueous solutions, dispersions, suspensions, or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants, such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like.

Prolonged absorption of the injectable pharmaceutical form can be brought about by the inclusion of agents, such as aluminum monostearate and gelatin, which delay absorption.

Injectable depot forms are made by forming microencapsule matrices of the drug in biodegradable polymers such as polylactide-polyglycolide, poly(orthoesters) and poly(anhydrides). Depending upon the ratio of compound to biodegradable polymer and the nature of the particular biodegradable polymer employed, the rate of compound release can be controlled. Depot injectable formulations can also be prepared by entrapping the compound in liposomes or microemulsions, which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Suitable formulations can further include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents.

The compositions can also take forms such as suspensions, solutions, or emulsions in oily or aqueous vehicles, and can contain formulatory agents such as suspending, stabilizing and/or dispersing agents. Alternatively, the compounds can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated by methods known in the art.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically-acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration, the compositions can take the form of tablets or lozenges formulated in a conventional manner.

The compositions can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). The compounds can also be formulated in rectal compositions, creams or lotions, or transdermal patches.

Still further provided, in some embodiments of the presently-disclosed subject matter, are methods for treating pain utilizing the TRPM8 antagonists described herein. As used herein, the terms “treatment” or “treating” relate to any treatment of a condition of interest (e.g., pain), including but not limited to prophylactic treatment and therapeutic treatment. As such, the terms “treatment” or “treating” include, but are not limited to: reducing the occurrence of a condition of interest or the development of a condition of interest; inhibiting the progression of a condition of interest; arresting or reducing the further development of a condition of interest; reducing the severity of a condition of interest; ameliorating or relieving symptoms associated with a condition of interest; and causing a regression of a condition of interest or one or more of the symptoms associated with a condition of interest.

The term “pain” is used herein to generally describe physical suffering or discomfort caused by illness or injury, and which is typically conveyed to the brain of a subject by sensory neurons to signal actual or potential injury to the body. Such pain can arise from any number of situations, including injury and illness, but can also result from psychological conditions, such as depression, or in the absence of a recognizable trigger. Pain is inclusive of both acute pain and chronic pain. Acute pain often results from tissue damage, such as a skin burn, broken bone, headaches, and muscle cramps, and will usually go away as the injury heals or the cause of the pain (stimulus) is removed. Chronic pain, on the other hand, persists or progresses over a long period of time and is often resistant to medical treatments. Such chronic pain can arise as the result of a number of different medical conditions including, but not limited to, diabetes, arthritis, migraines, fibromyalgia, cancer, shingles, sciatica, and previous trauma or injury, and can worsen in response to environmental and/or psychological factors. Other types of chronic pain include allodynia, hyperalgesia, and phantom limb pain, as well as other types of long lasting pain that arises due to, or is exacerbated by, at least some damage to the nervous system (i.e., neuropathic pain). In some embodiments of the methods described herein, the pain treated by the presently-described TRPM8 antagonists is chronic neuropathic pain. In some embodiments, the pain being treated is chemotherapy-induced neuropathic pain, cold allodynia in chronic neuropathic pain, mechanical allodynia in chronic neuropathic pain, cold hypersensitivity in inflammatory pain and nerve injury, orofacial pain, cold-induced pain, migraine, painful bladder syndrome.

In some embodiments, each of the above-mentioned types of pains, including the severity of such types of pain, can be assessed by methods known to those skilled in the art. For instance, in some embodiments, standard clinical measurements of pain used to assess chemotherapy-(e.g., oxiplatin) induced peripheral neuropathy include the Cylinder Test, FACT/GOG-NTX-13 or FACT/GOG-NTX-4 (neurotoxicity subscales) and BPI-SF Average Pain Severity Item. In some embodiments, standard clinical measurements of neuropathic pain include the Cold Pain Test, measurement of change in average pain intensity, measurement of change from baseline in the pain intensity scores during a specified period, measurements of a change from baseline in the weekly average of the daily average pain rating, measurement of a change in mean daily pain intensity score, measurements of a reduction of pain intensity following bunionectomy, measurement of average daily pain intensity, and/or measurements using the Numeric or Numerical Pain Rating Scale (NRS or NPRS).

For administration of a therapeutic composition as disclosed herein (e.g., a TRPM8 antagonist), conventional methods of extrapolating human dosage based on doses administered to a murine animal model can be carried out using the conversion factor for converting the mouse dosage to human dosage: Dose Human per kg=Dose Mouse per kg/12 (Freireich, et al., (1966) Cancer Chemother Rep. 50: 219-244). Doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m2.

Suitable methods for administering a therapeutic composition in accordance with the methods of the presently-disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, intrathecal administration, intracerebroventricular administration, inhalation, intratracheal installation, surgical implantation, transdermal delivery, local injection, intranasal delivery, and hyper-velocity injection/bombardment. Where applicable, continuous infusion can enhance drug accumulation at a target site (see, e.g., U.S. Pat. No. 6,180,082).

Regardless of the route of administration, the compositions of the presently-disclosed subject matter are typically administered in an amount effective to achieve the desired response. As such, the term “effective amount” is used herein to refer to an amount of the therapeutic composition (e.g., the TRPM8 antagonist and a pharmaceutically-acceptable vehicle, carrier, or excipient) sufficient to produce a measurable biological response (e.g., a decrease in pain). Actual dosage levels of active ingredients in a therapeutic composition of the presently-disclosed subject matter can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated.

Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, New Jersey; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Fla.; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pa.; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The presently-disclosed subject matter is further illustrated by the following specific but non-limiting examples.

### EXAMPLES

Materials and Methods

Molecular Modeling

Homology Modeling of hTRPM8. A human TRPM8 model was built based on the cryo-EM structure of the collared flycatcher Ficedula albicollis (TRPM8FA, PDB: 6BPQ, Resolution: ˜4.1 Å), with which hTRPM8 shares 83% sequence identity to TRPM8FA. The cryo-EM structure was downloaded from the Protein Data Bank. SYBYL-X 1.3 was applied to repair all the residues and minimize the energy. The full sequence of hTRPM8 (Q7Z2W7, 1104 residues) was retrieved from the UniProtKB/Swiss-Prot. The sequence alignments and homology modeling used a previously-reported protocol.

Modeller 9.18 was used to construct the hTRPM8 model. Once the 3D models were generated, SYBYL-X 1.3 was used to conduct the energy minimizations. Briefly, the parameters defined in the SYBYL were as follows: gradient was set to 0.5 kcal/mol, max iterations were set to 5000, force field was set to MMFF94s, and the charges were set to MMFF94. All these settings were the same as described previously. Then proSA-web Z-scores and PROCHECK Ramachandran plots were used to check the overall quality of the 3D models.

Molecular docking Ligands into hTRPM8. The docking program Surflex-Dock GeomX (SFXC) implemented in SYBYL-X 1.3 was used to build TRPM8-ligand complexes, in which the total score was expressed in − log₁₀ (K_{d}). Then MOLCAD module in SYBYL-X 1.3 was applied to explore the potential binding pocket of hTRPM8, which included the residues Phe738 (Si), Tyr745 (S1), Glu782 (S2), Glu785 (S2), Trp798 (S3), Asn799 (S3), Asp802 (S3), Ala805 (S3), Arg842 (S4), His845 (S4), Ile846 (S4), Val849 (S4), Glu1004 (TRP helix), Tyr1005 (TRP helix), Arg1008 (TRP helix) and Leu1009 (TRP helix). All the parameters and the protocol of molecular docking can be found as previously-reported.

Molecular Dynamics (MD) Simulations. In order to accelerate the MD simulation, the transmembrane domain of hTRPM8 (residues from Gln671 to Asn1010) was selected to carry out the simulations of TRPM8 with different ligands. Each monomer of the TRPM8 receptor was complexed with one ligand, so there were four (same) ligands for each simulation. Then the system was solvated into a 0.15 mol/L NaCl solution, including 514 1-palmitoyl-2-oleoyl-sn-glycero-3-phosphocholine (POPC) lipids, 51470 water molecules, and 156 Cl⁻ and 140 Na²⁺ ions. The initial conformation of TRPM8 and ligands were taken from molecular docking studies. The sizes of the initial simulation boxes were set to ˜151 Å*151 Å*119 Å. The AMBER ff14SB force field was applied to TRPM8 and the AMBER Lipid14 force field was applied to lipids. Water molecules were treated with the TIP3P water model.

All of the MD simulations were then conducted using the PMEMD.mpi and PMEMD.cuda modules in the AMBER16 package. In order to avoid possible steric clashes, several minimization steps were performed for each system. Then, each system was gradually heated from 0 K to 300 K during the heating stage, followed by equilibrium and production stages (300 K) with a time step of 2 fs. A periodic boundary condition was employed to maintain constant temperature and pressure (NPT) ensembles. The pressure was set to 1 atm and controlled by the anisotropic (x−, y−, z−) pressure scaling protocol with a pressure relaxation time of 1 ps. The temperature kept constrained by Langevin dynamics with a collision frequency of 2 ps-1. Long-range electrostatics were handled by the Particle Mesh Ewald (PME) method and the cutoff value of real-space interactions was set to 10 Å. All covalent bonds involving hydrogen atoms were constrained by the SHAKE algorithm. Each system was carried out with 100 ns MD simulation and the trajectory of each simulated system was saved every 100 ps.

Molecular mechanics generalized born surface area (AMIGBSA) calculation. For the saved trajectories of each MD simulation, MM/GBSA method was applied to calculate the binding energies of TRPM8 under the treatment of different ligands. In order to calculate the mean binding energy, 50 snapshots were extracted from each trajectory every 200 ps from 90 to 100 ns using the following formula:

ΔE_{bind}=ΔE_{MM}+ΔE_{SOL}=ΔE_{MM}+ΔE_{GB}+ΔE_{SA}

where ΔE_{bind} is the binding energy, and ΔE_{MM} denotes the sum of molecular mechanical energies in vacuo and can be further divided into the contributions from electrostatic, van der Waals, and internal energies. This term could be computed through a molecular mechanics method. ΔE_{SOL} is the solvation energy which includes the polar solvation energy (ΔE_{GB}) calculated with the Generalized Born (GB) approximation model and the non-polar part (ΔE_{SA}) obtained by fitting solvent accessible surface area (SASA) with the linear combinations of pairwise overlaps (LCPO) model. Additionally, the energies of each residue were decomposed into the backbone and side-chain atoms. Through energy decomposition, it was possible to analyze the contributions of the key residues to the biological activity.

Chemistry. Thin layer chromatography was performed on Analtech silica gel GF 250 micron TLC plates. The plates were visualized with a 254 nM UV light and staining with iodine. Flash chromatography was carried out on F60 silica gel, 230-400 mesh, 60 Å (Silicycle© SiliaFlash®). Automated flash chromatography was carried out on a Biotage Isolera Prime automated flash purification system with a multi-variable wavelength (200-400 nm) detector (Model ISO-PSV), using Biotage SNAP Ultra cartridges (10 g or 25 g) packed with Biotage HP-Sphere™ 25 micron spherical silica, using a binary gradient of solvent A (dichloromethane)/solvent B (diethyl ether) (for 2: 99/1→94/6, for Compound 4: 100/0→96:4, for Compounds 5, 6, 9, 10, 12, 14, 15: 100/0→98/2, for Compound 7: 96/4→94/6; for Compound 13: 100/0→94/6), at a flow rate of 12 or 25 mL/min. Eluted peaks were monitored at 254 and 280 nm. NMR was recorded on a Bruker Ascent 400 MHz NMR using CDCl₃. Mass spectra were obtained on an Agilent 1290 Infinity and 6490 Triple Quad LC/MS using electrospray ionization (ESI) mode. LC-UV-MS of Compound 2 and its isomers (2 Å, 2B, 2C), and Compounds 10 and 12 were carried out using an Accela UHPLC system (Thermo Scientific, San Jose, Calif., USA) consisting of an Accela 1250 pump, open autosampler, and PDA detector followed by connection to a Thermo Q Exactive Orbitrap mass spectrometer. Chromatographic separation was performed on an Agilent Eclipse XDB C18 column (1.8 μm particles and dimensions of 2.1 mm×100 mm, with a 2.1 mm×5 mm guard column) using mobile phases consisting of A: 0.1% formic acid in LC-MS grade water (v/v) and B: 0.1% formic acid in acetonitrile (v/v) and two different elution methods. A gradient elution method was used for Compound 2 and its isomers (2 Å, 2B, 2C), which consisted of 0% B to 95% B over 8 minutes followed by column cleaning and equilibration time for a total of 20 minutes. An isocratic elution method was used for Compounds 10 and 12, which was 58% B held constant for 20 minutes. For both chromatographic methods, the flow rate was 300 μl/min, and 5 μl of sample were injected. UV detection was achieved using the PDA detector over the range of 200 to 600 nm and specific channel wavelengths were monitored (280 nm, 254 nm, and 214 nm). The mass spectrometer parameters consisted of electrospray ionization in positive ion mode, scanning in full MS mode at 3.7 kV electrospray voltage, resolution setting of 70,000, AGC target 1e6, maximum IT 100 ms, and scan range m/z 150-1000. Data processing was performed using Qual Browser in Thermo Xcalibur version 4.1.31.9. Elemental analyses were performed by Atlantic Microlabs, Norcross, Ga. The purity of all final compounds was greater than 99.6%.

General amidation procedure. To a stirred solution of [1,1′-biphenyl]-4-carboxylic acid (1.00-1.5 equiv) in THE (0.1 M) was added EDCI (1.04-1.56 equiv), HOBt (1.04-1.56 equiv) and Et₃N (1.2-3.3 equiv), and the reaction was stirred at room temperature for 1 h. To this mixture was added a solution of the substituted amine (1.0-1.2 equiv) in THF, and the reaction was stirred at 40° C. for 2-3 hr or room temperature for 1-3 days. The reaction was diluted with EtOAc and H₂O. The combined organic layers were washed with satd. NaCl(aq), dried over Na₂SO₄, filtered and concentrated.

N-(2-isopropyl-5-methylcyclohexyl)-[1,1′-biphenyl]-4-carboxamide (Compound 2, a mixture of three isomers present as a 30:21:49 ratio by LCMS). Prepared according to the general procedure using 5-methyl-2-(propan-2-yl)cyclohexan-1-amine (62 mg, 0.40 mmol). The crude residue was purified via flash chromatography using hexane/diethyl ether/NH₄OH (100/0/0→95/5/0.5), followed by automated flash chromatography to afford 83 mg of the title material in 62% yield. ¹H NMR (mixture) (400 MHz, CDCl₃) δ 7.87-7.78 (m, 6H), 7.69-7.63 (m, 7H), 7.63-7.58 (m, 6H), 7.51-7.35 (m, 8H), 6.22 (d, J=9.0 Hz, 2H), 6.16 (d, J=8.4 Hz, 1H), 5.82 (d, J=9.5 Hz, 1H), 4.63-4.55 (m, 2H), 4.39 (tt, J=7.8, 3.9 Hz, 1H), 4.03 (qd, J=10.9, 3.9 Hz, 1H), 2.15-2.07 (m, 1H), 2.06-1.91 (m, 4H), 1.90-1.60 (m, 4H), 1.56-1.23 (m, 3H), 1.22-1.11 (m, 6H), 1.09-0.82 (m, 27H). MS (ESI) (mixture) m/z 336.30 (M+H)⁺. LCMS (mixture), R_{T} (Peak 1)=8.76 min, m/z (M+H)⁺=336.2; LCMS R_{T} (Peak 2)=8.84 min, m/z (M+H)⁺=336.2; LCMS R_{T} (Peak 3)=9.05 min, m/z (M+H)⁺=336.2. Anal. Calcd. For C23H₂₉NO (mixture): C, 82.34; H, 8.71; N, 4.18; found: C, 82.07; H, 8.58; N, 4.29.

Isomeric separation of Compound 2 (50 mg) was performed by preparative thin layer chromatography (TLC) (2000 micron, 20×20 cm, Analtech) using hexane: EtOAc: NH₄OH (79.6: 20.0:0.4). The plate was exposed twice to the aforementioned solvent system to afford isomer 2 Å (15 mg) and a mixture of isomers 2B and 2C (33 mg). Additional preparative TLC of the isomer 2B and 2C mixture was carried out using a gradient of hexane: EtOAc: NH₄OH (89.6: 9.9: 0.5→84.6/14.9/0.5) to afford isomer 2B (2.3 mg) and isomers 2B and 2C (16.6 mg).

Isomer 2 Å, isolated: ¹H NMR (400 MHz, CDCl₃) δ 7.86-7.84 (m, 2H), 7.70-7.67 (m, 2H), 7.65-7.62 (m, 2H), 7.51-7.48 (m, 2H), 7.44-7.39 (m, 1H), 6.24 (d, J=8 Hz, 1H), 4.64-4.59 (m, 1H), 2.08-2.03 (m, 1H), 1.99-1.95 (m, 1H), 1.85-1.81 (m, 1H), 1.54 (br s, 1H), 1.48-1.41 (m, 1H), 1.28 (s, 1H), 1.22-1.04 (m, 3H), 1.00-0.92 (m, 9H). LCMS R_{T}=9.26 min, m z (M+H)⁺=336.2. TLC (SiO₂) R_{f} 0.37 (8:2 hexane: diethyl ether+2 drops NH₄OH).

Isomer 2B, isolated: 1H NMR (400 MHz, CDCl₃) δ 7.85-7.82 (m, 2H), 7.67-7.60 (m, 4H), 7.49-7.36 (m, 3H), 5.79 (d, J=8 Hz, 1H), 4.03 (qd, J=10.9, 3.8 Hz, 1H), 2.16-2.12 (m, 1H), 2.05-1.85 (m, 1H), 1.79-1.70 (m, 1H), 1.25-1.15 (m, 5H), 1.04-1.00 (m, 1H), 0.95-0.85 (m, 9H). LCMS R_{T}=8.85 min, m/z (M+H)⁺=336.2. TLC (SiO₂) R_{f} 0.31 (8:2 hexane: diethyl ether+2 drops NH₄OH).

Isomer 2C (obtained as a 70:30 ratio of 2B:2C): 1H NMR (400 MHz, CDCl₃) δ 7.87-7.83 (m, 2H), 7.69-7.67 (m, 2H), 7.64-7.62 (m, 2H), 7.51-7.47 (m, 2H), 7.43-7.39 (m, 1H), 6.18 (2C, d, J=8 Hz, 0.5H), 5.84 (2B, J=12 Hz, 1H), 4.39 (2C, tt, J=7.8, 3.9 Hz, 1H), 4.05 (2B, qd, J=10.9, 3.8 Hz, 1H), 2.16-2.12 (m, 1H), 2.05-1.99 (m, 1H), 1.89 (br s, 1H), 1.81-1.61 (m, 4H), 1.57-1.54 (m, 1H), 1.48-1.15 (m, 4H), 1.06-0.98 (2C, m, 4H), 0.96-0.88 (2B, m, 9H). LCMS R_{T} (Isomer B)=8.93 min, m/z (M+H)⁺=336.2. LCMS R_{T} (Isomer C)=9.00 min, m z (M+H)⁺=336.2. TLC (Isomer C) (SiO₂) R_{f} 0.28 (8:2 hexane: diethyl ether+2 drops NH₄OH).

N-cyclohexyl-[1,1′-biphenyl]-4-carboxamide (Compound 3). Prepared according to the general procedure using cyclohexylamine-HCl (342 mg, 2.52 mmol). The crude residue was purified via trituration using 30 mL hexane/ether (90/10), followed by trituration with 15 mL MeOH to afford 536 mg of the title material in 76% yield. ¹H NMR (400 MHz, CDCl₃) δ 7.86-7.79 (m, 2H), 7.68-7.57 (m, 4H), 7.51-7.42 (m, 2H), 7.42-7.34 (m, 1H), 6.00 (d, J=8.1 Hz, 1H), 4.01 (dddd, J=14.7, 10.7, 8.0, 4.0 Hz, 1H), 2.07-2.04 (m, 2H), 1.77 (dt, J=13.7, 3.7 Hz, 2H), 1.67 (dt, J=12.7, 3.7 Hz, 1H), 1.52-1.37 (m, 2H), 1.33-1.14 (m, 3H). MS (ESI) m/z 280.3 (M+H)⁺. Anal. Calcd. For C₁₉H₂₁NO.0.3H₂O: C, 80.13; H, 7.65; N, 4.92; found: C, 80.15; H, 7.37; N, 4.99.

N-(bicyclo[2.2.1]heptan-2-yl)-[1,1′-biphenyl]-4-carboxamide (Compound 4, single isomer). Prepared according to the general procedure using bicyclo[2.2.1]heptan-2-amine HCl (106 mg, 0.72 mmol). The crude residue was purified via automated flash chromatography to afford 80 mg of the title material in 38% yield. ¹H NMR (400 MHz, CDCl₃) δ 7.88-7.77 (m, 2H), 7.69-7.58 (m, 4H), 7.51-7.42 (m, 2H), 7.47-7.34 (m, 1H), 6.17 (d, J=7.2 Hz, 1H), 4.35 (dddd, J=11.3, 6.8, 4.4, 1.6 Hz, 1H), 2.59 (br s, 1H), 2.39-2.14 (m, 2H), 1.71-1.44 (m, 2H), 1.39 (ddt, J=9.9, 3.2, 1.7 Hz, 1H), 1.34-1.23 (m, 1H), 0.86 (ddd, J=12.9, 4.7, 3.0 Hz, 1H). MS (ESI) m/z 292.4 (M+H)⁺. Anal. Calcd. For C₂₀H₂₁NO: C, 82.44; H, 7.26; N, 4.81; found: C, 82.05; H, 7.10; N, 4.84.

N-cycloheptyl-[1,1′-biphenyl]-4-carboxamide (Compound 5). Prepared according to the general procedure using cycloheptylamine (100 mg, 0.88 mmol). The crude residue was purified via automated flash chromatography to afford 200 mg of the title material in 77% yield. ¹H NMR (400 MHz, CDCl₃) δ 7.86-7.78 (m, 2H), 7.69-7.57 (m, 4H), 7.50-7.43 (m, 2H), 7.42-7.35 (m, 1H), 6.07 (d, J=8.1 Hz, 1H), 4.18 (ddt, J=12.8, 8.4, 3.6 Hz, 1H), 2.17-1.98 (m, 3H), 1.73-1.60 (m, 4H), 1.58-1.49 (m, 5H). MS (ESI) m/z 294.3 (M+H)⁺. Anal. Calcd. For C₂₀H₂₃NO: C, 81.87; H, 7.90; N, 4.77; found: C, 81.84; H, 7.76; N, 4.67.

N-(bicyclo[3.2.1]octan-3-yl)-[1,1′-biphenyl]-4-carboxamide (Compound 6). Prepared according to the general procedure using bicyclo[3.2.1]octan-3-amine-HCl (90 mg, 0.55 mmol). The crude residue was purified via automated flash chromatography to afford 54 mg of the title material in 32% yield. ¹H NMR (400 MHz, CDCl₃) δ 7.84-7.77 (m, 2H), 7.70-7.56 (m, 4H), 7.51-7.42 (m, 2H), 7.42-7.34 (m, 1H), 5.86 (d, J=8.4 Hz, 1H), 4.33 (tdt, J=11.5, 8.4, 5.8 Hz, 1H), 2.31 (br s, 2H), 2.06-1.97 (m, 2H), 1.69 (br s, 4H), 1.54-1.44 (m, 1H), 1.40 (d, J=11.2 Hz, 1H), 1.33-1.23 (m, 2H). MS (ESI) m/z 306.3 (M+H)⁺. Anal. Calcd. For C₂₁H₂₃NO: C, 82.58; H, 7.59; N, 4.59; found: C, 82.35; H, 7.68; N, 4.58.

N-cyclooctyl-[1,1′-biphenyl]-4-carboxamide (Compound 7). Prepared according to the general procedure using cyclooctylamine (500 mg, 3.93 mmol). The crude residue was purified via flash chromatography using hexane/EtOAc (95/5→30/70), followed by automated flash chromatography to afford 605 mg of the title material in 50% yield. ¹H NMR (400 MHz, CDCl₃) δ 7.86-7.78 (m, 2H), 7.68-7.56 (m, 4H), 7.51-7.42 (m, 2H), 7.42-7.34 (m, 1H), 6.08 (d, J=8.1 Hz, 1H), 4.23 (ddq, J=12.0, 8.0, 3.7 Hz, 1H), 2.01-1.89 (m, 2H), 1.78-1.60 (m, 12H). MS (ESI) m/z 308.3 (M+H)⁺. Anal. Calcd. For C₂₁H₂₅NO: C, 82.04; H, 8.20; N, 4.56; found: C, 81.93; H, 8.10; N, 4.58.

N-(decahydronaphthalen-1-yl)-[1,1′-biphenyl]-4-carboxamide (Compound 8, mixture of three isomers). Prepared according to the general procedure using decahydronaphthalen-1-amine HCl (91 mg, 0.483 mmol). The crude residue was purified via trituration using 15 mL MeOH to afford 136 mg of the title material in 84% yield. ¹H NMR (400 MHz, CDCl₃) δ 7.87-7.79 (m, 6H), 7.72-7.57 (m, 12H), 7.51-7.42 (m, 6H), 7.42-7.35 (m, 3H), 6.05 (d, J=8.5 Hz, 1H), 5.82 (d, J=9.3 Hz, 2H), 4.15 (ddt, J=11.8, 7.9, 4.0 Hz, 1H), 3.89-3.75 (m, 2H), 2.16-2.06 (m, 3H), 1.97-0.81 (m, 45H). MS (ESI) m/z 334.30 (M+H)⁺. Anal. Calcd. For C23H27NO-0.3 H₂O: C, 81.52; H, 8.21; N, 4.13; found: C, 81.44; H, 7.94; N, 4.16.

N-(1,2,3,4-tetrahydronaphthalen-1-yl)-[1,1′-biphenyl]-4-carboxamide (Compound 9). Prepared according to the general procedure using 1,2,3,4-tetrahydro-1-naphthylamine (250 mg, 1.70 mmol). The crude residue was purified via automated flash chromatography to afford 384 mg of the title material in 69% yield. ¹H NMR (400 MHz, CDCl₃) δ 7.90-7.82 (m, 2H), 7.69-7.57 (m, 4H), 7.51-7.43 (m, 2H), 7.42-7.34 (m, 2H), 7.24-7.12 (m, 3H), 6.37 (d, J=8.4 Hz, 1H), 5.48-5.38 (m, 1H), 2.94-2.75 (m, 2H), 2.24-2.12 (m, 1H), 2.03-1.86 (m, 3H). MS (ESI) m/z 328.20 (M+H)⁺. Anal. Calcd. For C23H21NO: C, 84.37; H, 6.46; N, 4.28; found: C, 84.14; H, 6.37; N, 4.17.

N-(decahydronaphthalen-2-yl)-[1,1′-biphenyl]-4-carboxamide (Compound 10, a mixture of two isomers present as a 86:13 ratio by LCMS) and N-((2SR,9RS,10SR)-decahydronaphthalen-2-yl)-[1,1′-biphenyl]-4-carboxamide (Compound 12, single isomer). Prepared according to the general procedure using decahydronaphthalen-2-amine (63 mg, 0.41 mmol). The crude residue was purified via automated flash chromatography to afford 10 (111 mg, 80%) and 12 (28 mg, 20%). ¹H NMR (mixture) (400 MHz, CDCl₃, 10) δ 7.87-7.78 (m, 3H), 7.69-7.57 (m, 6H), 7.50-7.42 (m, 3H), 7.42-7.35 (m, 1H), 6.00 (d, J=8.1 Hz, 1H), 5.94 (d, J=8.4 Hz, 0.31H), 4.20 (s, 0.26H), 4.00 (tdt, J=11.9, 8.2, 4.2 Hz, 1H), 2.18-1.59 (m, 9H), 1.57-0.84 (m, 8H). MS (ESI) (mixture) m/z 334.3 (M+H)⁺. LCMS (mixture) R_{T} (Peak 1 (major isomer) corresponds to 12)=7.91 min, m/z (M+H)⁺=334.2; LCMS R_{T} (Peak 2 (minor isomer))=8.70 min, m/z (M+H)⁺=334.2. Anal. Calcd. For C₂₃H₂₇NO (mixture): C, 82.84; H, 8.16; N, 4.20; found: C, 82.57; H, 7.97; N, 4.20. TLC (major isomer, corresponds to 12) (SiO₂) R_{f} 0.54 (8:2 hexane: EtOAc). TLC (minor isomer) (SiO₂) R_{f} 0.59 (8:2 hexane: EtOAc).

¹H NMR (400 MHz, CDCl₃, Compound 12, single isomer) δ 7.87-7.79 (m, 2H), 7.70-7.57 (m, 4H), 7.51-7.43 (m, 2H), 7.42-7.35 (m, 1H), 6.00 (d, J=8.1 Hz, 1H), 4.01 (tdt, J=12.0, 8.4, 4.3 Hz, 1H), 1.96-1.58 (m, 8H), 1.56-1.20 (m, 8H). MS (ESI) (single isomer) m/z 334.3 (M+H)⁺. LCMS (single isomer) R_{T}=8.04 min, m/z (M+H)⁺=334.2. Anal. Calcd. For C₂₃H₂₇NO (single isomer): C, 82.84; H, 8.16; N, 4.20; found: C, 82.73; H, 8.13; N, 4.14. TLC (SiO₂) R_{f} 0.54 (8:2 hexane: EtOAc).

¹H, ¹³C, gCOSY, NOESY, gHSQCAD, gHMBCAD, gH2BC of Compound 12. All experiments were run on an Agilent DDR2 500 MHz spectrometer equipped with an OneNMR probe running at 499.90 MHz and 125.71 MHz for 1H and ¹³C resonance frequencies, respectively. Experiments were run at ambient temperature using CDCl₃ as the solvent. ¹H spectra were referenced against residual protonated solvent (7.26 ppm) and ¹³C to 77 ppm. Spectra were processed using MestReNova (12.0.3) NMR processing program.

¹H NMR: 10 second relaxation delay, 8013 Hz spectral window, 2.04 second acquisition time, 8 scans. Baseline correction applied using Whittaker smoother (automatic detection).

¹³C NMR: Broadband ¹H-decoupled, 1 second relaxation delay, 512 scans, 31250 Hz spectral window, 1.05 second acquisition time. Baseline correction applied using Whittaker smoother (automatic detection) and 0.5 Hz exponential multiplication was applied.

gCOSY: 1 second relaxation delay, 4 scans per increment, 200 increments, 4882 Hz spectral window, 0.15 second acquisition time. F1 and F2 were multiplied by Sine Square II function (50%), as implemented by MestReNova, prior to Fourier transform. Linear prediction (Zhu-Bax) to 1024 points in F1 was applied. Baseline correction was applied to both dimensions using Whittaker smoother (automatic detection).

gH2BC: 1 second relaxation delay, 8 scans per increment, 200 increments, 8013, 25141 Hz spectral window (¹H, and ¹³C, respectively), 0.15 second acquisition time. F1 was multiplied by a Gaussian and F2 by Sine Square II function (50%), as implemented by MestReNova, prior to Fourier transform. Baseline correction was applied to both dimensions using Whittaker smoother (automatic detection). Linear prediction (Zhu-Bax) to 2048 points in F1 was applied.

gHMBCAD: 1 second relaxation delay, 8 scans per increment, 320 increments; 8013, 30166 Hz spectral window (¹H, and ¹³C, respectively), 0.15 second acquisition time. F2 was multiplied by a Gaussian and F1 by Sine Square II function (50%), as implemented by MestReNova, prior to Fourier transform. Baseline correction was applied to both dimensions using Whittaker smoother (automatic detection). Linear prediction (Zhu-Bax) to 2048 points in F1 was applied.

gHSQCAD: 1 second relaxation delay, 4 scans per increment, 200 increments; 8013, 25141 Hz spectral window (¹H, and ¹³C, respectively), 0.15 second acquisition time. F1 and F2 were multiplied by a Gaussian prior to Fourier transform. Baseline correction was applied to both dimensions using Whittaker smoother (automatic detection). Linear prediction (Zhu-Bax) to 2048 points in F1 was applied.

N-(1,2,3,4-tetrahydronaphthalen-2-yl)-[1,1′-biphenyl]-4-carboxamide (Compound 11). Prepared according to the general procedure using 1,2,3,4-tetrahydronaphthalen-2-amine-HCl (197 mg, 1.34 mmol). The crude residue was purified via trituration with 15 mL MeOH to afford 328 mg of the title material in 75% yield. ¹H NMR (400 MHz, CDCl₃) δ 7.95-7.93 (m, 2H), 7.77-7.71 (m, 4H), 7.57-7.50 (m, 3H), 7.37 (s, 1H), 7.23-7.26 (m, 3H), 6.29 (d, J=4 Hz, 1H), 4.65 (br s, 1H), 3.39 (d, J=20 Hz, 1H), 3.07 (br s, 1H), 2.88-2.94 (m, 1H), 2.33-2.30 (m, 1H), 2.09-2.00 (m, 1H), 1.70 (s, 1H). MS (ESI) m/z 328.2 (M+H)⁺. Anal. Calcd. For C₂₃H₂₁NO: C, 84.37; H, 6.46; N, 4.28; found: C, 84.10; H, 6.42; N, 4.25.

N-(spiro[5.5]undecan-3-yl)-[1,1′-biphenyl]-4-carboxamide (Compound 13). Prepared according to the general procedure using spiro[5.5]undecan-3-amine-HCl (90 mg, 0.44 mmol). The crude residue was purified via automated flash chromatography to afford 130 mg of the title material in 84% yield. ¹H NMR (400 MHz, CDCl₃) δ 7.86-7.79 (m, 2H), 7.69-7.57 (m, 4H), 7.51-7.43 (m, 2H), 7.42-7.35 (m, 1H), 6.02 (d, J=8.1 Hz, 1H), 3.97 (dddd, J=14.9, 10.7, 8.3, 4.2 Hz, 1H), 1.88 (ddd, J=13.1, 6.2, 3.8 Hz, 3H), 1.67 (d, J=13.6 Hz, 3H), 1.42 (br s, 8H), 1.25 (td, J=12.7, 4.4 Hz, 4H). MS (ESI) m/z 348.4 (M+H)⁺. Anal. Calcd. For C₂₄H₂₉NO: C, 82.95; H, 8.41; N, 4.03; found: C, 82.99; H, 8.42; N, 3.99.

N-(spiro[4.5]decan-8-yl)-[1,1′-biphenyl]-4-carboxamide (Compound 14). Prepared according to the general procedure using spiro[4.5]decan-8-amine-HCl (91 mg, 0.48 mmol). The crude residue was purified via automated flash chromatography to afford 110 mg of the title material in 68% yield. ¹H NMR (400 MHz, CDCl₃) δ 7.87-7.79 (m, 2H), 7.69-7.57 (m, 4H), 7.51-7.43 (m, 2H), 7.42-7.35 (m, 1H), 6.01 (d, J=8.1 Hz, 1H), 4.05-3.93 (m, 1H), 2.00-1.91 (m, 2H), 1.67-1.60 (m, 3H), 1.58-1.31 (m, 11H). MS (ESI) m/z 334.3 (M+H)⁺. Anal. Calcd. For C₂₃H₂₇NO: C, 82.84; H, 8.16; N, 4.20; found: C, 82.57; H, 8.16; N, 4.22.

N-(adamantan-1-yl)-[1,1′-biphenyl]-4-carboxamide (Compound 15). Prepared according to the general procedure using 1-adamantanamine-HCl (100 mg, 0.54 mmol). The crude residue was purified via automated flash chromatography to afford 110 mg of the title material in 62% yield. ¹H NMR (400 MHz, CDCl₃) δ 7.83-7.75 (m, 2H), 7.67-7.57 (m, 4H), 7.51-7.42 (m, 2H), 7.42-7.34 (m, 1H), 5.84 (s, 1H), 2.15 (br s, 9H), 1.73 (br s, 6H). MS (ESI) m/z 332.3 (M+H)⁺. Anal. Calcd. For C₂₃H₂₅NO.0.1H₂O: C, 82.89; H, 7.62; N, 4.20; found: C, 82.87; H, 7.47; N, 4.18.

In Vitro Pharmacology

Evaluation of TRPM8 agonist and antagonist activity using Ca²⁺ imaging. Fura-2 based Ca²⁺-imaging was used to evaluate the effects of the synthesized compounds on icilin-evoked Ca²⁺ signals. HEK-293 cells stably expressing human TRPM8 (a kind gift from Professor Thomas Voets, Laboratory of Ion Channels Research, KU Leuven, Belgium) were first loaded with Fura-2 AM for 45 min and then another 40 min was allowed for de-esterification of the dye. The cells were then bathed in Ca²⁺-free HBSS buffer and incubated with a test compound for 5 min, and then icilin (500 nM, from Tocris)—the well-known TRPM8 agonist, was added to the bath solution. 1 min after icilin addition, 2 mM Ca²⁺ was added to the bath solution.

Ratio fluorescence images were captured using a QIClick™ digital CCD camera (QImaging, BC, Canada) mounted on a Nikon Eclipse Ti-S Microscope. Consecutive excitation was provided by a Dual OptoLED Power Supply (Cairn), alternating between both 355 nm (F355) and 380 nm (F380) wavelength LEDs. Emission fluorescence was collected at 510 nm (470 nm-550 nm). 12-bit images were acquired in every 5 seconds with MetaFluor® (Molecular Devices, USA). The fluorescence at each time-point was extracted for both 355 nm and 380 nm wavelengths, corrected for autofluorescence and the 355 nm/380 nm ratios (F355/F380) were then calculated to represent Ca²⁺ influx. All Ca²⁺ imaging experiments were done in room (22° C.) temperature. For comparison, peak Ca²⁺ response for each compound was normalized to that of icilin (500 nM). RQ-00203078 was purchased from Tocris.

For obtaining the concentration-response profile of icilin, Fura-2 loaded HEK-293 cells stably expressing human TRPM8 in different petri dishes were bathed in Ca²⁺ free HBSS and were first incubated with icilin in graded concentrations (10⁻⁹ to 10⁻⁶M). 1 min after icilin addition, 2 mM Ca²⁺ was added to the bath solution and the resultant Ca²⁺ influx (as Fura-2 fluorescence ratios) were recorded. EC₅₀ was obtained from the corresponding concentration-response curve.

To determine the antagonist activity of the designed compounds (e.g., from Table 1 below), cells bathed in Ca²⁺-free Hank's balanced salt solution (Ca²⁺ free HBSS) were first incubated for 3 min with different concentrations (1 nM, 3 nM, 10 nM, 30 nM, 100 nM, 300 nM, 1 μM, 3 μM, 10 μM, 30 μM and 100 μM) of Compounds 2-15. Then 500 nM icilin was added and after another 1 min, Ca²⁺-free HBSS was replaced by HBSS containing 2 mM free Ca²⁺ and the resultant Ca²⁺ signal was measured. The TRPM8 antagonist RQ-00203078 was used as a positive control and for comparison purposes due to its reported high nanomolar potency (<10 nM) at hTRPM8 vs. other antagonists in the literature. Compounds with IC₅₀ values ≤100 nM were tested alone at a single concentration of 10 μM for their intrinsic (agonist) ability to induce Ca²⁺ influx, and compared to the effect of 500 nM icilin.

TRPA1 and TRPV1 selectivity assays. The Ca²⁺ flux assay utilized Fluo-8 Ca²⁺ dye and fluorescence were measured at excitation wavelength of 470-495 nm and emission wavelength of 515-575 nm using FLIPR^{TETRA} μlate reader (Molecular Devices). A total of eight compounds were tested for both agonist (intrinsic) and antagonist activity at concentrations corresponding to approximately IC₉₀ against TRPM8 and 10× IC₉₀ to demonstrate a 10-fold selectivity. Recombinant HEK293 cell lines stably expressing human TRPA1 (Eurofins Cat #CYL3066) and human TRPV1 (Eurofins Cat #CYL3063) were used for the study. Cells were cultured and maintained in DMEM-F12 medium supplemented with 10% FBS, 1% Non-Essential amino acids, and 400 μg/ml Geneticin. Allyl isothiocyanate (AITC, Millipore Sigma) and capsaicin (Tocris) were used as reference agonists, whereas ruthenium red (Tocris) and capsazepine (Millipore Sigma) were reference antagonists against TRPA1 and TRPV1, respectively. For the experiments, cells were plated in flat-bottom collagen-coated 384 well plates and incubated at 37° C. 5% CO₂. After 24 h of incubation, the media was aspirated and 40 μl of dye loading buffer (modified Hanks Balanced Salt Solution (HBSS) where HBSS was supplemented to contain 20 mM HEPES and 2.5 mM Probenecid at pH 7.4) containing Fluo-8 Ca²⁺ dye at 5 μg/ml was added to the cells in each well and incubated at 30° C. (5% CO₂) in a humidified chamber for 80 minutes prior to starting the FLIPR Tetra protocol. Test compounds were initially dissolved in DMSO to make 300× the final assay concentrations, and then diluted in assay buffer (modified Hanks Balanced Salt Solution (HBSS) where HBSS was supplemented to contain 20 mM HEPES at pH 7.4) to the final dilutions. For the agonist assay, test compound(s), vehicle controls, and reference agonist (at Eₘₐₓ=300 μM (AITC) and 3 μM (capsaicin)) were added to the assay plate for 180 seconds after a fluorescence baseline was established. For the antagonist assay, cells were pre-incubated with the test compounds or positive controls (known antagonists) for five (5) minutes at room temperature and then challenged with EC₈₀ concentration of reference agonists (AITC EC₈₀=10 μM, capsaicin EC₈₀=0.1 μM) for 180 seconds after establishment of a fluorescence baseline. Agonist: 25% activation when normalized to Eₘₐₓ. Antagonist: 25% inhibition when normalized to EC₈₀.

Whole Cell Patch Clamp Electrophysiology

HEK-293 Cell culture. Human embryonic kidney (HEK) 293 cells (ATCC CRL-1573) were cultured in growth medium comprising 90% Dulbecco's modified Eagle's medium, 10% fetal bovine serum, 100 U·mL-1 penicillin-streptomycin, and 2 mM L-glutamine (Gibco). Cells were cultured in 35 mm polystyrene dishes (Falcon) at 37° C. in the presence of 5% CO₂. Cells were transiently transfected with human TRPM8 in a pIRES2 plasmid containing EGFP as a reporter. This plasmid expresses bicistronic mRNA with an internal ribosome entry site (IRES) positioned between the TRPM8 gene and the fluorescent protein reporter gene such that the reporter is not covalently fused to TRPM8. Transient transfection was performed using Fugene 6 transfection reagent (Promega) and 0.5 μg of plasmid in a 35 mm dish (Falcon) at a ratio of 3 μL transfection reagent per μg of plasmid according to manufacturer protocol. HEK-293 cells were authenticated by polymorphic genetic marker testing (DNA Diagnostics Center Medical).

Whole-cell patch-clamp electrophysiology. 48 hours following transfection, cells were released from culture dishes by brief exposure to 0.25% trypsin/EDTA (Gibco), resuspended in supplemented DulDMEM, plated on glass coverslips and allowed to recover for 1-2 h at 37° C. in 5% CO₂. Cells that exhibited green fluorescence, indicating successful transfection, were selected for electrophysiology measurements. Cells were placed in a chamber with extracellular solution containing (in mM) NaCl 132, KCl 4.8, MgCl₂ 1.2, CaCl₂ 2, HEPES 10, and glucose 5, with the pH adjusted to 7.4 using NaOH and the osmolality adjusted to 310 mOsm using sucrose. Pipettes were filled with a solution containing (in mM) K+ gluconate 135, KCl 5, MgCl₂ 1, EGTA 5, and HEPES 10; pH was adjusted to 7.2 with KOH, and osmolality was adjusted to 300 mOsm using sucrose. Chemicals were obtained from Sigma-Aldrich. Osmolality was measured using a Vapro 5600 vapor pressure osmometer (Wescor). Whole-cell voltage-clamp current measurements were performed using an Axopatch 200B amplifier (Axon Instruments) and pClamp 10.3 software (Axon Instruments). Data was acquired at 2 kHz and filtered at 1 kHz. Patch pipettes were pulled using a P-2000 laser puller (Sutter Instruments) from borosilicate glass capillaries (World Precision Instruments) and heat-polished using a MF-830 microforge (Narishige). Pipettes had resistances of 2-5 MO as measured in the extracellular solution. A reference electrode was placed in a 2% agar bridge made with a composition similar to the extracellular solution, without glucose added. Experiments were performed at 23±1° C. Menthol and Compound 14 stock solutions were prepared in DMSO at 1 M and 3.6 mM, respectively. A series of perfusion solutions were prepared by diluting menthol to 500 μM menthol and Compound 14 to varying concentrations (from 1 to 1000 nM) in extracellular solution. A perfusion capillary was placed close to the cell and solution was perfused throughout the recording. Current response from each cell was measured under each concentration of Compound 14. Only one cell was measured per cover slip to prevent confounding effects of previous exposure to menthol and Compound 14.

Data analysis. For each cell, current magnitude at each concentration of Compound 14 was normalized by dividing by the maximum current measured at 500 μM menthol and without the antagonist. Normalized currents from six cells were averaged at each concentration of antagonist, and the standard error of the mean was calculated at each point. The data were plotted in SigmaPlot and fit to a single site competition model using the

$$equation = {I}_{min} + \frac{{I}_{max} - {I}_{min}}{1 + 1 {0}^{[ a n t a g o n i s t ] - log {IC}_{50}}} .$$

Where I is the current intensity and [antagonist] is the concentration of Compound 14 at a particular I value.

Hepatic Metabolic Stability

Stability in liver microsomes. Male CD-1 mouse liver microsomes (Lot#1710069) were purchased from XenoTech. The reaction mixture, minus NADPH, was prepared as described below. Compound 14 was added into the reaction mixture at a final concentration of 1 μM. The control compound, testosterone, was run simultaneously with Compound 14 in a separate reaction. An aliquot of the reaction mixture (without cofactor) was equilibrated in a shaking water bath at 37° C. for 3 minutes. The reaction was initiated by the addition of the cofactor, and the mixture was incubated in a shaking water bath at 37° C. Aliquots (100 μL) were withdrawn at 0, 10, 20, 30, and 60 minutes. Compound 14 and testosterone samples were immediately combined with 400 μL of ice-cold 50/50 acetonitrile (ACN)/H₂O containing 0.1% formic acid and internal standard to terminate the reaction. The samples were then mixed and centrifuged to precipitate proteins. All samples were assayed by LC-MS/MS using electrospray ionization. Analytical conditions are compared to the PARR at time 0 to determine the percent remaining at each time point. Half-lives and intrinsic clearance were calculated using GraphPad software, fitting to a single-phase exponential decay equation.

### Reaction Composition.

| | |
|---|---|
| Liver Microsomes | 0.5 mg/mL |
| NADPH (cofactor) | 1 mM |
| Potassium phosphate, pH 7.4 | 100 mM |
| Magnesium chloride | 5 mM |
| Analog 14 | 1 μM |

Testosterone control.

| | | CLᵢₙₜ | Acceptable |
|---|---|---|---|
| Control | Half-life | (mL/min/mg | range |
| compound | (min) | protein) | (t_{1/2}, min) |
| Testosterone | 3.7 | 0.372 | ≤15 |

Analytical method.

### Liquid Chromatography

Column: Thermo BDS Hypersil C18 30×2.1 mm, 3 μm, with guard column

M.P. Buffer: 25 mM ammonium formate buffer, pH 3.5

Aqueous Reservoir (A): 90% water, 10% buffer

Organic Reservoir (B): 90% acetonitrile, 10% buffer

Flow Rate: 350 μL/min

Gradient Program:

| Time | % | % |
|---|---|---|
| (min) | A | B |
| 0.0 | 100 | 0 |
| 1.0 | 0 | 100 |
| 1.5 | 0 | 100 |
| 1.6 | 100 | 0 |
| 2.5 | 100 | 0 |

Total Run Time: 2.5 minutes

Autosampler: 10 μL injection volume

Autosampler Wash: water/methanol/2-propanol: 1/1/1; with 0.2% formic acid

### Mass Spectrometer

Instrument: PE SCIEX API 4000

Interface: Turbo Ionspray

Mode: Multiple reaction monitoring

Method: 2.5 minute duration

Settings:

| Test article | ± | Q1 | Q3 | DP | EP | CE | CXP | IS |
|---|---|---|---|---|---|---|---|---|
| Analog 14 | + | 334.3 | 198.1 | 83 | 10 | 30 | 11 | 5500 |

TEM: 500; CAD: 7; CUR: 20; GS1: 20; GS2: 30

In Vivo Assays. C57-mice (≈30g) (Harlam, Holland) were used for the study. All experiments were approved by the Institutional Animal and Ethical Committee of the Universidad Miguel Hernandez where experiments were conducted and they were in accordance with the guidelines of the Economic European Community and the Committee for Research and Ethical Issues of the International Association for the Study of Pain. All parts of the study concerning animal care were performed under the control of veterinarians.

Wet-dog shakes assay. Icilin, a TRPM8 agonist, was dissolved in 20% DMSO and 1% Tween 80 in distilled water and injected intraperitoneally (i.p.) in a volume of 10 mg/kg. Each animal was acclimatized for 30 min for two consecutive days before icilin administration. The Compound 14 stock was prepared in DMSO and diluted in saline for injections. Gabapentin was dissolved in saline and administered s.c. at the dose of 25 mg/kg 60 min prior to icilin injection. Control animals received the vehicle injection.

Cold allodynia. Oxaliplatin (Tocris) was dissolved in water with gentle warming and was subcutaneously (s.c.) injected on days 1, 3 and 5 at a 6 mg/kg dose. The day 7 after administration, experiments were performed. Together with Oxaliplatin injection, saline and a 5% Mannitol solution were intraperitoneally injected to prevent kidney damage and dehydration. The Compound 14 stock was prepared in DMSO (Sigma-Aldrich) and diluted in saline for injections. Compounds at different doses (0.1 to 1 μg) was injected into the plantar surface (25 μL) of the right hind paw of mice. Cold chemical thermal sensitivity was assessed using acetone drop method. Mice were placed in a metal mesh cage and allowed to habituate for approximately 30 minutes in order to acclimatize them. Freshly dispensed acetone drop (10 μL) was applied gently on to the mid plantar surface of the hind paw. Cold chemical sensitive reaction with respect to paw licking was recorded as a positive response (nociceptive pain response). The responses were measured for 20-s with a digital stopwatch. For each measurement, the paw was sampled twice and the mean was calculated. The interval between each application of acetone was approximately 5 minutes.

Pharmacologic Evaluations

Evaluation of TRPM8 agonist and antagonist activity using Ca²⁺ imaging. Compounds 2-15 were evaluated for their ability to modulate TRPM8 functions with Fura-2 based Ca²⁺ imaging in HEK-293 cells stably expressing hTRPM8. The latter were a kind gift from Thomas Voets (Laboratory of Ion Channels Research, KU Leuven, Belgium). The concentration of intracellular free Ca²⁺ ([Ca²⁺]ᵢ) was monitored as a Fura-2 fluorescence ratio (F₃₅₅/F₃₈₀).

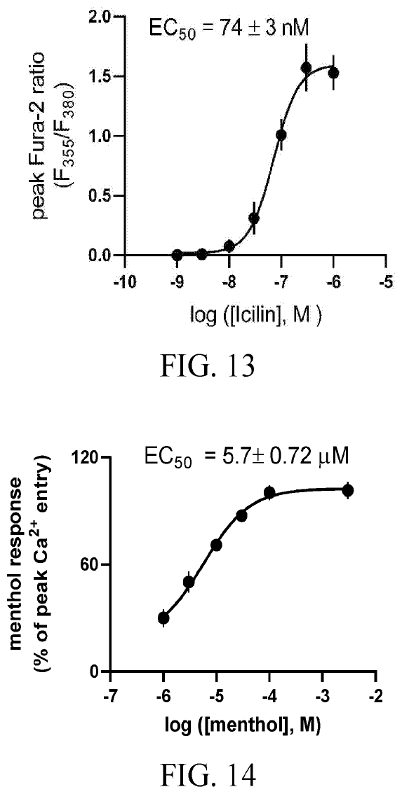

The compounds were assessed against Ca²⁺ influx ([Ca²⁺]ᵢ) evoked by icilin (AG-3-5). The latter was used at 500 nM, as maximum Ca²⁺ entry was found to occur at this concentration in our dose-response study. Icilin was chose to stimulate TRPM8 in the assay since it was markedly more potent (EC₅₀: ˜70 nM vs 6 μM) than (−)-menthol in the initial concentration-response studies (FIGS. 13-14) and this broadly agreed with previous reports.

TRPV1 and TRPA1 agonist and antagonist Ca²⁺ flux assay (selectivity profiling). Compounds with hTRPM8 IC₅₀ values ≤100 nM were evaluated for off-target effects at two related temperature-sensitive subtypes hTRPA1 and hTRPV1, using high-throughput FLIPR-based Ca²⁺ flux assays at Eurofins Pharma Discovery Services (St. Charles, Mo.). Compounds were evaluated at single concentrations corresponding to approximately 90% target coverage at TRPM8, followed by single point assays at 10-fold higher concentrations to demonstrate >10-fold selectivity. Agonist (intrinsic) activity was evaluated in recombinant HEK293 cell lines stably expressing either human TRPA1 or TRPV1, and normalized to the maximum effect (Eₘₐₓ) of the agonists allyl isothiocyanate or capsaicin, respectively. Antagonist activity was evaluated by measuring the ability of the analogs to inhibit AITC- or capsaicin-induced increases in cytosolic [Ca²⁺] by inhibiting Ca²⁺ influx in these cell lines, and compared to the effect of the ruthenium red (TRPA1) and capsazepine (TRPV1) used as reference antagonists.

Whole-cell patch clamp electrophysiology of Compound 14. The functional activity of Compound 14 in calcium flux assays was confirmed with whole-cell patch-clamp electrophysiology. Human TRPM8 whole-cell patch-clamp electrophysiology was done at 23±1° C. on human embryonic kidney (HEK) 293 cells (ATCC CRL-1573) authenticated by polymorphic genetic marker testing (DNA Diagnostics Center Medical) as described previously. A series of 500 μM menthol and variable concentration (from 0 to 1 μM) Compound 14 solutions were used. Cellular current response was measured at each concentration of Compound 14. To determine the IC₅₀, normalized currents from six cells were averaged at each concentration of antagonist, and the standard error of the mean was calculated at each point. The data were fit to a single site competition model to obtain IC₅₀. The IC₅₀ whole-cell patch-clamp experiments were deliberately performed against saturating (500 μM) menthol concentrations, because menthol currents are not Ca²⁺ dependent and give reproducible and robust currents. Similarly, studying the inhibitory effects of Compound 14 against saturating menthol, allows direct comparison to other published TRPM8 antagonism studies that used these conditions in whole-cell patch-clamp measurements.

In vitro metabolic stability. Compound 14 was evaluated for metabolic stability in mouse liver microsomes by Absorption Systems (Exton, Pa.), by determining the percent remaining at 0, 10, 20, 30 and 60 minutes when incubated with mouse liver microsomes in the presence of NADPH. The half-life (t_{1/2}) and in vitro intrinsic clearance (CLᵢₙₜ) of Compound 14 was calculated, in addition to the control (testosterone).

Results And Discussion

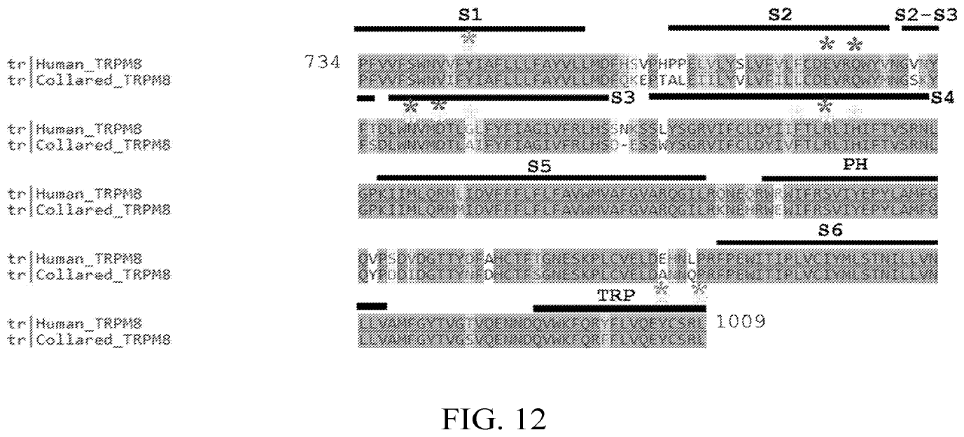

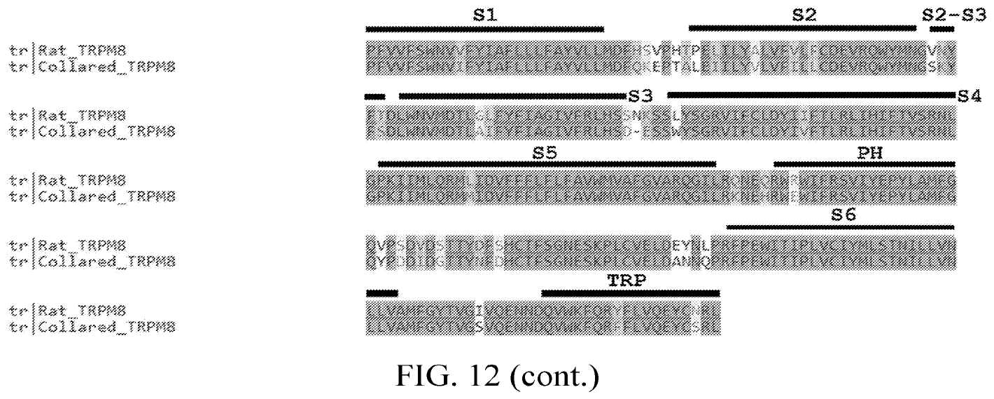

The available functional data on known TRPM8 antagonists including (−)-menthyl Compound 1 and AMG2850 are from rat and human TRPM8, for which there are yet to be solved structures, while the structure of the avian ortholog has recently been elucidated using cryo-EM. To first evaluate the degree of sequence conservation across these species, sequence alignment of the transmembrane S1-S4 helices (VSLD), S5-S6 helices (pore domain), pore helix (PH) and TRP helix of avian, human and rat TRPM8 was carried out (FIG. 12). As detailed above, residues within the VSLD and TRP helix are reported to be important for agonist (menthol) binding, agonist (menthol, icilin) response or efficacy and voltage sensitivity. Interestingly, the high degree of homology between full-length avian and human TRPM8 (83%) was retained within this transmembrane region containing the ligand binding site (86%); additionally there is a comparatively high sequence identity (87%) between the avian and rat orthologs in this same region (FIG. 12). The avian TRPM8 structure was therefore considered to be a reliable template for building a homology model of hTRPM8 with a view to first predicting plausible binding modes of known TRPM8 antagonists (−)-menthyl Compound 1 and AMG2850 and establishing a relevant structure-function correlation. The latter in turn, could inform the structure-based strategy for designing novel TRPM8 antagonist probes.

### Example 1—MD Simulations

No structure of hTRPM8 was available. The homology model of hTRPM8 was thus first built the based on the cryo-EM structure of the collared flycatcher Ficedula albicollis (TRPM8FA, PDB: 6BPQ), with which hTRPM8 shares 83% sequence identity to full length TRPM8FA and 86% in region containing the ligand binding site. Efforts were mainly focused on the transmembrane region of TRPM8, comprised of the VSLD (S1-S4 helices, also termed the sensing domain, SD) and pore domains, as well as the TRP helix, and the homology model of hTRPM8 was built with a sequence from Gln671 to Asn1010 (340 residues). The VSLD and TRP helix binds small molecule agonists, as discussed above, while the isolated VSLD (including the pre-S1 domain) is reported to recapitulate binding phenotypes that agree with functional activation of full-length hTRPM8 (1104 residues).

A Ca²⁺ ion was placed in the VLSD, coordinating to Glu782, Gln785, Asn799 and Asp802, to allow the human model to more closely resemble the cryo-EM structures of related human melastatin subtypes M2 (PDB 6MJ2) and M4 (PDB 6BQV), and provide structural context for our Ca²⁺ flux studies using the TRPM8 agonist icilin as an activator (Table 1). These structures capture a bound Ca²⁺ ion coordinating to four side chains oriented similarly in the VLSD, three of which are conserved among TRPM8, TRPM2 and TRPM4: Glu782 (TRPM8, S2 helix)/Glu843 (TRPM2, S2)/Glu828 (TRPM4, S2); Gln785 (TRPM8, S2)/Gln846 (TRPM2, S2)/Gln831 (TRPM4, S2); and Asn799 (TRPM8, S3)/Asn869 (TRPM2, S3)/Asn865 (TRPM4, S3). Taking into consideration the remaining non-conserved Ca²⁺ binding residue, Asp802 (S3) was chosen as the final chelating amino acid to satisfy the tetrahedral complex, corresponding to Asp868 in the M4 subtype (S3 helix). Notably, both Asn799 and Asp802 are well-known icilin sensitive residues. The recently reported icilin-bound structure of TRPM8 (PDB 6NR3, 3.4 Å) reveals a Ca²⁺ ion bound to these four residues within the now well-defined Ca²⁺ binding site, in agreement with the present homology model.

Sequentially, (−)-menthyl Compound 1, AMG2850 and Compound 14 were docked into the ligand binding site of hTRPM8, carried out 100 ns molecular dynamics (MD) simulations for each compound in hTRPM8, and analyzed the results as detailed below. (−)-menthyl 1, described as a potent and selective TRPM8 antagonist at rat TRPM8 in Ca²⁺ flux assays (IC₅₀ vs. 20 μM menthol, 20±2 nM; IC₅₀ vs. 0.25 μM icilin, 50±10 nM), is currently used in the literature as a TRPM8 tool molecule (also described as OMDM233), as well as AMG2850.

### Example 2—(−)-menthyl Compound 1 and hTRPM8

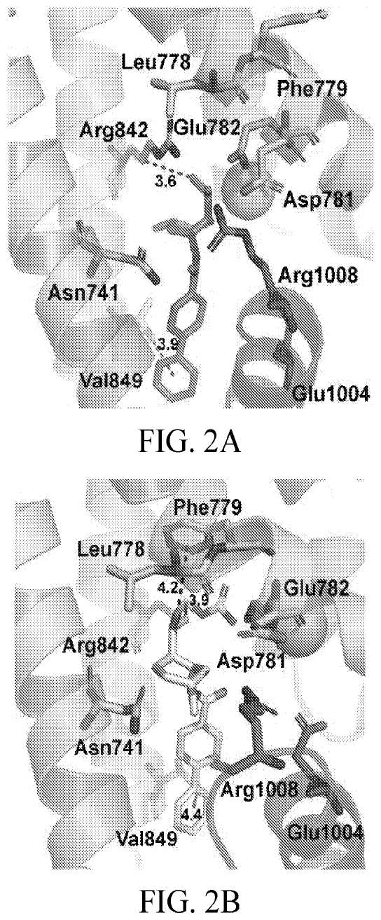

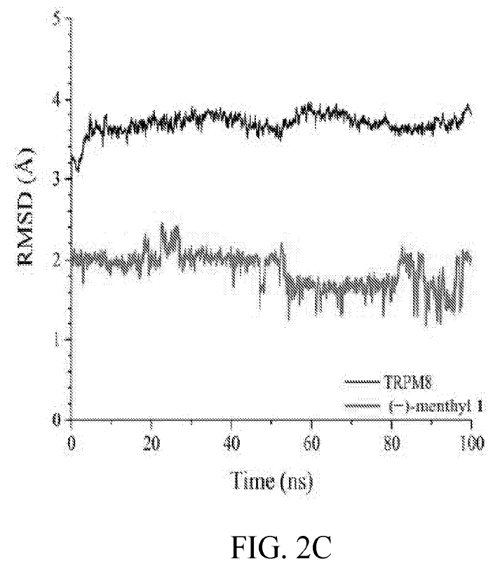

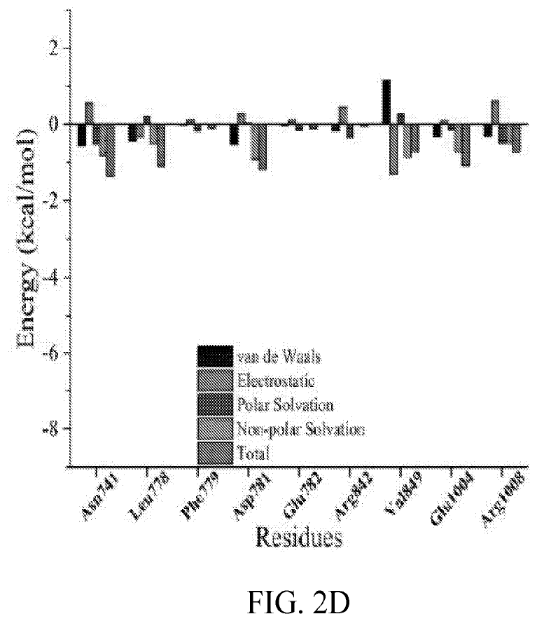

As shown in FIG. 2C, the results showed that both the root mean square deviation (RMSD) of hTRPM8 (about 3.9 Å) and (−)-menthyl Compound 1 (1.8 Å) kept stable after 50 ns during the simulation, indicating that the time scale of 100 ns is reasonable.

The biphenyl of Compound 1 kept stable during the MD simulation, projecting into the base of the binding cavity in an extended conformation, towards the TRP helix, which forms strong hydrophobic interactions with Asn741 (S1, ˜4.0 Å, not shown) and a conserved Val849 (S4, 4.4 Å), as shown in FIG. 2B, occupying a similar space as the corresponding methoxyphenyl of WS-12. This pose was consistent with SAR studies on this scaffold, in which a biphenyl substitution affords the highest TRPM8 potency among all analogs studied.

In contrast to the biphenyl moiety, it was found that the flexible part of Compound 1, the (−)-menthyl moiety, endures a change in conformation during the MD simulation, when comparing its binding mode between pre-MD (FIG. 2A) and post-MD (FIG. 2B). However, interactions between Compound 1 and some residues can be observed during the MD. For example, the (−)-menthyl of Compound 1 interacts with a conserved Arg1008 (TRP helix, 3.5 Å, not shown) (FIG. 2B), which assumes a folded conformation similar to that seen in the WS-12 bound structure, and may represent a common TRPM8 binding residue for small molecule ligands. To explore the contribution of these residues to the binding of Compound 1, the free energy was decomposed using the MM/GBSA method, as shown in FIG. 2D. From the energy decomposition, it was found that Arg1008 contributed greatly to the binding of Compound 1. Although the distance between Compound 1 and Arg842 (S4) was about 3.9 Å, the (−)-menthyl moiety approaches a conserved Leu778 (S2, ˜3.8 Å, not shown) during the MD simulation (FIG. 2B), overlapping with the (−)-menthyl and nitrophenyl binding regions of WS-12 and icilin, respectively, making Arg842 contribute less to the binding free energy than Leu778. The results are consistent with that of the free energy decomposition using the MM/GBSA method (FIG. 2D). Interestingly, the (−)-menthyl group does not contact Tyr745 (S1), as suggested for (−)-menthol by radioligand displacement studies, as well as the WS-12-bound structure, likely due to the anchoring nature of the biphenyl, which forms extensive hydrophobic interactions with the base of the orthosteric site. The studies here provide the first forays into investigating menthol-based antagonist mediated molecular events using the TRPM8 structural biology described to date.

### Example 3—AMG2850 and hTRPM8

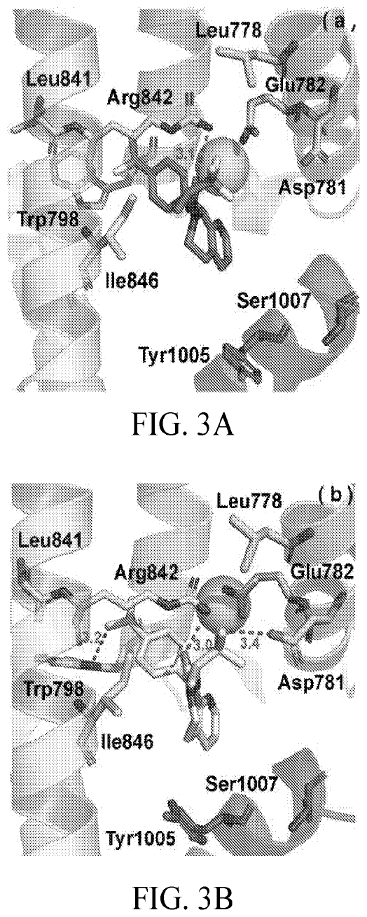

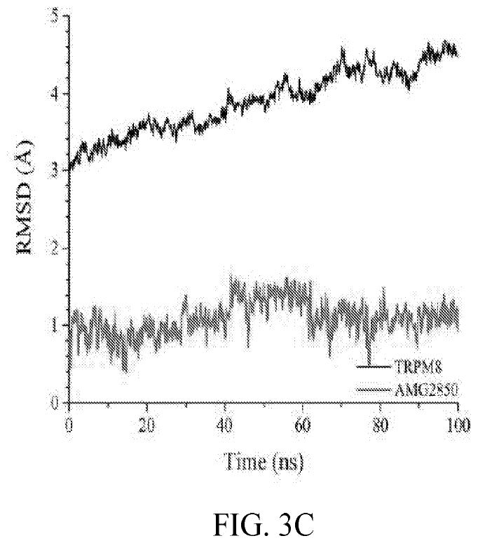

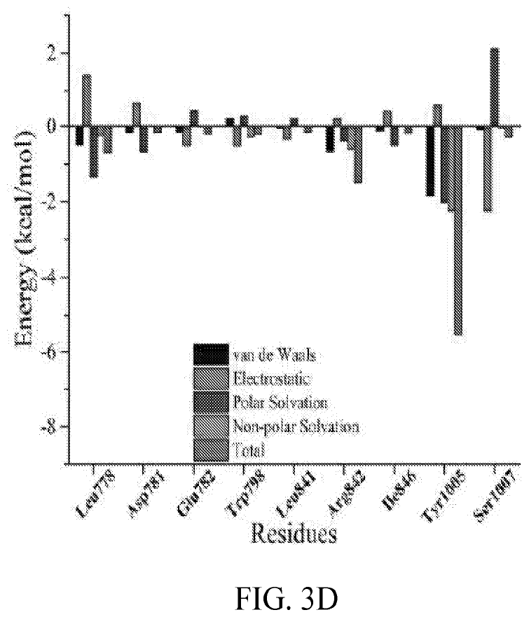

100 ns MD simulation was also performed for AMG2850 with hTRPM8. It was found that the RMSD of hTRPM8 equilibrates (about 4.4 Å) after 60 ns, as shown in FIG. 3C. The RMSD of AMG2850 kept stable at 1.6 Å (FIG. 3C). Due to the stability of AMG2850, the time scale of 100 ns was reasonable to explore its potential binding mode. Comparing the binding mode of AMG2850 between pre-MD (FIG. 3A) and post-MD (FIG. 3B), it was found that several important interactions between AMG2850 and hTRPM8 kept stable. For example, a conserved Arg842 (S4) forms a strong hydrogen bond with the urea carbonyl (3.1/3.0 Å) of AMG2850, similar to the WS-12 amide carbonyl and icilin dihydropyrimidinone carbonyl, suggesting a common H-bond acceptor feature for TRPM8 ligand recognition via this common residue. Consistent with this predicted interaction, SAR studies of the AMG2850 precursor series, containing a tetrahydrothienopyridine core, highlight the importance of the urea moiety, where deletion of this group negates TRPM8 activity (derivatives 24-26 reported by Tamayo et al.). The MD results also showed that a conserved Trp798 (S3) forms a hydrogen bond with the trifluorophenyl CF₃ group of AMG2850, with a distance of 3.2 Å, in agreement with SAR studies of the precursor tetrahydrothienopyridine series, wherein a 4-CF₃ affords optimal functional and pharmacokinetic properties. Interestingly, the inactive S-enantiomer of a related tetrahydroisoquinoline analog of AMG2850 positions this trifluorophenyl in the opposite direction, away from Trp798, while the corresponding R-enantiomer has a reported TRPM8 IC₅₀: 56±24 nM (derivatives 87-88 reported by Tamayo et al.). Moreover, Leu841 (S4) (distance: 4.2 Å, not shown) and the backbone of Arg842 (distance: 3.3 Å, not shown) interact with the trifluorophenyl CF₃ group of AMG2850 via a hydrophobic interaction. Tyr1005 (conserved residue, TRP helix) forms strong hydrophobic interactions with the 5,6,7,8-tetrahydro-1,7-naphthyridine, with a distance of 4.4 Å. This residue (Tyr1004 in TRPM8FA) hydrogen bonds to the icilin nitro group and WS-12 amide nitrogen, suggesting another common TRPM8 binding residue, in agreement with functional studies of menthol. Again, this interaction was consistent with the SAR data for this series, suggesting the importance of hydrophobic interactions between the ion channel and the azatetrahydroquinoline. Modifications including removal of the aromatic ring, expansion of the piperidine by one carbon (to an azepine), and conformational restriction via methyl substitution at the 8-position, results in significant loss or negates TRPM8 activity (derivatives 32, 72 and 86 reported by Horne et al. and Tamayo et al.), which may disturb this hydrophobic interaction. Alternatively, these analogs may disrupt the hydrogen bond between Arg842 and the urea carbonyl of AMG2850, resulting in a dramatic loss of TRPM8 activity.

On the opposite side of the molecule, it was observed that Asp781 (S2) interacts with the trifluoropropanyl CF₃ via hydrogen bonding with a distance of 3.4 Å. Similar to the previously discussed interactions, this interaction was consistent with SAR studies of this region on the azatetrahydroquinolinone and tetrahydroisoquinoline scaffolds, where the trifluoropropanyl group affords high TRPM8 potency. Together with hydrogen bonding interactions with Arg842 and Trp798, these orient AMG2850 in a T-shape conformation, with either ends of the molecule spanning the upper portion of the VSLD pocket, vs. the extended conformation seen with (−)-menthyl Compound 1. On the other hand, the azatetrahydroquinoline ring of AMG2850 penetrates deeply into the cavity, towards the TRP helix, similar to the biphenyl of Compound 1. Moreover, it was observed that Leu778 forms strong hydrophobic interactions with trifluoropropanyl CF₃ with a distance of 3.6 Å (not shown), similar to the (−)-menthyl of Compound 1. These results were consistent with that of the free energy decomposition using the MM/GBSA method (FIG. 3D).

Notably, from the MD studies, no direct interactions was found between the two antagonists studied and the Ca²⁺ binding residues, which could point to a possible structure-based explanation of their ability to function as antagonists. Interactions were observed between AMG2850 and Arg842, which could in theory reinforce the tetrahedral Ca²⁺ complex in human TRPM8 via Asp802, similar to that seen in the human TRPM4 structure (corresponding TRPM4 residues are Arg905 and Asp868). Such interactions with Arg842 could work to destabilize the bound Ca²⁺, resulting in channel inhibition. On the other hand, the TRPM8FA (avian) structure does not depict the corresponding residue Arg841 as interacting with Ca⁺ binding residues, but instead makes a direct interaction with the icilin carbonyl.

### Example 4—Structure-Based Design

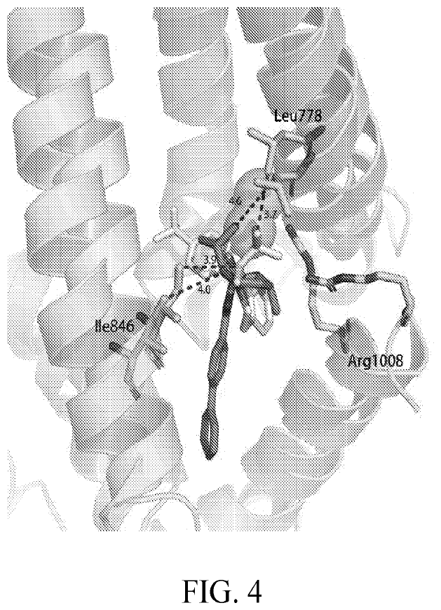

By superimposing the predicted binding modes of AMG2850 and (−)-menthyl Compound 1 from MD simulations, several important residues were observed that form a hydrophobic pocket within the VSLD, in the S2 and TRP helix (FIG. 4). Leu778 (S2) interacts with both the (−)-menthyl and trifluoropropanyl groups of Compound 1 and AMG2850. In the same vicinity, Arg1008 (TRP helix) forms a key hydrophobic interaction with the (−)-menthyl group. These interactions allow the aforementioned groups to superimpose in the VSLD, and suggests that the Compound 1 scaffold can be substituted with other lipophilic groups such as ring-expanded analogs of the core cyclohexyl ring, bi- and tricyclic rings, aromatic-containing groups and spiro-substituted bicyclics.

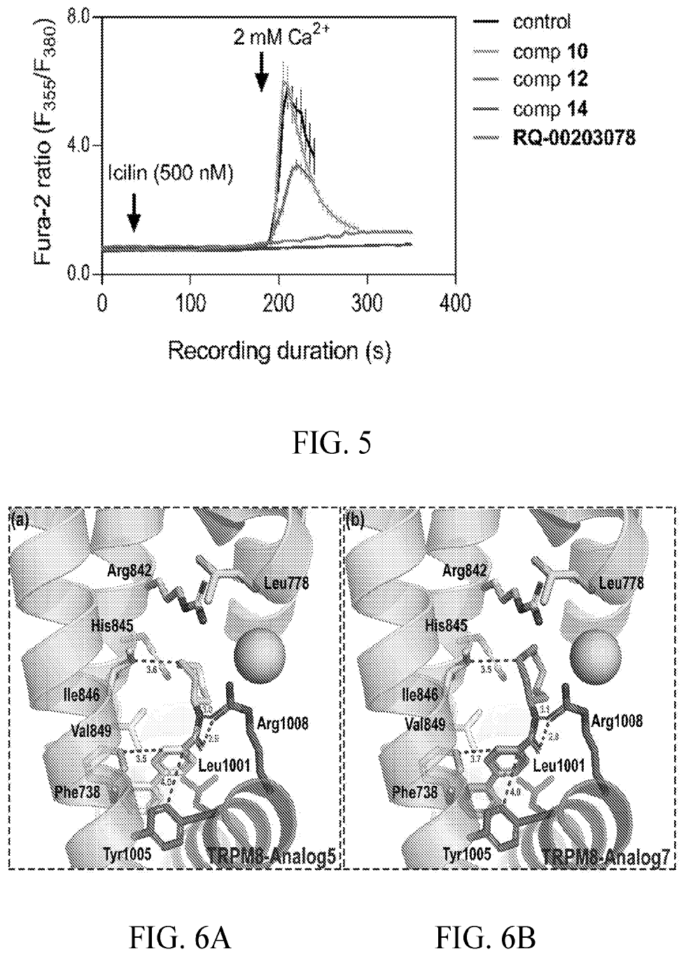

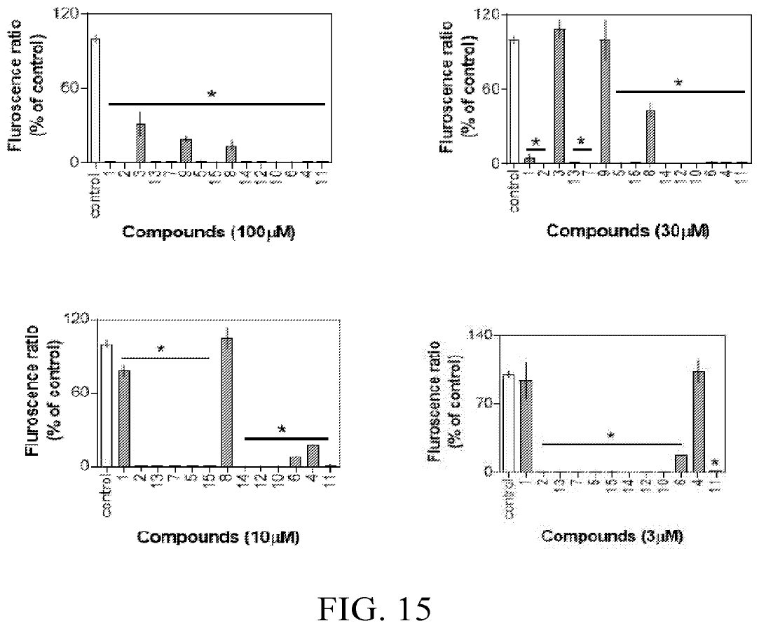

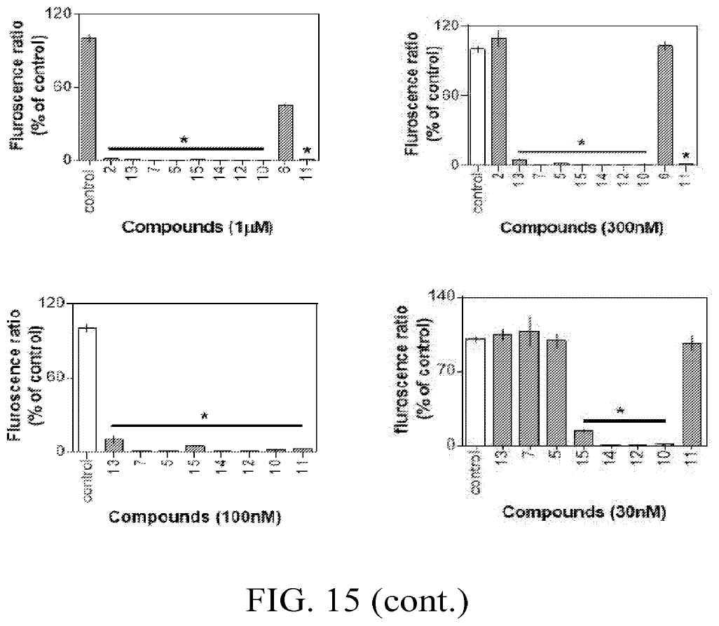

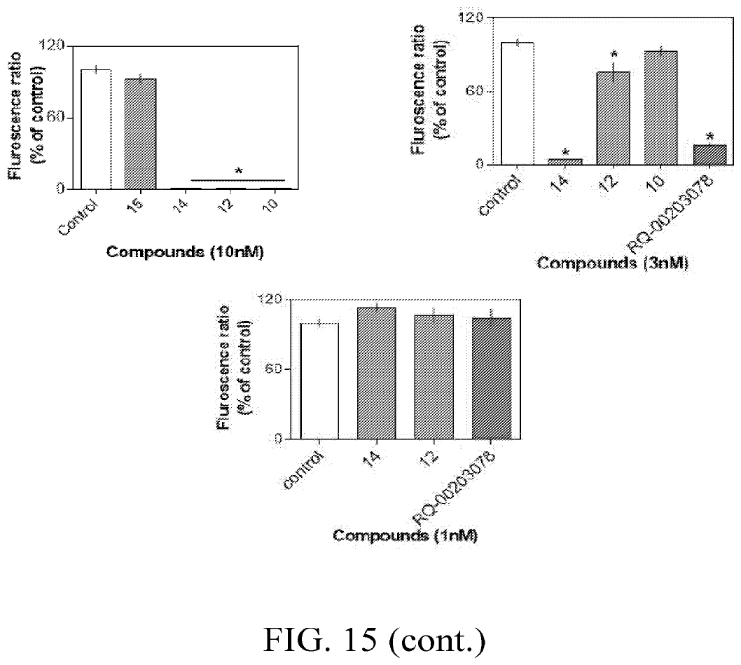

These substitutions were explored on the Compound 1 scaffold at human TRPM8 via ratiometric Ca²⁺ imaging (representative experiments are shown in FIG. 5, corresponding histograms are shown in FIG. 15), while retaining the biphenyl, as this was the optimal substitution for potent TRPM8 antagonist activity, from previous SAR studies. To this end, various lipophilic substitutions of the (−)-menthyl moiety of Compound 1 were prepared as shown in Scheme 1 below, via standard EDCI/HOBt-mediated amidations of commercially available substituted amines and [1,1′-biphenyl]-4-carboxylic acid I-1.

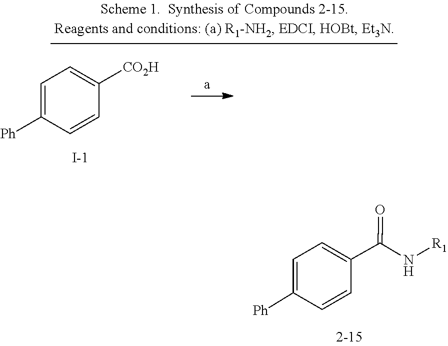

### Example 5—Structure-activity relationship (SAR) analysis of (−)-menthyl Compound 1

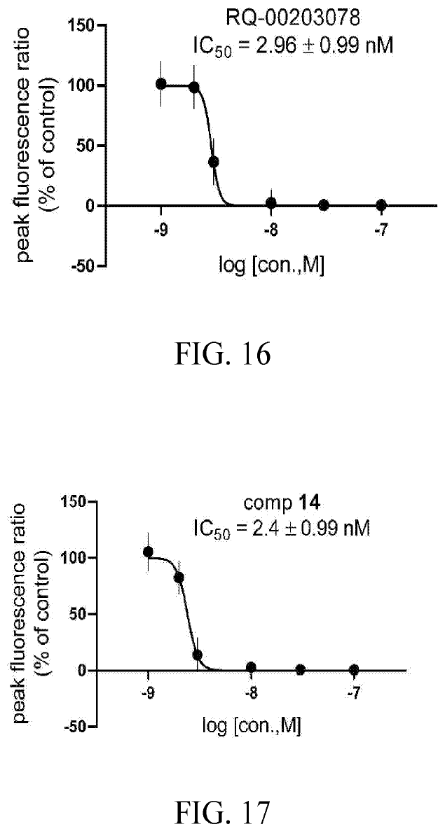

Dose-response studies of the prototypical icilin in this assay induces Ca²⁺ influx at an EC₅₀ of 74±3 nM (FIG. 13) and appears to be more potent than the previously-reported values lying within 120-200 nM range, but this could very well be due difference in cell type (CHO vs HEK cells) or species-dependence variation in icilin sensitivity (human versus mouse). In the present studies, icilin-evoked Ca²⁺ signals began to plateau at 500 nM. RQ-00203078 was used as a positive control as well as a reference standard for the SAR studies, due to its potent TRPM8 antagonist activity. In the assays, RQ-00203078 inhibits icilin-evoked Ca²⁺ signals at an IC₅₀ of 2.96±0.99 nM (FIG. 16), which is lower than the value (˜8 nM) previously reported against 30 μM menthol-evoked Ca²⁺ influx in HEK cells expressing hTRPM8.

For the presently-synthesized compounds, IC₅₀ values are shown in Table 1, and expressed as the concentration required to produce half-maximal inhibition of icilin-stimulated Ca²⁺ influx. (−)-menthyl Compound 1, was also resynthesized using methodology reported previously to serve as an additional standard for the biochemical assays at hTRPM8, due to its potent antagonist activity at rTRPM8 in calcium flux assays (IC₅₀ vs. 20 μM menthol, 20±2 nM; IC₅₀ vs. 0.25 μM menthol, 50±10 nM) and weak intrinsic (agonist) activity at hTRPV1 and rTRPA1. Interestingly, Compound 1 showed dramatically reduced potency (IC₅₀ of 16±1 μM) in the assays in HEK-293 cells transfected with human TRPM8 (Table 1). Differences in observed IC₅₀ values in the calcium flux assay at human vs. rat TRPM8 suggests species differences among TRPM8 orthologs for this compound. Species differences are known to exist between different TRPM8 orthologs despite high sequence conservation. Additional studies at rTRPM8 could be performed on all analogs of this series, to determine whether similar potency shifts are observed. Although human TRPM8 is highly homologous to the rat ortholog, with 96% sequence identity in the VSLD domain, pore domain and TRP helix (residues 734-1009, calculated by Clustal Omega, data not shown), there are a small number of different amino acid residues, possibly accounting for the discrepancy seen between these orthologs in the calcium flux assay. One such residue at position 1007, in the TRP helix, is situated in the binding pocket (serine at hTRPM8, asparagine at rTRPM8). Studies are ongoing to further characterize 1 and will be reported in due course.

Removing the chirality of Compound 1 to give isomeric mixture Compound 2 increases hTRPM8 activity by 30-fold (Table 1) in the presently-described assays (hTRPM8 IC₅₀: 468±1 nM) relative to Compound 1, indicating the mixture contains isomers with a more optimal stereochemical arrangement at the C₁, C₂ and C₅ positions than Compound 1. Relative to menthol isomeric mixture Compound 2, removal of the C₅ methyl and C₂ isopropyl groups (as in Compound 3) and conformational restriction of the core cyclohexyl ring (as in Compound 4) affords reduced TRPM8 potency, in the μM range, highlighting the entire (−)-menthyl scaffold as well as the conformational flexibility of the cyclohexyl ring, and possibly displacing hydrophobic interactions with Leu778 and Arg1008, as suggested by our MD studies.

TABLE 1
IC₅₀ values of Compounds in nM, determined by cellular Ca²⁺ flux assays in HEK-293
cells stably expressing hTRPM8 and represented as mean ± SEM from three independent
experiments. IC₅₀ is defined by concentration of the compounds that inhibit 50% of icilin-evoked
Ca²⁺ entry.

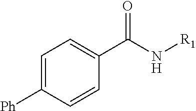

| | | |
|---|---|---|
| | | Functional Activity hTRPM8 |
| Cmpd | R₁ | potency IC₅₀ ± SEM |

| | | |
|---|---|---|
| 1 | 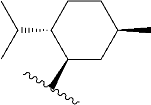 [SMILES: *[C@@H]1C[C@H](C)CC[C@H]1C(C)C] | 16 ± 1 μM |
| 2 | 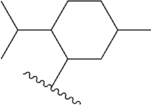 [SMILES: *C1CC(C)CCC1C(C)C] | 468 ± 1 nM |
| 3 | 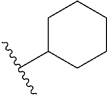 [SMILES: *C1CCCCC1] | 49 ± 1 μM |
| 4 | 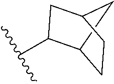 [SMILES: *C1CC2CCC1C2] | 7.1 ± 0.9 μM |
| 5 | 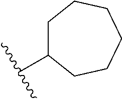 [SMILES: *C1CCCCCC1] | 52 ± 1 nM |
| 6 | 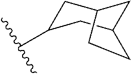 [SMILES: *C1CC2CCC(C1)C2] | 692 ± 1 nM |
| 7 | 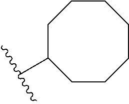 [SMILES: *C1CCCCCCC1] | 62 ± 1 nM |
| 8 | 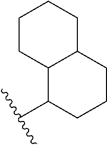 [SMILES: *C1CCCC2CCCCC12] | 21 ± 1 μM |
| 9 | 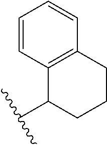 [SMILES: *C1CCCc2ccccc21] | 55 ± 1 μM |
| 10 (diastereomeric mixture) | 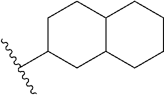 [SMILES: *C1CCC2CCCCC2C1] | 6 ± 1 nM |
| 11 | 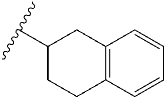 [SMILES: *C1CCc2ccccc2C1] | 52 ± 1 nM |
| 12 (isolated diastereomer) | 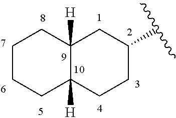 [SMILES: *[C@@H]1CC[C@@H]2CCCC[C@@H]2C1] | 1.4 ± 1.0 nM |
| 13 | 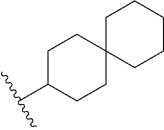 [SMILES: *C1CCC2(CCCCC2)CC1] | 40 ± 1 nM |
| 14 | 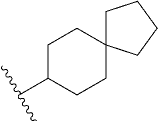 [SMILES: *C1CCC2(CCCC2)CC1] | 2.4 ± 1.0 nM |
| 15 | 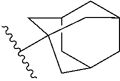 [SMILES: *C12CC3CC(CC(C3)C1)C2] | 16 ± 1 nM |
| RQ-00203078 | | 3 ± 1 nM |
| icilin | EC₅₀ | 74 ± 3 nM |
| (—)-menthol | EC₅₀ | 5.7 ± 0.72 μM |

On the other hand, ring expanded analogs (cycloheptyl Compound 5 and cyclooctyl Compound 7) improve hTRPM8 potency by 8-9 fold relative to Compound 2, with IC₅₀ values of 52+1 nM and 62+1 nM, respectively, suggesting the ability to capture additional interactions in the pocket. In order to rationalize these results, Compound 5 and 7 were docked into the hTRPM8 homology model (FIG. 6). For both Compounds, the biphenyl ring was positioned between the Si and S4 helices and TRP helix, similar to Compound 1. The biphenyl ring of both analogs forms strong hydrophobic interactions with Val849 (conserved, S4, 3.8/3.8 A), the same interaction seen for the Compound 1 biphenyl, in addition to Phe738 (conserved, Si, 3.5/3.7 Å), Leu1001 (conserved, TRP helix, 3.7/3.7 Å) and menthol- and icilin-sensitive Tyr1005 (conserved, TRP helix, 4.0/4.0 Å). The 1-phenyl group forms edge-to-face 7L-7L stacking with both Phe738 and Tyr1005. These additional interactions may contribute to the enhanced potency of these analogs in the Ca²⁺ flux assay, versus Compound 1. Interestingly, the 5,6,7,8-tetrahydro-1,7-naphthyridine of AMG2850 forms hydrophobic contacts with Tyr1005 (FIG. 3), in the same vicinity as the 1-phenyl of Compound 1 (FIG. 4). This reinforces the notion of Tyr1005 as a common small molecule binding residue. Both the amide NH and carbonyl O of Compounds 5 and 7 interact with Arg1008 (conserved, TRP helix) with strong hydrogen-bond interactions, possibly driving its potent functional activity; vs. that seen in Compound 1 where this residue only forms hydrophobic contacts with the (−)-menthyl moiety.

These results suggest that Arg1008 is a key residue for the recognition of both Compound 1 and its analogs. In addition, the cycloheptyl and cyclooctyl moieties of both ligands approached three residues with similar hydrophobic interactions, including voltage- and icilin-sensitive Arg842 (conserved, S4, 4.2/4.0 Å), icilin-sensitive His845 (conserved, S4, 3.7/4.0 Å), and Ile846 (conserved, S4, 3.6/3.5 Å), located on the opposite side of the VSLD as Leu778 and Arg1008.

Together, these results suggest that the orthosteric site is able to accommodate larger hydrophobic substitutions than that seen in Compound 1, with a number of other lipophilic residues as mentioned above, while the biphenyl provides an anchoring interaction towards the floor of the cavity.

Similar to Compounds 3 and 4, conformational restriction of Compound 5 (to bicyclo[3.2.1]octan-3-yl, Compound 6) results in a sharp decrease in potency, possibly due to the loss of multiple H-bond contacts to Arg1008. Exploration with bicyclic and tricyclic ring replacements were carried out to further probe the lipophilic space around our series. Decahydronapthalen-1-yl, Compound 8, results in a significant drop in hTRPM8 potency, relative to Compounds 5 and 7, in the μM range, indicating that the pocket is of a discriminate volume; similarly, tetrahydronaphthalen-1-yl (Compound 9) suffers a 3-fold loss of potency vs. Compound 8, again suggesting the need for flexibility at this region of the scaffold.

In striking contrast, decahydronaphthalen-2-yl (Compound 10), a mixture of two isomers, enhances TRPM8 potency by 3,500-fold relative to Compound 8 (IC₅₀: 6±1 nM), and has 10-fold higher potency than both the cycloheptyl (Compound 5) and cyclooctyl (Compound 7) analogs, suggesting the decahydronapthalen-2-yl ring occupies the lipophilic pocket to maximize available hydrophobic interactions. Consistent with the SAR trends seen with Compounds 8 and 9, substitution with a tetrahydronaphthalen-2-yl group (Compound 11) decreases hTRPM8 potency by 9-fold, though retains nM potency at hTRPM8 with an IC₅₀: 52±1 nM. Separating out one of the isomers of Compound 10 via chromatography to give Compound 12, gratifyingly affords potency in the single digit nM range, with an IC₅₀: 1.4±1.0 nM, 4-fold higher than the diastereomeric mixture. It is possible that Compound 10 forms multiple contacts with residues within the VSLD to give high hTRPM8 potency, similar to the single isomer Compound 12. LC-MS analysis of Compounds 10 and 12 indicated that Compound 10, a mixture of two diastereomers, is comprised of >85% of the Compound 12 isomer, thus accounting for its potent IC₅₀ value.

The relative stereochemical configuration of Compound 12 was established by 1H, ¹³C, gCOSY, NOESY, gHSQCAD, gHMBCAD and gH2BC (Table 2). Analysis of NMR data including comparison of decalin ring proton and carbon chemical shifts to literature values indicates that the compound is a cis-decalin with the biphenyl amide moiety in the 2-position.

Analysis of NOESY data indicates that the amide group is trans to the decalin bridge protons (see structure below, absolute stereochemistry cannot be established).

TABLE 2
Assignment of ¹H- and ¹³C-NMR chemical shifts of N-((2SR,9RS,10SR)-
decahydronaphthalen-2-yl)-[1,1′-biphenyl]-4-carboxamide (Compound 12).

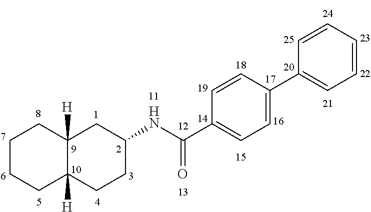 [SMILES: O=C(N[C@@H]1CC[C@@H]2CCCC[C@@H]2C1)c1ccc(-c2ccccc2)cc1]

| Atom # | ¹H (ppm) | ¹³C (ppm) |
|---|---|---|
| 1 | H1: 1.68 (m, 1H), H1′: 1.59 (m, 1H) | 32.72 |
| 2 | 4.01 (tdt, J = 12,8.3,4.3 Hz, 1H) | 49.9 |
| 3 | H3: 1.84 (br d, J = 12.9 Hz, 1H), H3′: 1.36 (m, 1H) | 27.9 |
| 4 | H4: 1.58 (m, 1H), H4′: 1.66 (m, 1H) | 30.85 |
| 5 | H5: 1.57 (m, 1H), H5′: 1.26 (m, 1H) | 25.72 |
| 6 | H6: 1.74 (m, 1H), H6′: 1.27 (m, 1H) | 26.82 |
| 7 | H7: 1.30 (m, 1H), H7′: 1.43 (m, 1H) | 20.93 |
| 8 | H8, H8′: 1.50-1.56 (m, 2H) | 31.74 |
| 9 | H9: 1.91 (m, 1H) | 35.0 |
| 10 | 1.68 (m, 1H) | 34.9 |
| 11 | 6.12 (br d, J = 8.1 Hz, 1H) | — |
| 12 | — | 166.3 |
| 13 | — | — |
| 14 | — | 133.7 |
| 15,19 | H15,19: 7.85 (d, J = 8 Hz, 2H) | 127.4 |
| 16,18 | H16,18: 7.64 (t, J = 8.1 Hz, 2H) | 127.16 |
| 17 | — | 144.0 |
| 20 | — | 140.1 |
| 21,25 | H21,25: 7.61 (d, J = 7.6 Hz, 2H) | 127.2 |
| 22,24 | H22,24: 7.46 (t, J = 7.5 Hz, 2H) | 128.9 |
| 23 | H23: 7.39 (t, J = 7.4 Hz, 1H) | 127.9 |

The structure was determined to be cis-decalin because the carbon and proton chemical shifts are more consistent with that configuration (see Dodziuk et al.). Cis-decalin bridge carbons (C_{9,10}) resonate at 36.1 ppm, whereas those in trans-decalin resonate at 42.5 ppm. Furthermore, the bridgehead protons resonate at lower ppm in the trans-decalin than that observed for this structure (compare 1.91, 1.68 ppm vs. 0.87 ppm for trans-decalin). Also see, Browne et al.

The relative stereochemistry of the amide moiety was more challenging to ascertain. NOESY data was consistent with the proposed structure; however, the severe chemical shift overlap, particularly with H₁₀ and H₁, rendered unambiguous assignment impossible. NOESY data did establish that H₉ (1.91 ppm) and H₂ (4.01 ppm) were on the same face. This result coupled with the chemical shift data was consistent with a cis-decalin substituted in the trans-2-position with a biphenyl carboxamide.

Spiro-substituted bicyclic replacement of the (−)-menthyl group of Compound 1 was then probed. Spiro [5.5]-undecan-3-yl (Compound 13) retained similar potency as Compounds 5 and 7 with an IC₅₀: 40±1 nM, though not in the high nM range as decahydronapthalen-1-yl (Compound 10 and 12), suggesting the complementary region of the receptor cavity is of a fixed volume and cannot tolerate a spiro-arrangement of the two cyclohexyl rings.

Ring contraction of Compound 13 by one carbon (to spiro[4.5]decan-8-yl, Compound 14) recapitulates the potent activity of decahydronapthen-1-yl isomer (Compound 12) (IC₅₀: 2.4±1.0 nM), with comparable activity as the highly potent TRPM8 antagonist RQ-00203078 in the assays. Spiro[4.5]decan-8-yl (Compound 14) was of similar potency as Compound 12, further suggesting the constrained or discerning nature of the pocket. The binding site for this series could be determined unequivocally using structural biology techniques such as cryo-EM or X-ray crystallography to produce a ligand bound structure of human TRPM8, coupled with mutagenesis studies. Currently, the structure of human TRPM8 is not known. Notably, Compound 14 shares several common structural features with other potent TRPM8 antagonists reported in the scientific and patent literature. Both a spiro group (spiro-isoxazoline) and biphenyl aromatic arrangement are also seen in highly potent benzimidazole-based TRPM8 antagonist JNJ41876666/TC-I 2014 (cTRPM8 IC₅₀ (icilin): 0.8 nM). Another TRPM8 antagonist reported by Glenmark Pharmaceuticals, (R)-(−)-10e (hTRPM8 IC₅₀ (⁴⁵Ca²⁺ uptake): 8.9 nM), contains a 3,4-dihydrospiro[chromene-2,4′-piperidine. Similarly, Raqualia Pharma reported a series of azaspiro TRPM8 antagonists containing ring systems such as 1-oxa-3-azaspiro[4.5]decan-2-ones, 1,3-diazaspiro[4.5]decane-2,4-diones and 1-oxa-3-azaspiro[4.5]decane-2,4-diones, linked via a ketone, to phenyl rings para substituted with aromatic and heterocyclic rings.

A dose-response curve of Compound 14 was then constructed from the data shown in FIG. 15. Compound 14 was selected for further characterization, given its lack of chiral centers unlike Compound 12. As shown in FIG. 17, compound 14 dose-dependently inhibited icilin-induced Ca²⁺ influx with an IC₅₀=2.4±1.0 nM and at higher concentrations, nearly abolishes the Ca²⁺ influx (FIGS. 15 and 17).

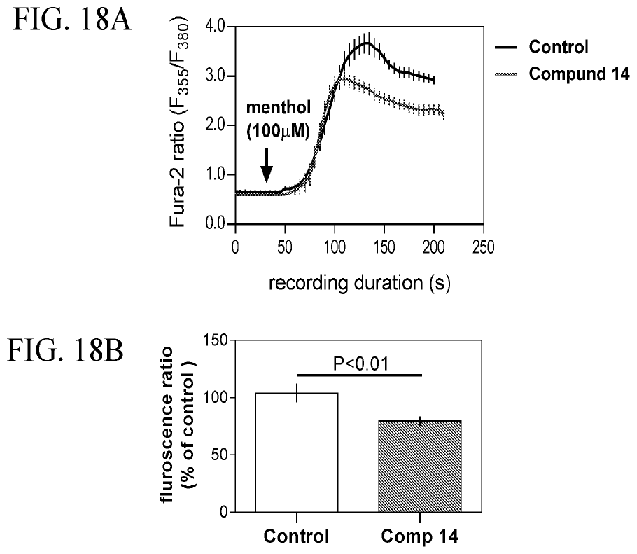

To determine the ability of Compound 14 to inhibit menthol in the Ca²⁺ flux assay, the menthol response was first evaluated in HEK-293 cells stably expressing hTRPM8 and found an EC₅₀˜6 μM (FIG. 14). The observed menthol potency was comparable to the values previously reported for hTRPM8 in similar calcium imaging of HEK293 cells. The lowest concentration at which Compound 14 was still able to cause significant reduction in icilin-evoked TRPM8-mediated Ca²⁺ entry was 3 nM (FIG. 17). Compound 14 was tested at the same lowest (i.e. 3 nM) concentration against the maximal (100 μM) menthol-evoked TRPM8-mediated Ca²⁺ entry (Figure S6a). As can be seen in FIG. 18B, Compound 14 at 3 nM was also able to significantly (P<0.01) reduce TRPM8-mediated Ca²⁺ entry triggered by menthol. These studies provide additional support for exploring the effects of Compound 14 in this assay using menthol as an agonist, in addition to our SAR studies carried out with icilin.

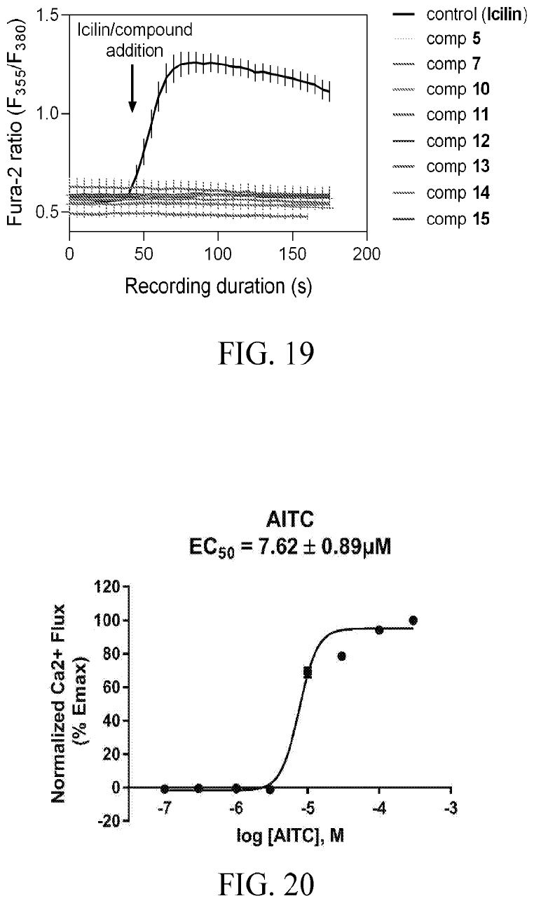

Adamantyl (Compound 15) affords slightly lower potency than Compound 14, with an IC₅₀: 16±1 nM, indicating that tricyclic rings are tolerated, as well as bicyclics (Compound 10-14). Altogether, the antagonist activity of Compounds 10, 12, 14 and 15 in the Ca²⁺ flux assay, with IC₅₀ values in the ≤10 nM range in human TRPM8-transfected cells, exceeds or is comparable to the Ca²⁺ flux activity of reported TRPM8 antagonists with in vivo activity, including PF-05105679 (Pfizer, Phase 1, hTRPM8 IC₅₀=181 nM), AMG 333 (Amgen, Phase 1, hTRPM8 IC₅₀=13±4 nM), AMG2850 (Amgen), RQ-00203078 (RaQualia Pharma), AMTB (Bayer), arylglycine derivative 12 and vinylcycloalkyl-substituted benzimidazole 25 (Janssen), DFL23448 and DFL23693, M8-B, M8-An, tryptophan derivative 14, and others. None of the Compounds with hTRPM8 IC₅₀ values ≤100 nM in the Ca²⁺ flux assay (Compounds 5, 7, 10-15), were able to stimulate the channel alone at a single high concentration of 10 μM (FIG. 19), in agreement with results obtained in our antagonist assays.

### Example 6—In Vitro Selectivity Profiling at TRPA1 and TRPV1

The selectivity profile of the novel Compounds 5, 7, and 10-15 were next determined vs. two temperature-related TRP subtypes, hTRPA1 and hTRPV1. Many TRP ligands have inverse behavior, for example, the TRPM8 agonist menthol blocks TRPV1 and TRPA1, while activating TRPA1 at lower concentrations. Similarly, the TRPV1 agonist capsaicin is able to inhibit TRPM8-mediated responses. On the other hand, TRPV1 antagonists capsazepine, BCTC and SB-452533 and others exhibit dose-dependent blockade of TRPM8. These studies suggested ligand recognition overlaped between TRPM8, TRPA1 and TRPV1. Similar to TRPM8, TRPA1 is activated by cold temperatures in vitro and in vivo, though at slightly lower temperatures. Icilin also activates TRPA1 at μM concentrations in the Ca²⁺ flux assay.

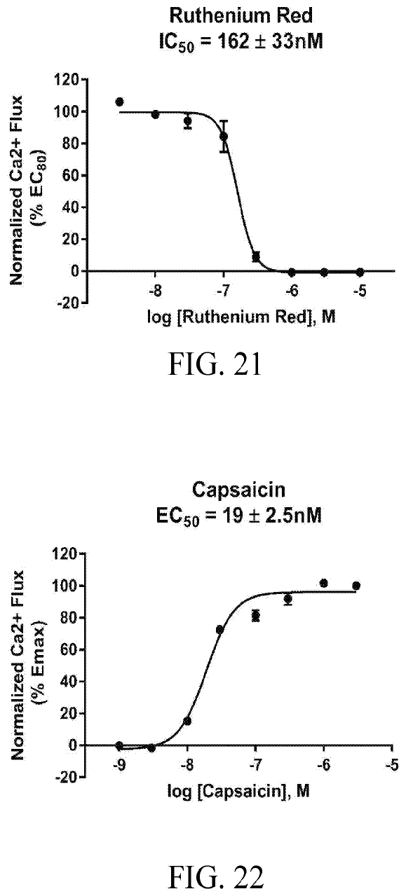

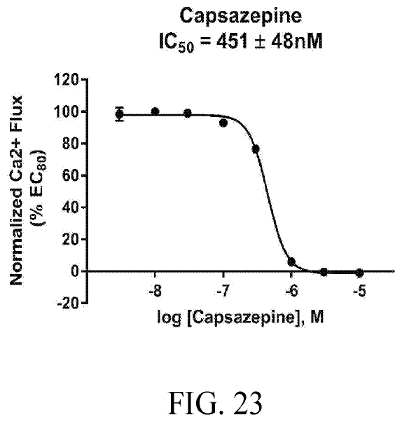

In the present TRPA1 Ca²⁺ flux assays, dose-response studies of the TRPA1 agonist AITC affords an EC₅₀ of 7.62±0.89 μM (FIG. 20), in agreement with previous studies. The TRPA1 antagonist ruthenium red, used as a standard and positive control in the assays, dose-dependently blocks AITC (10 μM)-activated [Ca²⁺]ᵢ release at an IC₅₀ of 162±33 nM (dose-response curve shown in FIG. 21), in the same range as Klionsky and colleagues, who report an IC₅₀ of 29±6 nM vs. the effect of 80 μM AITC. At TRPV1, the EC₅₀ and IC₅₀ values for the TRPV1 agonist capsaicin (EC₅₀ 19±2.5 nM, FIG. 22) and antagonist capsazepine (IC₅₀ 451±48 nM, FIG. 23) are similar to values reported by El Kouhen et al. Curves included in FIGS. 20-23 are listed at ±95% Confidence interval.

TABLE S1
TRPA1 Agonist Data (Percentage
Activation Normalized to Eₘₐₓ Control).
All data are normalized to the Eₘₐₓ
for the reference agonist AITC.

| | | % Activation |
|---|---|---|
| | Conc | Mean ± SEM |
| Compd | (nM) | (n = 5) |
| AITC | 100 | −0.8 ± 0.4 |
| | 300 | −0.5 ± 0.2 |
| | 1,000 | −0.5 ± 0.2 |
| | 3,000 | −1.2 ± 0.2 |
| | 10,000 | 68.8 ± 2.9 |
| | 30,000 | 78.5 ± 1.0 |
| | 100,000 | 94.2 ± 2.4 |
| | 300,000 | 100.0 ± 1.3* |
| DMSO control | 0.33% | 0.0 |

*n = 10

TABLE S2
TRPA1 Antagonist Data (Percentage
Inhibition Normalized to EC₈₀ Control).
All data are normalized to the EC₈₀
for the reference agonist AITC.

| | | % Activation |
|---|---|---|
| | Conc | Mean ± SEM |
| Compd | (nM) | (n = 5) |
| Ruthenium | 3 | −6.0 ± 1.2 |
| Red | 10 | 1.9 ± 2.4 |
| | 30 | 5.8 ± 4.6 |
| | 100 | 15.7 ± 9.8 |
| | 300 | 91.1 ± 2.9 |
| | 1,000 | 100.9 ± 0.1 |
| | 3,000 | 100.9 ± 0.7 |
| | 10,000 | 100.8 ± 0.3 |
| AITC EC₈₀ | 10,000 | 100 |
| DMSO control | 0.33% | 0.0 |

TABLE S3
TRPV1 Agonist Data (Percentage
Activation Normalized to Eₘₐₓ Control).
All data are normalized to the Eₘₐₓ
for the reference agonist Capsaicin.

| | | % Activation |
|---|---|---|
| | Conc | Mean ± SEM |
| Compd | (nM) | (n = 5) |
| Capsaicin | 1 | −0.2 ± 1.0 |
| | 3 | −1.7 ± 0.7 |
| | 10 | 15.2 ± 0.6 |
| | 30 | 72.4 ± 2.3 |
| | 100 | 81.4 ± 3.3 |
| | 300 | 91.8 ± 3.7 |
| | 1,000 | 101.6 ± 2.0 |
| | 3,000 | 100.0 ± 1.8* |
| DMSO control | 0.33% | 0.0 |

*n = 10

TABLE S4
TRPV1 Antagonist Data (Percentage
Inhibition Normalized to EC₈₀ Control).
All data are normalized to the EC₈₀
for the reference agonist Capsaicin.
TRPV1 Antagonist assay:
Capsazepine reference control

| | | % Activation |
|---|---|---|
| | Conc | Mean ± SEM |
| Compd | (nM) | (n = 5) |
| Capsazepine | 3 | 1.6 ± 4.2 |
| | 10 | 0.1 ± 1.3 |
| | 30 | 1.1 ± 1.7 |
| | 100 | 7.1 ± 2.2 |
| | 300 | 23.4 ± 2.5 |
| | 1,000 | 94.2 ± 1.1 |
| | 3,000 | 100.6 ± 0.3 |
| | 10,000 | 101.2 ± 0.5 |
| Capsaicin EC₈₀ | 100 | 100 |
| DMSO control | 0.33% | 0.0 |

Among the compounds with significant TRPM8 antagonist activity, all analogs with the exception of Compounds 5, 7, and 11 had negligible intrinsic (agonist) and antagonist activity in Ca²⁺ flux assays at both hTRPV1 and hTRPA1, at concentrations corresponding to approximately 2×their hTRPM8 IC₅₀ values, and at a 10-fold greater concentration than the former value. Specifically, none of the compounds showed greater than 25% activation at hTRPA1 and hTRPV1 when normalized to the Eₘₐₓ concentration of the reference agonists AITC (300 μM) and capsaicin (3 μM), at both concentrations tested. Similarly, none of these analogs were able to inhibit either subtype at greater than 25% when challenged with the EC₈₀ concentrations of AITC and capsaicin (10 μM and 0.1 μM, respectively). For example, even at a 17-fold higher concentration than its hTRPM8 IC₅₀ value of 6±1 nM, Compound 10 does not activate or inhibit hTRPA1 or hTRPV1. Similarly, Compound 12, 13, 14 and 15 do not affect hTRPA1- and hTRPV1-mediated responses at concentrations 21-, 25-, 13- and 19-fold higher than their hTRPM8 IC₅₀ values of 1.4±1 nM, 40±1 nM, 2.4±1.0 nM and 16±1 nM, respectively. Thus, many of the Compound in the present series are at least 10-fold selective for TRPM8 vs. related thermoTRPs TRPA1 and TRPV1.

TABLE 3
Intrinsic and antagonist functional activities of selected biphenyl
amides at TRPA1 and TRPV1 receptors, determined in Ca²⁺ flux assays

| | Concentration | hTRPA1 | hTRPA1 | hTRPV1 | hTRPV1 |
|---|---|---|---|---|---|
| | tested | % | % | % | % |
| | (x TRPM8 | Activation | Inhibition | Activation | Inhibition |
| Cmpd | IC₅₀) | (n = 3-5) ^{b} | (n = 3) ^{c} | (n = 3) ^{b} | (n = 3) ^{c} |
| 5, | 100 nM | 0.1 ± 0.7 | −2 ± 5 | 0.0 ± 1.0 | −3 ± 4 |
| hTRPM8 IC₅₀: | 1 μM | 0.8 ± 0.5 | 4 ± 5 | 0.6 ± 1.5 | 24 ± 1 |
| 52 ± 1 nM | | | | | |
| 7, | 100 nM | −1.0 ± 0.1 | −13 ± 4 | −1.9 ± 0.7 | −4 ± 4 |
| hTRPM8 IC₅₀: | 1 μM | −1.5 ± 0.9 | 0.2 ± 6 | −3.2 ± 0.6 | 14 ± 4 |
| 62 ± 1 nM | | | | | |
| 10, | 10 nM | 0.2 ± 0.4 | −8 ± 3 | 2.4 ± 0.8 | −13 ± 4 |
| hTRPM8 IC₅₀: | 100 nM | −1.1 ± 0.2 | −1 ± 10 | 2.3 ± 0.9 | 2 ± 3 |
| 6 ± 1 nM | | | | | |
| 11, | 100 nM | −0.1 ± 0.5 | 3 ± 6 | −1.8 ± 0.9 | −3.1 ± 0.6 |
| hTRPM8 IC₅₀: | 1 μM | −0.8 ± 0.6 | 4 ± 1 | −0.7 ± 1.5 | 13 ± 3 |
| 52 ± 1 nM | | | | | |
| 12, | 3 nM | −2.3 ± 1.0 | −0.3 ± 3 | 2 ± 1 | −12 ± 3 |
| hTRPM8 IC₅₀: | 30 nM | −2.1 ± 0.5 | 5 ± 9 | 3 ± 1 | 1.0 ± 2 |
| 1.4 ± 1.0 nM | | | | | |
| 13, | 100 nM | 0.2 ± 0.5 | −1 ± 5 | −1.0 ± 0.4 | −3 ± 4 |
| hTRPM8 IC₅₀: | 1 μM | −0.2 ± 1.1 | −7 ± 9 | 0.7 ± 0.2 | 5 ± 2 |
| 40 ± 1 nM | | | | | |
| 14, | 3 nM | −0.8 ± 0.4 | −6 ± 5 | 1 ± 1 | −9 ± 2 |
| hTRPM8 IC₅₀: | 30 nM | 0.1 ± 1.2 | −3 ± 2 | 1 ± 2 | −0.6 ± 5 |
| 2.4 ± 1.0 nM | | | | | |
| 15, | 30 nM | −2.6 ± 0.5 | −7 ± 5 | 1.0 ± 0.2 | −6 ± 5 |
| hTRPM8 IC₅₀: | 300 nM | −1.5 ± 0.4 | −7 ± 7 | 1 ± 2 | 3 ± 4 |
| 16 ± 1 nM | | | | | |

^{a} Data are normalized to the maximum response triggered by 500 nM icilin.
^{b} Data are normalized to the concentration corresponding to the Eₘₐₓ value of TRPA1 and TRPV1 agonists AITC (300 μM) and capsaicin (3 μM), respectively.
^{c} Data are normalized to the EC₈₀ of AITC (10 μM) and capsaicin (0.1 μM).

### Example 7—Whole-Cell Patch Clamp Electrophysiology of Compound 14

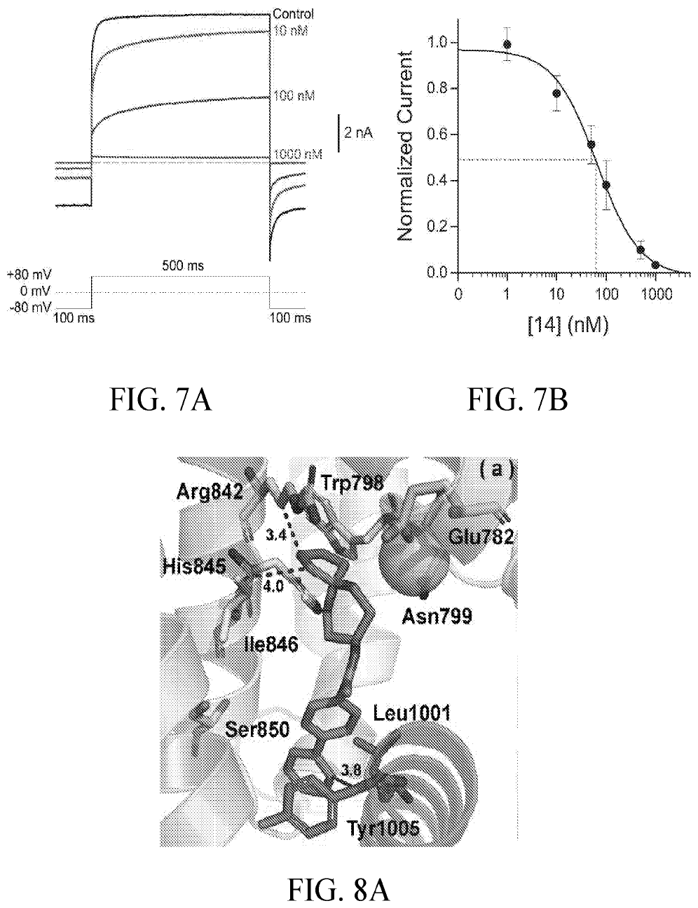

Whole-cell patch clamp electrophysiology was used to further investigate and validate the antagonist activity of Compound 14 at hTRPM8, using the cognate ligand menthol to stimulate the channel (FIG. 7). In patch clamp experiments, activation of TRPM8 by icilin in transfected HEK-293 cells or TRPM8-expressing Xenopus ooctyes is dependent on the presence of extracellular Ca²⁺, additionally, icilin activates the channel with highly variable latencies and marked desensitization, an effect not seen with menthol. Considering these things, we further explored the ability of Compound 14 to inhibit menthol-mediated responses at hTRPM8, as detailed in previous publications. In contrast to a Ca²⁺ and pH dependent icilin-evoked response in whole-cell recordings, menthol induces robust activation of TRPM8 in the voltage-clamp assay, allowing for better comparison studies. Considering these things, the ability of Compound 14 to affect currents induced by the agonist menthol was evaluated.

In agreement with Ca²⁺ flux studies using icilin, this Compound inhibits menthol-induced TRPM8 responses in a concentration-dependent manner, with an IC₅₀ of 64±4 nM, demonstrating that Compound 14 is a bona fide and functionally robust TRPM8 antagonist, with the ability to inhibit TRPM8 responses at nM concentrations, elicited by different chemical stimuli. The antagonist activity of Compound 14 surpasses a tryptamine-based antagonist by 6-fold in the same assay, with similar potency as P-lactam antagonists containing three labile ester groups. Although the IC₅₀ value of Compound 14 does not approach that of the well-known TRPM8 antagonist PBMC in this assay (IC₅₀: 0.4-0.6 nM), this benzyloxy-phenylmethylcarbamate analog of AMTB, containing labile ester and carbamate groups, appears to have limited utility as a chemical probe. PBMC gives variable responses in different behavioral models of neuropathic pain, and is unable to block cold sensitization in the CIPN model. Altogether, Compound 14 can overcome assay-specific nuances related to different chemical activators, demonstrating robust, highly potent antagonist activity in both the Ca²⁺ flux and whole cell patch clamp assay when challenged with both icilin and menthol. Compound 14 is well characterized pharmacologically, and has TRPM8 activity in a number of assays vs. other small molecule TRPM8 ligands, demonstrating its viability as a chemical tool to probe TRPM8-mediated pharmacology. As discussed below, Compound 14 interacts with Arg842 and Tyr1005, also shown to bind to icilin and menthol analog WS-12. Binding to these residues may suggest why Compound 14 is able to inhibit both icilin and menthol-mediated responses in Ca²⁺ flux and electrophysiology assays, respectively; however site specific mutagenesis studies could shed further insight.

### Example 8—Compound 14 and hTRPM8

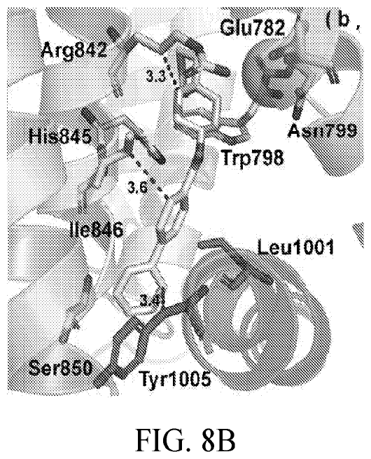

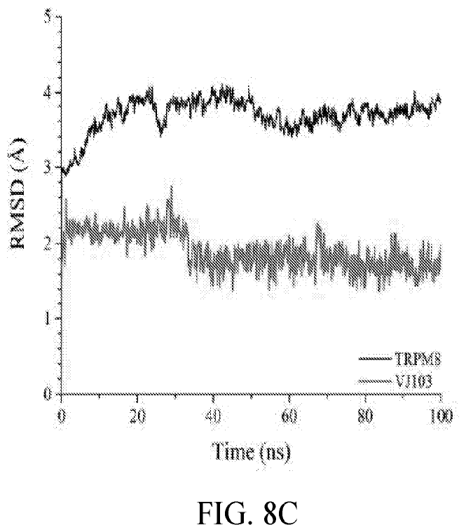

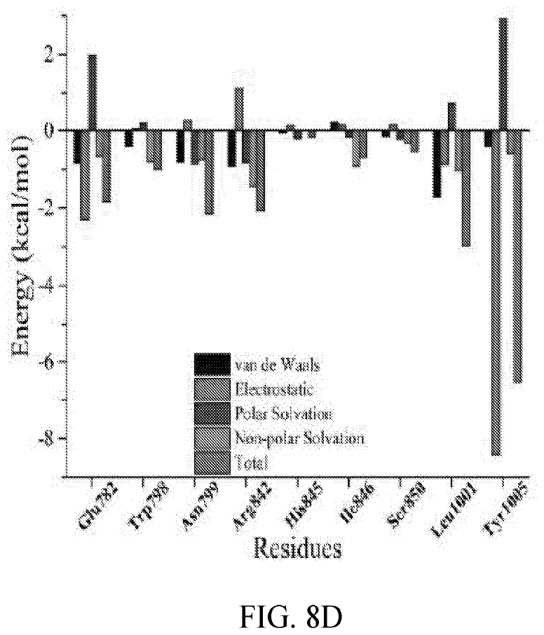

Experiments were next undertaken to rationalize the potent antagonist activity of Compound 14 in the context of the human TRPM8 homology model, by carrying out 100 ns MD simulations. Briefly, 100 ns MD simulations were carried out for the highly potent antagonist, spiro[4.5]decan-8-yl Compound 14 in hTRPM8. It was found that both the RMSD of hTRPM8 (about 3.9 Å) and Compound 14 (1.9 Å) kept stable after 30 ns as shown in FIG. 8C, indicating the time scale of 100 ns is reasonable.

As shown in FIG. 8A, the cyclopentyl of Compound 14 forms strong hydrophobic interactions with conserved Arg842 (S4, side chain, 3.4 Å) and Ile846 (S4, 4.0 Å) residues within the lower S4 helix, similar to the cycloheptyl and cyclooctyl of Compounds 5 and 7 (FIG. 6). The biphenyl contacts conserved Leu1001 (˜3.1 Å, not shown) and Tyr1005 (menthol-sensitive, 3.8 Å) residues in the TRP helix before MD simulation. FIG. 8B shows the binding pose of Compound 14 within TRPM8 after MD simulation. It was observed that the interactions between the biphenyl of Compound 14 and Ile846 (3.6 Å)/Leul001 (3.3 Å, not shown)/Tyr1005 (3.4 Å) kept stable during the MD, as shown in FIG. 8B. Ile846 shifts instead to make hydrophobic contacts with the biphenyl group, similar to Leu1001 and Tyr1005.

Interactions with Leu1001 and Tyr1005 are again consistent with the docked poses of Compounds 5 and 7, as discussed previously.

Ile846, Leu1001 and Tyr1005 anchor the biphenyl into the base of the VSLD, where it is more deeply buried than the biphenyl of Compound 1, again suggesting this as a plausible rationale for agonist-vs. antagonist-mediated behavior for these chemotypes.

Importantly, the hydrophobic interaction observed between Tyr1005 and the biphenyl is also seen in the AMG2850 post-MD pose. This residue also contacts WS-12 and icilin via polar interactions as discussed previously, reinforcing the notion of Tyr1005 as a common TRPM8 residue for ligand recognition. Unlike Compound 1 and WS-12, the aromatic-containing groups do not overlap in the VSLD.

Surprisingly, it was found that the spiro[4.5]decan-8-yl group maintains hydrophobic contacts with Arg842, though with the cyclohexyl ring. Together with Ile846, Leu1001 and Tyr1005, the energy decomposition of the key residues further supports the notion that these four residues may contribute greater than others to the recognition of Compound 14 in TRPM8, as shown in FIG. 8D. Arg842 represents another common residue for small molecule binding suggested from previous cryo-EM studies and the MD studies on AMG2850, on the opposite side of the VSLD vs. the menthol-binding site, approaching the bound Ca²⁺ ion and coordinating residues in the S2-S3 helices. This forces the molecule into an extended confirmation. This region of the VSLD orthosteric site (lower S2 helix, and S3-S4 helices) contains a number of hydrophobic as well as polar residues, and may preferentially accommodate larger hydrophobic substitutions on our biphenyl amide series such as those seen in our potent nanomolar antagonists/Compounds 5, 7 and 10-15 vs. the menthol-binding region in the Si and upper S2 helix, providing structural context for our experimentally observed SAR trends. Residues within this vicinity (Glu782, Arg842 (Arg841 in TRPM8FA) and His845 (His844 in TRPM8FA), among others) are reported to adopt multiple conformations, allowing TRPM8 to bind both WS-12 and icilin.

The presence of bulky residues such as Trp798 (S3) and His845 (S4), 3.6 and 3.8 A away from the spiro cyclohexyl group, may serve to sterically repulse the decahydronapthalen-1-yl and tetrahydronaphthalen-1-yl rings of Compounds 8 and 9, effectively decreasing their TRPM8 potencies to the μM range, vs. other analogs in our series with a greater degree of conformational flexibility at this region (as in Compounds 5 and 7) or a more optimal bicyclic arrangement (Compounds 10-14).

Interestingly, Compound 14 does not interact with Arg1008 (as is the case for Compounds 1, 5 and 7) or form hydrogen bonds, but instead forms extensive hydrophobic contacts within the binding cavity, similar to that seen in the recently reported structures of antagonists AMTB and JNJ41876666/TC-I 2014 bound to Parus major (parrot) TRPM8 (pmTRPM8) (PDB 606R and 6072, respectively). Comparison of the post-MD pose of Compound 14 with the JNJ41876666/TC-I 2014-complexed structure reveals the biphenyls of both antagonists superimpose within the VSLD, supporting our MD results, while the spiro-containing moieties are oriented oppositely in the pocket. Opposite orientations seen for the spiro[4.5]decan-8-yl of Compound 14 vs. the spiro-isoxazoline of JNJ41876666/TC-I 2014 are attributed to projection of either group off of an amide (trigonal planar geometry, 120°) vs. a conformationally rigid benzimidazole, respectively.

### Example 9—In silico selectivity profiling at TRPA1 and TRPV1

In order to rationalize the selectivity profile of Compound 14, that Compound was docked into the cryo-EM structures of rTRPV1 (PDB 3J5R) and hTRPA1(PDB 3J9P), and these poses were compared to the pre-MD (docked) pose in the hTRPM8 homology model.

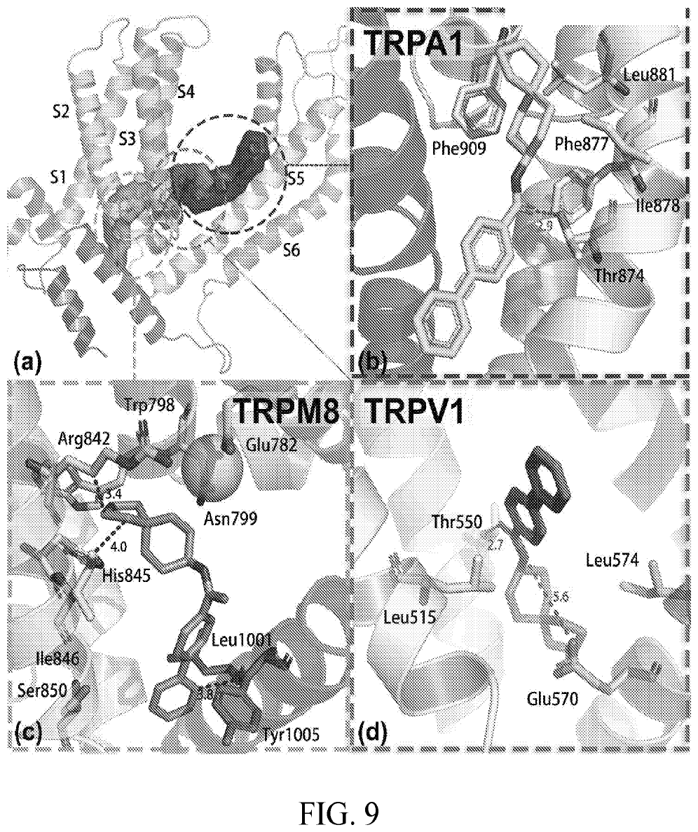

Interestingly, the binding pockets of TRPM8, TRPV1 and TRPA1 occupy different regions of the VLSD (S1-S4), the pore domain formed by S5-S6, and the pore helix (FIG. 9). Vanilloid ligands resiniferatoxin (RTX), a capsaicin-like homovanillyl ester antagonist, and the competitive antagonist capsazepine bind to TRPV1 residues in the S3 and S4 helices, and the S4-S5 linker; at 2.9 Å and 3.4 Å. Taken together with the liganded cryo-EM structure of avian TRPM8, whose binding pocket is defined within the S1-S4 helices and TRP helix, these structures suggested the absence of a central binding site for structurally disparate natural product ligands for both TRP subtypes, namely menthol and capsaicin, respectively. Rather, these molecules bind to different areas within the VSLD of each channel. While the structure of TRPA1 is not liganded (˜4 Å), cryo-EM along with mutagenesis studies of a key Phe909 residue suggested that the natural product agonist allyl isothiocyanate (AITC) and antagonist A-967079 bind in a site defined by residues in the S5, S6 and pore helix, again suggesting that prototypical TRP channel agonists from nature bind to different pockets on their respective subtypes.

In order to rationalize the selectivity of the compound, Compound 14 was docked to TRPA1 and TRPV1, and these results were compared to the docked pose (pre-MD) of Compound 14 at TRPM8 (docking energy of−9.9 kcal/mol at TRPM8). As shown in FIG. 9C, the docked pose shows Compound 14 forms extensive interactions with TRPM8. Several important residues contributed to the binding of Compound 14, including Trp798, Arg842, His845, Ile846, Leu1001, and Tyr1005. FIG. 9B shows the interactions between Compound 14 and TRPA1. The binding pocket of TRPA1 revealed a unique density within a pocket formed by S5, S6 and the first pore helix, including Phe909 (pore helix 1), Leu881 (S5), Ile878 (S5), Phe877 (S5), and Thr874 (S5), which is consistent with the reported binding site. By analyzing the interactions, it was found that Thr874 forms a hydrogen bond with Compound 14, while Phe909, Leu881, Ile878, and Leu871 contributed to the hydrophobic interactions. FIG. 9D shows the binding pose of Compound 14 in TRPV1. The pocket in TRPV1 was formed by five trans-membrane domains from two adjacent monomers, including S3, S4, the S4-S5 linker, S5 and S6. Several important residues within the binding pocket are shown in FIG. 9D, including Tyr511 (S3), Leu515 (S3), Leu574 (S4), Thr550 (S4), Arg557 (S4-S5 linker), Glu570 (S5 of the first monomer), and Leu672 (S6 of the second monomer). It was found that Thr550 forms a strong hydrogen bond with Compound 14, while Leu515 and Leu574 form hydrophobic contacts with Compound 14. The docking energies of Compound 14 at TRPM8, TRPA1, and TRPV1 were−9.9 kcal/mol,−7.1 kcal/mol, and−8.3 kcal/mol, indicating that Compound 14 should preferentially bind to TRPM8. From the high throughput FLIPR assays in Table 3, Compound 14 was modestly selective (13-fold) for TRPM8 vs. TRPA1 and TRPV1.

Metabolic stability testing of Compound 14 was carried out in mouse liver microsomes to determine its suitability as a TRPM8 chemical probe in vivo. Compound 14 has a half-life (t1/2) of 30 min in mouse liver microsomes, and an intrinsic clearance (CLᵢₙₜ) of 0.0455 mL/min/mg; indicating sufficient metabolic stability to conduct the behavioral assays (Table S5). Compound 14 may undergo CYP450 hydroxylation of the spiro or aromatic rings, leading to t_{1/2}=30 min.

TABLE S5
Stability of Compound 14 in mouse liver microsomes.

| Compd | % Remaining of Initial (n = 1) | | | | | | CLᵢₙₜ ^{a} |
|---|---|---|---|---|---|---|---|
| 14 | 0 | 10 | 20 | 30 | 60 | Half-life | (mL/min/mg |
| testosterone | min | min | min | min | min | (min) | protein) |
| | 100 | 78 | 57 | 47 | 31 | 30 | 0.0455 |
| | | | | | | 3.7 | 0.372 |

^{a} Intrinsic clearance (CLᵢₙₜ) was calculated based on CLᵢₙₜ = k/P, where k is the elimination rate constant and P is the protein concentration in the incubation.

### Example 10—In Vivo Analysis

Wet Dog Shakes (WDS) assay. To determine whether the observed ability of Compound 14 to potently inhibit TRPM8 activity in vitro also translates in TRPM8-dependent antinociceptive activity in vivo, a “wet-dog shakes” (WDS) assay was performed. In this assay, icilin (10 mg/kg intraperitoneally i.p.), was injected in mice treated with Compound 14 or vehicle. In mice, TRPM8 is known to share high homology with rat TRPM8, that has been used for in vitro assays. Icilin injections in mice produce vivid and quantifiable TRPM8-mediated shaking behaviors. Compound 14 (1, 30 and 50 mg/kg) or its vehicle were administered subcutaneously (s.c.) 30 minutes before icilin injection.

Cold Allodynia. Increasing evidence suggest that TRPM8 μlays a role in mouse models of chemotherapy-induced neuropathic pain evoked by oxaliplatin (OXP), a condition mimicking cold hypersensitivity provoked by chemotherapy-induced peripheral neuropathy (CIPN). Both acute and chronic OXP-induced cold hypersensitivity have been reproduced in rats and correlated with TRPM8 expression and function. Mizoguchi et al. reported that, in a rodent model, acute cold allodynia after OXP injection was alleviated by the TRPM8 blockers N-(2-aminoethyl)-N-[4-(benzyloxy)-3-methoxybenzyl]-N′-(1S)-1-(phenyl) ethyl] urea and TC-I 2014. According to these findings, the effect of Compound 14 was investigated in an OXP-induced cold allodynia model, using acetone for cooling stimulation. Considering that the cold pain threshold is increased from ≈12° C. to ≈26° C. in OXP-treated patients, acetone stimulation is considered to evoke pain in OXP-treated mice.

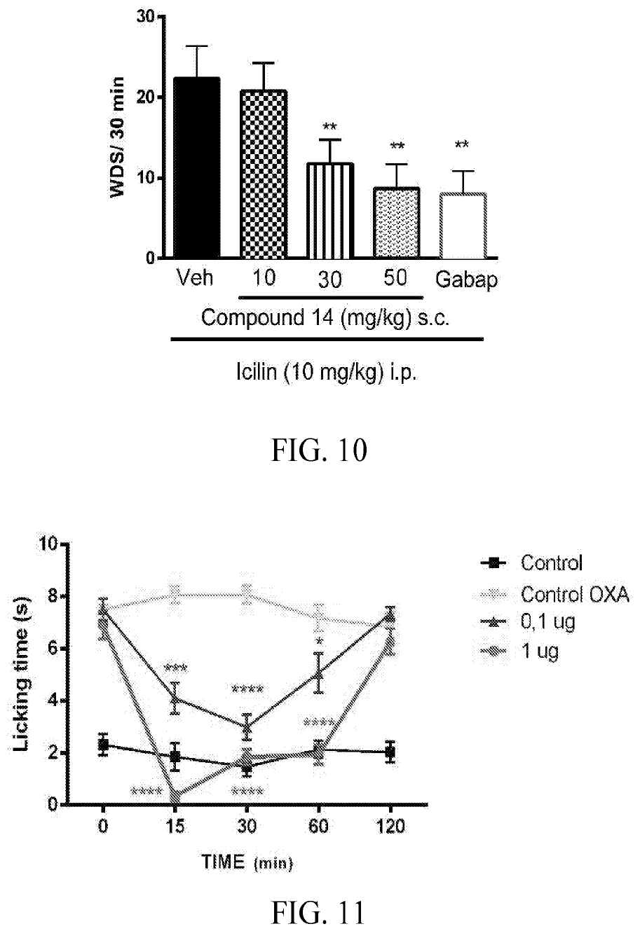

As shown in FIG. 10, Compound 14 produces marked, dose-dependent antinociceptive behavior. At the highest dose of 50 mg/kg, Compound 14 inhibits WDS like cold hypersensitivity in mice with an efficacy comparable to that observed with a standard analgesic dose of gabapentin (25 mg/kg, s.c.)

These results link the antagonist activity of Compound 14 in both the Ca²⁺ flux and patch-clamp assay to TRPM8 mechanistically, in a whole animal model, since icilin-induced WDS behaviors are not reproducible in TRPM8 null mice. This data demonstrates a key attribute for a chemical or pharmacological probe molecule.

ThermoTRPs, including TRPM8, TRPA1, TRPV1, TRPV2 and TRPV4, are described in the literature as potential new targets for treating chemotherapy-induced peripheral neuropathy (CIPN). Defects in sensory perception to cold are manifested as cold allodynia, defined as pain experienced from a normally non-painful cold stimulus; and cold hyperalgesia, a heightened pain response from a painful cold stimulus, among others. Such cold deficiencies are seen clinically in chemotherapy-induced allodynia, neuropathic pain, complex regional pain syndrome, and painful bladder syndrome. Abnormal acute and sensory responses to cold exposure occurs following the first rounds of treatment with the chemotherapeutic agent oxaliplatin, the most commonly used anti-tumor treatment for colorectal cancers and others; additionally, oxaliplatin precipitates increased sensitivity to menthol, perhaps due to overexpression or activity of TRPM8. Notably, colorectal cancer is the second leading cause of cancer mortality and fourth most common malignant disease. Cold sensitive-paraesthesias and pharyngeal spasm leading to dysphagia occur as a result of oxaliplatin treatment and represent sensory neurotoxicity (sNT) associated with oxaliplatin. Moreover, all platinum agents, including cisplatin, carboplatin and oxaliplatin, used to treat lung carcinoma, testicular cancer and ovarian cancer, are known to induce sensory neuropathy.

Thermal hyperalgesia is a clinical marker of oxaliplatin neurotoxicity at early onset, and may predict severe neuropathy. Cold hypersensitivity is the hallmark of oxaliplatin-induced neuropathy. Pre-clinically, oxaliplatin-induced hypersensitivity to innocuous and noxious cold stimuli proceeds through a TRPM8-dependent mechanism, as confirmed by knockout studies. TRPM8 blockade with the bifunctional TRPM8/TRPV1 antagonist capsazepine dose-dependently inhibits oxaliplatin-induced cold allodynia in vivo, an effect not seen with TRPV1 antagonist 5′-iodoresiniferatoxin. Similarly, TRPM8 mRNA expression in the DRG significantly increases following cold allodynia induced by single injection of oxaliplatin, an effect functionally linked to an increase of response to TRPM8 stimulation in vivo. These studies are in agreement with previous literature reporting similar increases in TRPM8 mRNA expression in L4-L6 DRG following induction of cold hyperalgesia by oxaliplatin treatment, as well as treatment with the oxaliplatin metabolite oxalate. In vitro, TRPM8 mRNA levels were also significantly increased in oxaliplatin-treated DRG neurons. Compound 14 was evaluated for its ability to block sensory responses to cold in a model of peripheral neuropathic pain induced by oxaliplatin, as cold hypersensitivity is the hallmark of oxaliplatin-induced neuropathy.

Currently, no pharmacotherapies are available for CIPN, despite advances in cancer treatment with new and more targeted chemotherapies. Duloxetine, used for diabetic neuropathy, has only a modest analgesic effect in patients with CIPN induced by oxaliplatin.

The activity of Compound 14 was evaluated 7 days after three intraperitoneal injections of OXP (6 mg/kg) in C₅₇/BL6 mice, when cold allodynia had developed. After single subcutaneous administration of Compound 14 (0.1 and 1 μg), attenuated cold allodynia is evident at 15 min and reaches maximum inhibition 30 min after administration (FIG. 11), in a dose dependent manner. This data suggests that Compound 14 may be a viable therapeutic scaffold for the treatment of CIPN.

The antinociceptive activity of Compound 14 surpasses that of another reported, potent TRPM8 antagonist (R)-(−)-10e in the same assay. (R)-(−)-10e inhibits oxaliplatin-induced nocifensive paw licking, though at >10,000-fold higher doses when administered p.o. Similarly, a tryptophan-based TRPM8 antagonist reverses oxaliplatin-induced cold allodynia in C₅₇/BL6 mice at the same doses as Compound 14, however its effects are short lasting, perhaps due to a labile ester group. Compound 14 significantly suppresses OXP effects at 60 minutes, equal to two t_{1/2} values, suggesting that additional phenomena are at hand. For example, Compound 14 may function in vivo via a drug depot effect, imparted by its lipophilicity. Of equal importance, Compound 14 and other menthol-like antagonists reported herein may be useful chemical tools to explore the role of TRPM8 in other pain states such as chronic neuropathic pain in vivo, for which the role of this ion channel is controversial. Such small molecule scaffolds can then provide pharmacotherapies for sensory neuropathies.

In short, probing replacements of the (−)-menthyl ring of Compound 1 with lipophilic moieties, as indicated by the computational studies of TRPM8 antagonist scaffolds, as described herein and carried out in the above-described human TRPM8 homology model, allows for the discovery of a novel series of small molecule ligands with highly potent hTRPM8 antagonist activity in the Ca²⁺ flux assay and >10-fold selectivity vs. TRPV1 and TRPA1. These menthol-optimized analogs can find use as chemical probes to investigate TRPM8 pharmacology, structure-function studies, and structural biology studies, along with menthol-based agonists reported by Dendreon (D-3263 and others) and Proctor & Gamble, with nanomolar agonist activity at human TRPM8. This series may also provide possibilities for pharmacotherapies for CIPN induced by oxaliplatin and other TRPM8-related sensory neuropathies, as Compound 14 is able to attenuate oxaliplatin-induced cold allodynia in vivo. Additionally, these compounds may also aid in the identification of a TRPM8 pharmacophore for the rational design of small molecule ligands and therapeutic scaffolds. One of the most potent analogs identified (Compound 14), has superior antagonist activity in both Ca²⁺ flux and patch-clamp assays, and can dose-dependently inhibit the effects of multiple TRPM8 activators (icilin, menthol and cold) across a battery of in vitro and in vivo assays. Compound 14 is readily accessible synthetically from an amidation reaction, and possesses no chiral centers, thus enhancing its attractiveness. Compound 14 is one of the most potent TRPM8 antagonists described to date. The above SAR studies suggests its spiro[4.5]decan-8-yl moiety is a preferred scaffold for TRPM8 recognition vs. the cognate ligand (−)-menthol, and represents an optimal menthol surrogate. The -field awaits a TRPM8 structure complexed with a menthol-derived antagonist, to address subtleties surrounding channel activation vs. inactivation, particularly in the context of natural product-based scaffolds. The antagonist, Compound 14 represents a viable option for these studies, to parse antagonist-vs. agonist-mediated molecular events at TRPM8, along with menthol analog WS-12.

All publications, patents, and patent applications mentioned in this specification are herein incorporated by reference, including the references set forth in the following list:

### REFERENCES

1. Winchester, W. J.; Gore, K.; Glatt, S.; Petit, W.; Gardiner, J. C.; Conlon, K.; Postlethwaite, M.; Saintot, P. P.; Roberts, S.; Gosset, J. R.; Matsuura, T.; Andrews, M. D.; Glossop, P. A.; Palmer, M. J.; Clear, N.; Collins, S.; Beaumont, K.; Reynolds, D. S. (2014) Inhibition of TRPM8 channels reduces pain in the cold pressor test in humans. J. Pharmacol. Exp. Ther. 351, 259-269.
2. Kobayashi, K.; Fukuoka, T.; Obata, K.; Yamanaka, H.; Dai, Y.; Tokunaga, A.; Noguchi, K. (2005) Distinct expression of TRPM8, TRPA1, and TRPV1 mRNAs in rat primary afferent neurons with adelta/c-fibers and colocalization with trk receptors. J. Comp. Neurol. 493, 596-606.
3. Proudfoot, C. J.; Garry, E. M.; Cottrell, D. F.; Rosie, R.; Anderson, H.; Robertson, D. C.; Fleetwood-Walker, S. M.; Mitchell, R. (2006) Analgesia mediated by the TRPM8 cold receptor in chronic neuropathic pain. Curr. Biol. 16, 1591-1605.
4. McKemy, D. D.; Neuhausser, W. M.; Julius, D. (2002) Identification of a cold receptor reveals a general role for TRP channels in thermosensation. Nature 416, 52-58.
5. Janssens, A.; Voets, T. (2011) Ligand stoichiometry of the cold- and menthol-activated channel TRPM8. J. Physiol. 589, 4827-4835.
6. Perez de Vega, M. J.; Gomez-Monterrey, I.; Ferrer-Montiel, A.; Gonzalez-Muniz, R. (2016) Transient Receptor Potential Melastatin 8 Channel (TRPM8) Modulation: Cool Entryway for Treating Pain and Cancer. J. Med. Chem. 59, 10006-10029.
7. Yin, Y.; Wu, M.; Zubcevic, L.; Borschel, W. F.; Lander, G. C.; Lee, S. Y. (2017) Structure of the cold- and menthol-sensing ion channel TRPM8. Science. 359, 237-241.
8. Yin, Y.; Le, S. C.; Hsu, A. L.; Borgnia, M. J.; Yang, H.; Lee, S. Y. (2019) Structural basis of cooling agent and lipid sensing by the cold-activated TRPM8 channel. Science. 363, eaav9334.
9. Andrews, M. D.; A f Forselles, K.; Beaumont, K.; Galan, S. R.; Glossop, P. A.; Grenie, M.; Jessiman, A.; Kenyon, A. S.; Lunn, G.; Maw, G.; Owen, R. M.; Pryde, D. C.; Roberts, D.; Tran, T. D. (2015) Discovery of a Selective TRPM8 Antagonist with Clinical Efficacy in Cold-Related Pain. ACS Med. Chem. Lett. 6, 419-424.
10. Horne, D. B.; Tamayo, N. A.; Bartberger, M. D.; Bo, Y.; Clarine, J.; Davis, C. D.; Gore, V. K.; Kaller, M. R.; Lehto, S. G.; Ma, V. V.; Nishimura, N.; Nguyen, T. T.; Tang, P.; Wang, W.; Youngblood, B. D.; Zhang, M.; Gavva, N. R.; Monenschein, H.; Norman, M. H. (2014) Optimization of potency and pharmacokinetic properties of tetrahydroisoquinoline transient receptor potential melastatin 8 (TRPM8) antagonists. J. Med. Chem. 57, 2989-3004.
11. Gavva, N. R.; Davis, C.; Lehto, S. G.; Rao, S.; Wang, W.; Zhu, D. X. (2012) Transient receptor potential melastatin 8 (TRPM8) channels are involved in body temperature regulation. Mol. Pain. 8, 36.
12. Lashinger, E. S.; Steiginga, M. S.; Hieble, J. P.; Leon, L. A.; Gardner, S. D.; Nagilla, R.; Davenport, E. A.; Hoffman, B. E.; Laping, N. J.; Su, X. (2008) AMTB, a TRPM8 channel blocker: evidence in rats for activity in overactive bladder and painful bladder syndrome. Am. J. Physiol. Renal Physiol. 295, F803-810.
13. Horne, D. B.; Biswas, K.; Brown, J.; Bartberger, M. D.; Clarine, J.; Davis, C. D.; Gore, V. K.; Harried, S.; Horner, M.; Kaller, M. R.; Lehto, S. G.; Liu, Q.; Ma, V. V.; Monenschein, H.; Nguyen, T. T.; Yuan, C.; Youngblood, B. D.; Zhang, M.; Zhong, W.; Allen, J. R.; Chen, J. J.; Gavva, N. (2018) Discovery of TRPM8 Antagonist (S)-6-(((3-fluoro-4-(trifluoromethoxy)phenyl)(3-fluoropyridin-2-yl)methyl)carbamoyl)nicotinic acid (AMG 333), a Clinical Candidate for the Treatment of Migraine. J. Med. Chem. 61, 8186-8201.
14. De Caro, C.; Russo, R.; Avagliano, C.; Cristiano, C.; Calignano, A.; Aramini, A.; Bianchini, G.; Allegretti, M.; Brandolini, L. (2018) Antinociceptive effect of two novel transient receptor potential melastatin 8 antagonists in acute and chronic pain models in rat. Br. J. Pharmacol. 175, 1691-1706.
15. Almeida, M. C.; Hew-Butler, T.; Soriano, R. N.; Rao, S.; Wang, W.; Wang, J.; Tamayo, N.; Oliveira, D. L.; Nucci, T. B.; Aryal, P.; Garami, A.; Bautista, D.; Gavva, N. R.; Romanovsky, A. A. (2012) Pharmacological blockade of the cold receptor TRPM8 attenuates autonomic and behavioral cold defenses and decreases deep body temperature. J. Neurosci. 32, 2086-2099.
16. Okamoto, Y.; Ohkubo, T.; Ikebe, T.; Yamazaki, J. (2012) Blockade of TRPM8 activity reduces the invasion potential of oral squamous carcinoma cell lines. Int. J Oncol. 40, 1431-1440.
17. Willis, D. N.; Liu, B.; Ha, M. A.; Jordt, S. E.; Morris, J. B. (2011) Menthol attenuates respiratory irritation responses to multiple cigarette smoke irritants. FASEB J. 25, 4434-4444.
18. Lin, A. H.; Liu, M. H.; Ko, H. B.; Perng, D. W.; Lee, T. S.; Kou, Y. R. (2017) Inflammatory Effects of Menthol vs. Non-menthol Cigarette Smoke Extract on Human Lung Epithelial Cells: A Double-Hit on TRPM8 by Reactive Oxygen Species and Menthol. Front. Physiol. 8, 263.
19. Patel, R.; Goncalves, L.; Leveridge, M.; Mack, S. R.; Hendrick, A.; Brice, N. L.; Dickenson, A. H. (2014) Anti-hyperalgesic effects of a novel TRPM8 agonist in neuropathic rats: a comparison with topical menthol. Pain 155, 2097-2107.
20. Voets, T.; Owsianik, G.; Janssens, A.; Talavera, K.; Nilius, B. (2007) TRPM8 voltage sensor mutants reveal a mechanism for integrating thermal and chemical stimuli. Nat. Chem. Biol. 3, 174-182.
21. Bandell, M.; Dubin, A. E.; Petrus, M. J.; Orth, A.; Mathur, J.; Hwang, S. W.; Patapoutian, A. (2006) High-throughput random mutagenesis screen reveals TRPM8 residues specifically required for activation by menthol. Nat. Neurosci. 9, 493-500.
22. Rath, P.; Hilton, J. K.; Sisco, N. J.; Van Horn, W. D. (2016) Implications of Human Transient Receptor Potential Melastatin 8 (TRPM8) Channel Gating from Menthol Binding Studies of the Sensing Domain. Biochemistry 55, 114-124.
23. Chuang, H. H.; Neuhausser, W. M.; Julius, D. (2004) The super-cooling agent icilin reveals a mechanism of coincidence detection by a temperature-sensitive TRP channel. Neuron 43, 859-869.
24. Autzen, H. E.; Myasnikov, A. G.; Campbell, M. G.; Asarnow, D.; Julius, D.; Cheng, Y. (2018) Structure of the human TRPM4 ion channel in a lipid nanodisc. Science 359, 228-232.
25. Matos-Cruz, V.; Schneider, E. R.; Mastrotto, M.; Merriman, D. K.; Bagriantsev, S. N.; Gracheva, E. O. (2017) Molecular Prerequisites for Diminished Cold Sensitivity in Ground Squirrels and Hamsters. Cell. Rep. 21, 3329-3337.
26. Ortar, G.; De Petrocellis, L.; Morera, L.; Moriello, A. S.; Orlando, P.; Morera, E.; Nalli, M.; Di Marzo, V. (2010) (−)-Menthylamine derivatives as potent and selective antagonists of transient receptor potential melastatin type-8 (TRPM8) channels. Bioorg Med. Chem. Lett. 20, 2729-2732.
27. Lampe, T.; Alonso-Alija, C.; Beck, H.; Rosentreter, U.; Sandner, P.; Stahl, E.; Stelte-Ludwig, B. Preparation of substituted 2-benzyloxy-benzoic acid amide derivatives as Cold Menthol Receptor 1 (CMR-1) modulators for treating and preventing urological diseases or disorders. 2007017093 A1.
28. Gavva, N. R.; Klionsky, L.; Qu, Y.; Shi, L.; Tamir, R.; Edenson, S.; Zhang, T. J.; Viswanadhan, V. N.; Toth, A.; Pearce, L. V.; Vanderah, T. W.; Porreca, F.; Blumberg, P. M.; Lile, J.; Sun, Y.; Wild, K.; Louis, J. C.; Treanor, J. J. (2004) Molecular determinants of vanilloid sensitivity in TRPV1. J. Biol. Chem. 279, 20283-20295.
29. Behrendt, H. J.; Germann, T.; Gillen, C.; Hatt, H.; Jostock, R. (2004) Characterization of the mouse cold-menthol receptor TRPM8 and vanilloid receptor type-1 VR1 using a fluorometric imaging plate reader (FLIPR) assay. Br. J. Pharmacol. 141, 737-745.
30. Rami, H. K.; Thompson, M.; Wyman, P.; Jerman, J. C.; Egerton, J.; Brough, S.; Stevens, A. J.; Randall, A. D.; Smart, D.; Gunthorpe, M. J.; Davis, J. B. (2004) Discovery of small molecule antagonists of TRPV1. BioorgMed. Chem. Lett. 14, 3631-3634.
31. Weil, A.; Moore, S. E.; Waite, N. J.; Randall, A.; Gunthorpe, M. J. (2005) Conservation of functional and pharmacological properties in the distantly related temperature sensors TRVP1 and TRPM8. Mol. Pharmacol. 68, 518-527.
32. Knowlton, W. M.; Daniels, R. L.; Palkar, R.; McCoy, D. D.; McKemy, D. D. (2011) Pharmacological blockade of TRPM8 ion channels alters cold and cold pain responses in mice. PLoS One 6, e25894.
33. Andersson, D. A.; Chase, H. W.; Bevan, S. (2004) TRPM8 activation by menthol, icilin, and cold is differentially modulated by intracellular pH. J. Neurosci. 24, 5364-5369.
34. Andersson, D. A.; Nash, M.; Bevan, S. (2007) Modulation of the cold-activated channel TRPM8 by lysophospholipids and polyunsaturated fatty acids. J. Neurosci. 27, 3347-3355.
35. Hilton, J. K.; Salehpour, T.; Sisco, N. J.; Rath, P.; Van Horn, W. D. (2018) Phosphoinositide-interacting regulator of TRP (PIRT) has opposing effects on human and mouse TRPM8 ion channels. J. Biol. Chem. 293, 9423-9434.
36. Bertamino, A.; Ostacolo, C.; Ambrosino, P.; Musella, S.; Di Sarno, V.; Ciaglia, T.; Soldovieri, M. V.; Iraci, N.; Fernandez Carvajal, A.; de la Torre-Martinez, R.; Ferrer-Montiel, A.; Gonzalez Muniz, R.; Novellino, E.; Taglialatela, M.; Campiglia, P.; Gomez-Monterrey, I. (2016) Tryptamine-Based Derivatives as Transient Receptor Potential Melastatin Type 8 (TRPM8) Channel Modulators. J. Med. Chem. 59, 2179-2191.
37. de la Torre-Martinez, R.; Bonache, M. A.; Llabres-Campaner, P. J.; Balsera, B.; Fernandez-Carvajal, A.; Fernandez-Ballester, G.; Ferrer-Montiel, A.; Perez de Vega, M. J.; Gonzalez-Muniz, R. (2017) Synthesis, high-throughput screening and pharmacological characterization of beta-lactam derivatives as TRPM8 antagonists. Sci. Rep. 7, 10766.
38. Bertamino, A.; Iraci, N.; Ostacolo, C.; Ambrosino, P.; Musella, S.; Di Sarno, V.; Ciaglia, T.; Pepe, G.; Sala, M.; Soldovieri, M. V.; Mosca, I.; Gonzalez-Rodriguez, S.; Fernandez-Carvajal, A.; Ferrer-Montiel, A.; Novellino, E.; Taglialatela, M.; Campiglia, P.; Gomez-Monterrey, I. (2018) Identification of a Potent Tryptophan-Based TRPM8 Antagonist With in Vivo Analgesic Activity. J. Med. Chem. 61, 6140-6152.
39. Wang, L.; Fu, T. M.; Zhou, Y.; Xia, S.; Greka, A.; Wu, H. (2018) Structures and gating mechanism of human TRPM2. Science 362, eaav4809.
40. De Petrocellis, L.; Ligresti, A.; Schiano Moriello, A.; Iappelli, M.; Verde, R.; Stott, C. G.; Cristino, L.; Orlando, P.; Di Marzo, V. (2013) Non-THC cannabinoids inhibit prostate carcinoma growth in vitro and in vivo: pro-apoptotic effects and underlying mechanisms. Br. J. Pharmacol. 168, 79-102.
41. Ha, M. A.; Smith, G. J.; Cichocki, J. A.; Fan, L.; Liu, Y. S.; Caceres, A. I.; Jordt, S. E.; Morris, J. B. (2015) Menthol attenuates respiratory irritation and elevates blood cotinine in cigarette smoke exposed mice. PLoS One 10, e0117128.
42. Tamayo, N. A.; Bo, Y.; Gore, V.; Ma, V.; Nishimura, N.; Tang, P.; Deng, H.; Klionsky, L.; Lehto, S. G.; Wang, W.; Youngblood, B.; Chen, J.; Correll, T. L.; Bartberger, M. D.; Gavva, N. R.; Norman, M. H. (2012) Fused piperidines as a novel class of potent and orally available transient receptor potential melastatin type 8 (TRPM8) antagonists. J. Med. Chem. 55, 1593-1611.
43. Ohmi, M.; Shishido, Y.; Inoue, T.; Ando, K.; Fujiuchi, A.; Yamada, A.; Watanabe, S.; Kawamura, K. (2014) Identification of a novel 2-pyridyl-benzensulfonamide derivative, RQ-00203078, as a selective and orally active TRPM8 antagonist. Bioorg. Med. Chem. Lett. 24, 5364-5368.
44. Caceres, A. I.; Liu, B.; Jabba, S. V.; Achanta, S.; Morris, J. B.; Jordt, S. E. (2017) Transient Receptor Potential Cation Channel Subfamily M Member 8 channels mediate the anti-inflammatory effects of eucalyptol. Br. J. Pharmacol. 174, 867-879.
45. Dodziuk, H.; Jaszunski, M.; Schilf, W. (2005) 1H and 13C NMR chemical shifts and spin-spin coupling constants in trans- and cis-decalins. Magn. Reson. Chem. 43, 639-646.
46. Browne, L. M.; Klinck, R. E.; Stothers, J. B. (1979) Carbon-13 NMR Studies: 82.t Carbon-13 spectra of several 10-methyl-cis-decalins. Conformational preferences of some cis-decalin derivatives. Org. Magn. Reson. 12, 561-568.
47. Player, M. R.; Parks, D. J.; Parsons, W.; Meegalla, S. K.; Illig, C. R.; Ballentine, S. K. Heterocyclic benzimidazoles as TRPM8 modulators. US 20100093788 A1.
48. Chaudhari, S. S.; Kadam, A. B.; Khairatkar-Joshi, N.; Mukhopadhyay, I.; Karnik, P. V.; Raghuram, A.; Rao, S. S.; Vaiyapuri, T. S.; Wale, D. P.; Bhosale, V. M.; Gudi, G. S.; Sangana, R. R.; Thomas, A. (2013) Synthesis and pharmacological evaluation of novel N-aryl-3,4-dihydro-1′H-spiro[chromene-2,4′-piperidine]-1′-carboxamides as TRPM8 antagonists. Bioorg. Med. Chem. 21, 6542-6553.
49. Shishido, Y.; Ohmi, M.; Ando, K. Preparation of azaspiro derivatives as TRPM8 antagonists. WO 2015136947 A1.
50. Klein, A. H.; Iodi Carstens, M.; McCluskey, T. S.; Blancher, G.; Simons, C. T.; Slack, J. P.; Furrer, S.; Carstens, E. (2011) Novel menthol-derived cooling compounds activate primary and second-order trigeminal sensory neurons and modulate lingual thermosensitivity. Chem. Senses 36, 649-658.
51. Bodding, M.; Wissenbach, U.; Flockerzi, V. (2007) Characterisation of TRPM8 as a pharmacophore receptor. Cell Calcium 42, 618-628.
52. Zhu, B.; Xia, M.; Xu, X.; Ludovici, D. W.; Tennakoon, M.; Youngman, M. A.; Matthews, J. M.; Dax, S. L.; Colburn, R. W.; Qin, N.; Hutchinson, T. L.; Lubin, M. L.; Brandt, M. R.; Stone, D. J.; Flores, C. M.; Macielag, M. J. (2013) Arylglycine derivatives as potent transient receptor potential melastatin 8 (TRPM8) antagonists. Bioorg. Med. Chem. Lett. 23, 2234-2237.
53. Calvo, R. R.; Meegalla, S. K.; Parks, D. J.; Parsons, W. H.; Ballentine, S. K.; Lubin, M. L.; Schneider, C.; Colburn, R. W.; Flores, C. M.; Player, M. R. (2012) Discovery of vinylcycloalkyl-substituted benzimidazole TRPM8 antagonists effective in the treatment of cold allodynia. Bioorg. Med. Chem. Lett. 22, 1903-1907.
54. Patel, R.; Goncalves, L.; Newman, R.; Jiang, F. L.; Goldby, A.; Reeve, J.; Hendrick, A.; Teall, M.; Hannah, D.; Almond, S.; Brice, N.; Dickenson, A. H. (2014) Novel TRPM8 antagonist attenuates cold hypersensitivity after peripheral nerve injury in rats. J. Pharmacol. Exp. Ther. 349, 47-55.
55. Takaishi, M.; Uchida, K.; Suzuki, Y.; Matsui, H.; Shimada, T.; Fujita, F.; Tominaga, M. (2016) Reciprocal effects of capsaicin and menthol on thermosensation through regulated activities of TRPV1 and TRPM8. J. Physiol. Sci. 66, 143-155.
56. Macpherson, L. J.; Hwang, S. W.; Miyamoto, T.; Dubin, A. E.; Patapoutian, A.; Story, G. M. (2006) More than cool: promiscuous relationships of menthol and other sensory compounds. Mol. Cell Neurosci. 32, 335-343.
57. Karashima, Y.; Damann, N.; Prenen, J.; Talavera, K.; Segal, A.; Voets, T.; Nilius, B. (2007) Bimodal action of menthol on the transient receptor potential channel TRPA1. J. Neurosci. 27, 9874-9884.
58. Story, G. M.; Peier, A. M.; Reeve, A. J.; Eid, S. R.; Mosbacher, J.; Hricik, T. R.; Earley, T. J.; Hergarden, A. C.; Andersson, D. A.; Hwang, S. W.; McIntyre, P.; Jegla, T.; Bevan, S.; Patapoutian, A. (2003) ANKTM1, a TRP-like channel expressed in nociceptive neurons, is activated by cold temperatures. Cell 112, 819-829.
59. Karashima, Y.; Talavera, K.; Everaerts, W.; Janssens, A.; Kwan, K. Y.; Vennekens, R.; Nilius, B.; Voets, T. (2009) TRPA1 acts as a cold sensor in vitro and in vivo. Proc. Natl. Acad. Sci. USA 106, 1273-1278.
60. McNamara, C. R.; Mandel-Brehm, J.; Bautista, D. M.; Siemens, J.; Deranian, K. L.; Zhao, M.; Hayward, N. J.; Chong, J. A.; Julius, D.; Moran, M. M.; Fanger, C. M. (2007) TRPA1 mediates formalin-induced pain. Proc. Natl. Acad. Sci. USA 104, 13525-13530.
61. Klionsky, L.; Tamir, R.; Gao, B.; Wang, W.; Immke, D. C.; Nishimura, N.; Gavva, N. R. (2007) Species-specific pharmacology of Trichloro(sulfanyl)ethyl benzamides as transient receptor potential ankyrin 1 (TRPA1) antagonists. Mol. Pain 3, 39.
62. El Kouhen, R.; Surowy, C. S.; Bianchi, B. R.; Neelands, T. R.; McDonald, H. A.; Niforatos, W.; Gomtsyan, A.; Lee, C. H.; Honore, P.; Sullivan, J. P.; Jarvis, M. F.; Faltynek, C. R. (2005) A-425619 [1-isoquinolin-5-yl-3-(4-trifluoromethyl-benzyl)-urea], a novel and selective transient receptor potential type V1 receptor antagonist, blocks channel activation by vanilloids, heat, and acid. J. Pharmacol. Exp. Ther. 314, 400-409.
63. Diver, M. M.; Cheng, Y.; Julius, D. (2019) Structural insights into TRPM8 inhibition and desensitization. Science 365, 1434-1440.
64. Gao, Y.; Cao, E.; Julius, D.; Cheng, Y. (2016) TRPV1 structures in nanodiscs reveal mechanisms of ligand and lipid action. Nature 534, 347-351.
65. Paulsen, C. E.; Armache, J. P.; Gao, Y.; Cheng, Y.; Julius, D. (2015) Structure of the TRPA1 ion channel suggests regulatory mechanisms. Nature 525, 552.
66. Wei, E. T. (1983) Inhibition of shaking movements in rats by central administration of cholinergic and adrenergic agents. Psychopharmacology (Berl) 81, 111-114.
67. Wei, E. T.; Seid, D. A. (1983) AG-3-5: a chemical producing sensations of cold. J. Pharm. Pharmacol. 35, 110-112.
68. Colburn, R. W.; Lubin, M. L.; Stone, D. J., Jr.; Wang, Y.; Lawrence, D.; D′Andrea, M. R.; Brandt, M. R.; Liu, Y.; Flores, C. M.; Qin, N. (2007) Attenuated cold sensitivity in TRPM8 null mice. Neuron 54, 379-386.
69. Kono, T.; Satomi, M.; Suno, M.; Kimura, N.; Yamazaki, H.; Furukawa, H.; Matsubara, K. (2012) Oxaliplatin-induced neurotoxicity involves TRPM8 in the mechanism of acute hypersensitivity to cold sensation. Brain Behav. 2, 68-73.
70. Ling, B.; Authier, N.; Balayssac, D.; Eschalier, A.; Coudore, F. (2007) Behavioral and pharmacological description of oxaliplatin-induced painful neuropathy in rat. Pain 128, 225-234. 71. Mizoguchi, S.; Andoh, T.; Yakura, T.; Kuraishi, Y. (2016) Involvement of c-Myc-mediated transient receptor potential melastati 8 expression in oxaliplatin-induced cold allodynia in mice. Pharmacol. Rep. 68, 645-648.
72. Deuis, J. R.; Dvorakova, L. S.; Vetter, I. (2017) Methods Used to Evaluate Pain Behaviors in Rodents. Front. Mol. Neurosci. 10, 284.
73. Naziroglu, M.; Braidy, N. (2017) Thermo-Sensitive TRP Channels: Novel Targets for Treating Chemotherapy-Induced Peripheral Pain. Front. Physiol. 8, 1040.
74. Jensen, T. S.; Finnerup, N. B. (2014) Allodynia and hyperalgesia in neuropathic pain: clinical manifestations and mechanisms. Lancet Neurol. 13, 924-935.
75. Binder, A.; Stengel, M.; Maag, R.; Wasner, G.; Schoch, R.; Moosig, F.; Schommer, B.; Baron, R. (2007) Pain in oxaliplatin-induced neuropathy--sensitisation in the peripheral and central nociceptive system. Eur. J Cancer. 43, 2658-2663.
76. Attal, N.; Bouhassira, D.; Gautron, M.; Vaillant, J. N.; Mitry, E.; Lepere, C.; Rougier, P.; Guirimand, F. (2009) Thermal hyperalgesia as a marker of oxaliplatin neurotoxicity: a prospective quantified sensory assessment study. Pain 144, 245-252.
77. Grothey, A. (2003) Oxaliplatin-safety profile: neurotoxicity. Semin. Oncol. 30, 5-13.
78. Kelland, L. (2007) The resurgence of platinum-based cancer chemotherapy. Nat. Rev. Cancer 7, 573-584.
79. Descoeur, J.; Pereira, V.; Pizzoccaro, A.; Francois, A.; Ling, B.; Maffre, V.; Couette, B.; Busserolles, J.; Courteix, C.; Noel, J.; Lazdunski, M.; Eschalier, A.; Authier, N.; Bourinet, E. (2011) Oxaliplatin-induced cold hypersensitivity is due to remodelling of ion channel expression in nociceptors. EMBO Mol. Med. 3, 266-278.
80. Gauchan, P.; Andoh, T.; Kato, A.; Kuraishi, Y. (2009) Involvement of increased expression of transient receptor potential melastatin 8 in oxaliplatin-induced cold allodynia in mice. Neurosci. Lett. 458, 93-95.
81. Kawashiri, T.; Egashira, N.; Kurobe, K.; Tsutsumi, K.; Yamashita, Y.; Ushio, S.; Yano, T.; Oishi, R. (2012) L type Ca(2)+ channel blockers prevent oxaliplatin-induced cold hyperalgesia and TRPM8 overexpression in rats. Mol. Pain 8, 7.
82. Ta, L. E.; Bieber, A. J.; Carlton, S. M.; Loprinzi, C. L.; Low, P. A.; Windebank, A. J. (2010) Transient Receptor Potential Vanilloid 1 is essential for cisplatin-induced heat hyperalgesia in mice. Mol. Pain 6, 15.
83. Staff, N. P.; Grisold, A.; Grisold, W.; Windebank, A. J. (2017) Chemotherapy-induced peripheral neuropathy: A current review. Ann. Neurol. 81, 772-781.
84. Shapiro, C. L. (2016) Highlights of Recent Findings on Quality-of-Life Management for Patients With Cancer and Their Survivors. JAMA Oncol. 2, 1401-1402.
85. Moran, M. M.; Szallasi, A. (2018) Targeting nociceptive transient receptor potential channels to treat chronic pain: current state of the field. Br. J. Pharmacol. 175, 2185-2203.
86. Yelm, K. E.; Wos, J. A.; Bunke, G. M.; Frederick, H.; Haught, J. C.; Hoke, S. H.; Sreekrishna, K. T.; Lin, Y. Synthesis of aryl cyclohexane carboxamide derivatives useful as sensates in consumer products. 20170057911 A1.
87. Wos, J. A.; Yelm, K. E.; Bunke, G. M.; Frederick, H. A.; Reilly, M.; Haught, J. C.; Sreekrishna, K. T.; Lin, Y. Synthesis of cyclohexane ester derivatives useful as cooling sensates in consumer products. 2017106279 A1.
88. Moreno, O.; Natarajan, S.; Duncan, D. F. Small-molecule modulators of Trp-p8 activity. 20080255185 A1.
89. SYBYL-X; 1.3 Tripos International, 1699 South Hanley Rd. St. Louis, Mo., 63144, USA., 2010.
90. Xie, X. Q.; Chen, J. Z.; Billings, E. M. (2003) 3D structural model of the G-protein-coupled cannabinoid CB2 receptor. Proteins 53, 307-319.
91. Marti-Renom, M. A.; Stuart, A. C.; Fiser, A.; Sanchez, R.; Melo, F.; Sali, A. (2000) Comparative protein structure modeling of genes and genomes. Annu. Rev. Biophys. Biomol. Struct. 29, 291-325.
92. Chen, J. Z.; Wang, J.; Xie, X. Q. (2007) GPCR structure-based virtual screening approach for CB2 antagonist search. J. Chem. Inf Model. 47, 1626-1637.
93. Feng, Z.; Algarni, M. H.; Yang, P.; Tong, Q.; Chowdhury, A.; Wang, L.; Xie, X. Q. (2014) Modeling, molecular dynamics simulation, and mutation validation for structure of cannabinoid receptor 2 based on known crystal structures of GPCRs. J. Chem Inf Model. 54, 2483-2499.
94. Teramachi, J.; Silbermann, R.; Yang, P.; Zhao, W.; Mohammad, K. S.; Guo, J.; Anderson, J. L.; Zhou, D.; Feng, R.; Myint, K. Z.; Maertz, N.; Beumer, J. H.; Eiseman, J. L.; Windle, J. J.; Xie, X. Q.; Roodman, G. D.; Kurihara, N. (2016) Blocking the ZZ domain of sequestosomel/p62 suppresses myeloma growth and osteoclast formation in vitro and induces dramatic bone formation in myeloma-bearing bones in vivo. Leukemia 30, 390-398.
95. Feng, Z.; Pearce, L. V.; Xu, X.; Yang, X.; Yang, P.; Blumberg, P. M.; Xie, X. Q. (2015) Structural insight into tetrameric hTRPV1 from homology modeling, molecular docking, molecular dynamics simulation, virtual screening, and bioassay validations. J. Chem. Inf Model. 55, 572-588.
96. Wiederstein, M.; Sippl, M. J. (2007) ProSA-web: interactive web service for the recognition of errors in three-dimensional structures of proteins. Nucleic Acids Res. 35, W407-410.
97. Laskowski, R. A.; MacArthur, M. W.; Moss, D. S.; Thornton, J. M. (1993) PROCHECK: a program to check the stereochemical quality of protein structures. Journal of Applied Crystallography 26, 283-291.
98. Jain, A. N. (1996) Scoring noncovalent protein-ligand interactions: a continuous differentiable function tuned to compute binding affinities. J. Comput. AidedMol. Des. 10, 427-440.
99. Maier, J. A.; Martinez, C.; Kasavajhala, K.; Wickstrom, L.; Hauser, K. E.; Simmerling, C. (2015) ffl4SB: Improving the Accuracy of Protein Side Chain and Backbone Parameters from ff99SB. J. Chem. Theory Comput. 11, 3696-3713.
100. Dickson, C. J.; Madej, B. D.; Skjevik, A. A.; Betz, R. M.; Teigen, K.; Gould, I. R.; Walker, R. C. (2014) Lipidl4: The Amber Lipid Force Field. J. Chem. Theory Comput. 10, 865-879.
101. Jorgensen, W. L.; Chandrasekhar, J.; Madura, J. D.; Impey, R. W.; Klein, M. L. (1983) Comparison of simple potential functions for simulating liquid water. The Journal of Chemical Physics 79, 926-935.
102. Gotz, A. W.; Williamson, M. J.; Xu, D.; Poole, D.; Le Grand, S.; Walker, R. C. (2012) Routine Microsecond Molecular Dynamics Simulations with AMBER on GPUs. 1. Generalized Born. J.. Chem. Theory Comput. 8, 1542-1555.
103. Salomon-Ferrer, R.; Gotz, A. W.; Poole, D.; Le Grand, S.; Walker, R. C. (2013) Routine Microsecond Molecular Dynamics Simulations with AMBER on GPUs. 2. Explicit Solvent Particle Mesh Ewald. J. Chem. Theory Comput. 9, 3878-3888.
104. Case, D.; Cerutti, D.; Cheatham, T.; Darden, T.; Duke, R.; Giese, T.; Gohlke, H.; Goetz, A.; Greene, D.; Homeyer, N. AMBER 16, University of Calfornia: San Francisco, 2016.
105. Loncharich, R. J.; Brooks, B. R.; Pastor, R. W. (1992) Langevin dynamics of peptides: the frictional dependence of isomerization rates of N-acetylalanyl-N-methylamide. Biopolymers 32, 523-535.
106. Izaguirre, J. A.; Catarello, D. P.; Wozniak, J. M.; Skeel, R. D. (2001) Langevin stabilization of molecular dynamics. The Journal of Chemical Physics 114, 2090-2098.
107. Darden, T.; York, D.; Pedersen, L. (1993) Particle mesh Ewald: An N-log(N) method for Ewald sums in large systems. The Journal of ChemicalPhysics 98, 10089-10092.
108. Essmann, U.; Perera, L.; Berkowitz, M. L.; Darden, T.; Lee, H.; Pedersen, L. G. (1995) A smooth particle mesh Ewald method. The Journal of Chemical Physics 103, 8577-8593.
109. Ryckaert, J. P.; Ciccotti, G.; Berendsen, H. J. C. (1977) Numerical integration of the cartesian equations of motion of a system with constraints: molecular dynamics of n-alkanes. Journal of Computational Physics 23, 327-341.
110. Yang, P.; Wang, L.; Xie, X. Q. (2012) Latest advances in novel cannabinoid CB(2) ligands for drug abuse and their therapeutic potential. Future Med. Chem. 4, 187-204.
111. Hawkins, G. D.; Cramer, C. J.; Truhlar, D. G. (1996) Parametrized Models of Aqueous Free Energies of Solvation Based on Pairwise Descreening of Solute Atomic Charges from a Dielectric Medium. The Journal ofPhysical Chemistry 100, 19824-19839.
112. Kollman, P. A.; Massova, I.; Reyes, C.; Kuhn, B.; Huo, S.; Chong, L.; Lee, M.; Lee, T.; Duan, Y.; Wang, W.; Donini, O.; Cieplak, P.; Srinivasan, J.; Case, D. A.; Cheatham, T. E., 3rd (2000) Calculating structures and free energies of complex molecules: combining molecular mechanics and continuum models. Acc. Chem. Res. 33, 889-897.
113. Tsui, V.; Case, D. A. (2000) Theory and applications of the generalized Born solvation model in macromolecular simulations. Biopolymers 56, 275-291.
114. Bashford, D.; Case, D. A. (2000) Generalized born models of macromolecular solvation effects. Annu. Rev. Phys. Chem. 51, 129-152.
115. Sitkoff, D.; Sharp, K. A.; Honig, B. (1994) Accurate Calculation of Hydration Free Energies Using Macroscopic Solvent Models. The Journal ofPhysical Chemistry 98 1978-1988.
116. Still, W. C.; Tempczyk, A.; Hawley, R. C.; Hendrickson, T. (1990) Semianalytical treatment of solvation for molecular mechanics and dynamics. Journal of the American Chemical Society 112, 6127-6129.
117. Weiser, J.; Shenkin, P. S.; Still, W. C. (1999) Approximate atomic surfaces from linear combinations of pairwise overlaps (LCPO). Journal of Computational Chemistry 20, 217-230.
118. Hu, J.; Feng, Z.; Ma, S.; Zhang, Y.; Tong, Q.; Alqarni, M. H.; Gou, X.; Xie, X. Q. (2016) Difference and Influence of Inactive and Active States of Cannabinoid Receptor Subtype CB2: From Conformation to Drug Discovery. J. Chem. Inf. Model. 56, 1152-1163.
It will be understood that various details of the presently disclosed subject matter can be changed without departing from the scope of the subject matter disclosed herein. Furthermore, the foregoing description is for the purpose of illustration only, and not for the purpose of limitation.

1. A TRPM8 antagonist, comprising the formula (I): 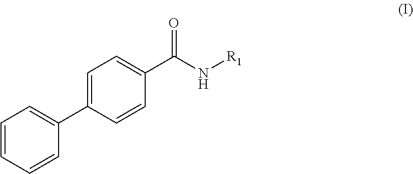
wherein R₁ is selected from a cycloalkyl, a bicycloalkyl, or a tricycloalkyl group, each of which having 7 to 12 carbon atoms, each of which optionally substituted with an alkyl group having 1 to 5 carbons atoms or with a cycloalkyl group having 4 to 12 carbon atoms, and each of which optionally saturated or partially unsaturated.

2. The TRPM8 antagonist of claim 1, wherein R₁ is a bicycloalkyl group, and wherein the bicycloalkyl group is a fused, bridged, or spiro-connected bicycloalkyl group.

3. The TRPM8 antagonist of claim 1, wherein R₁ is a cycloalkyl group, and wherein the cycloalkyl group is a branched or substituted cycloalkyl group.

4. The TRPM8 antagonist of claim 1, wherein R₁ is selected from the group consisting of 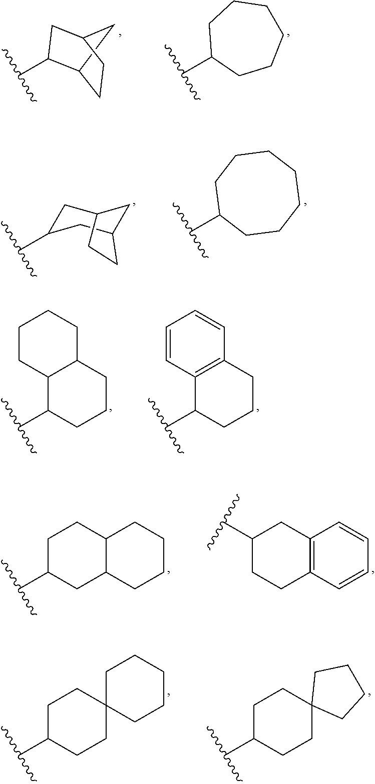
and analogs thereof.

5. The TRPM8 antagonist of claim 1, wherein R₁ is selected from the group consisting of 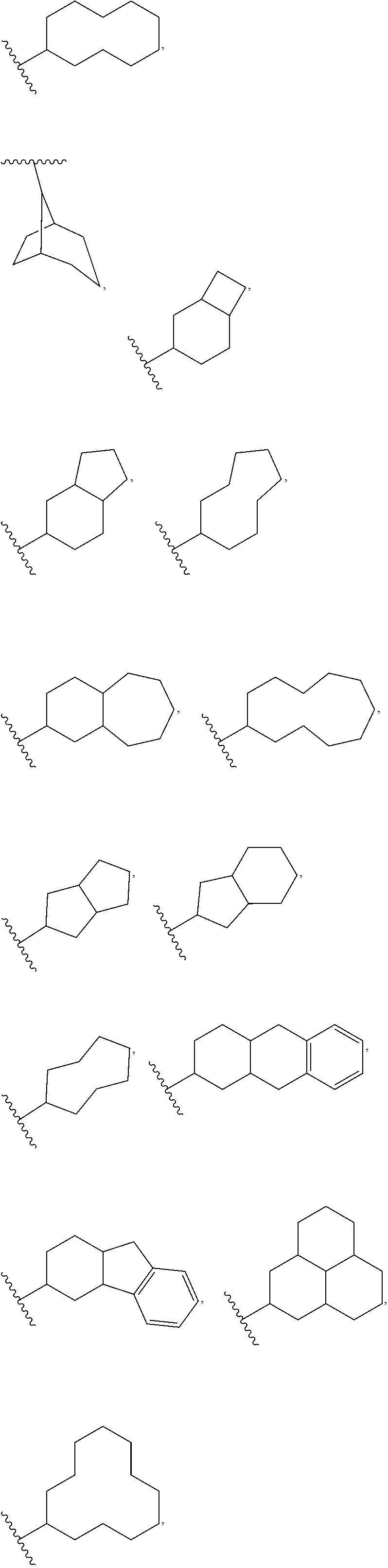
and analogs thereof.

6. The TRPM8 antagonist of claim 1, wherein R₁ is a bicycloalkyl or tricycloalkyl group selected from the group consisting of: 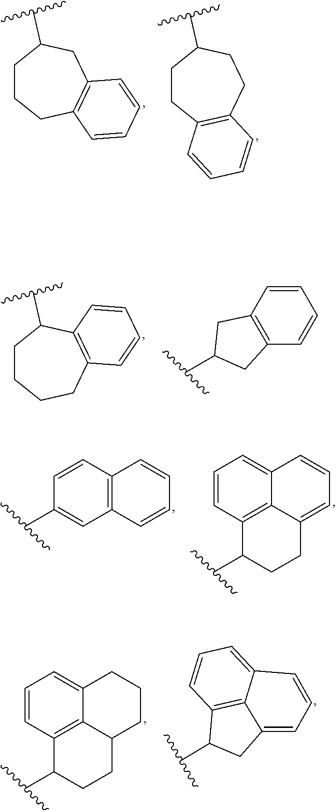
and analogs thereof.

7. (canceled)

8. The TRPM8 antagonist of claim 1, wherein R₁ is a spiro-connected bicycloalkyl group selected from the group consisting of: 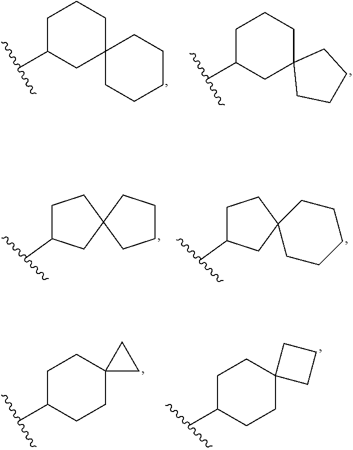
and analogs thereof.

9. The TRPM8 antagonist of claim 1, wherein R₁ is a cycloalkyl group selected from the group consisting of: 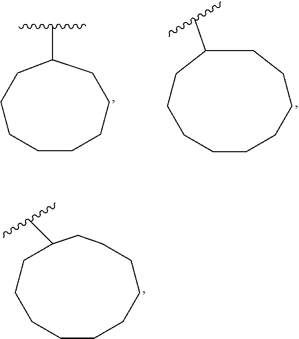 [SMILES: *C1CCCCCCCC1 ; *C1CCCCCCCCC1 ; *C1CCCCCCCCCC1]
and analogs thereof.

10. The TRPM8 antagonist of claim 1, wherein R₁ is a bicycloalkyl or tricycloalkyl group selected from the group consisting of: 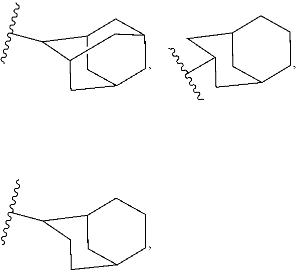
and analogs thereof.

11. The TRPM8 antagonist of claim 4, wherein R₁ is selected from the group consisting of

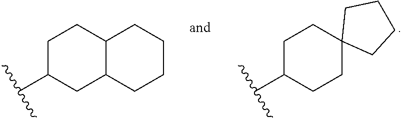 [SMILES: *C1CCC2CCCCC2C1 ; *C1CCC2(CCCC2)CC1]

12. (canceled)

13. (canceled)

14. The TRPM8 antagonist of claim 4, wherein the TRPM8 antagonist has the following formula (V):

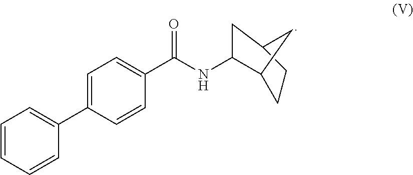 [SMILES: O=C(NC1CC2CCC1C2)c1ccc(-c2ccccc2)cc1]

15. The TRPM8 antagonist of claim 4, wherein the TRPM8 antagonist has the following formula (VI):

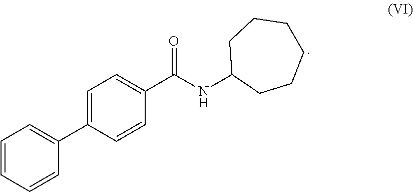 [SMILES: O=C(NC1CCCCCC1)c1ccc(-c2ccccc2)cc1]

16. The TRPM8 antagonist of claim 4, wherein the TRPM8 antagonist has the following formula (VII):

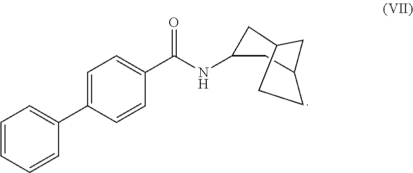 [SMILES: O=C(NC1CC2CCC(C2)C1)c1ccc(-c2ccccc2)cc1]

17. The TRPM8 antagonist of claim 4, wherein the TRPM8 antagonist has the following formula (VIII):

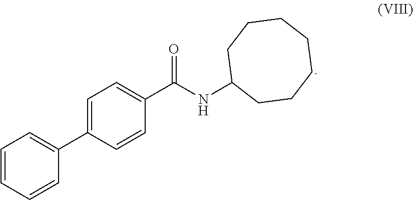 [SMILES: O=C(NC1CCCCCCC1)c1ccc(-c2ccccc2)cc1]

18. The TRPM8 antagonist of claim 4, wherein the TRPM8 antagonist has the following formula (IX):

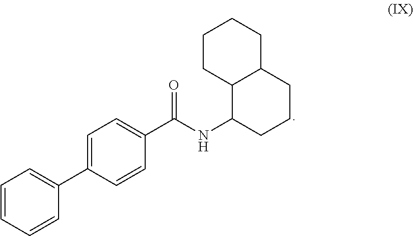 [SMILES: O=C(NC1CCCC2CCCCC21)c1ccc(-c2ccccc2)cc1]

19. The TRPM8 antagonist of claim 4, wherein the TRPM8 antagonist has the following formula (X):

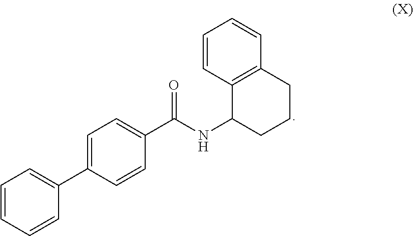 [SMILES: O=C(NC1CCCc2ccccc21)c1ccc(-c2ccccc2)cc1]

20. The TRPM8 antagonist of claim 4, wherein the TRPM8 antagonist has the following formula (XI):

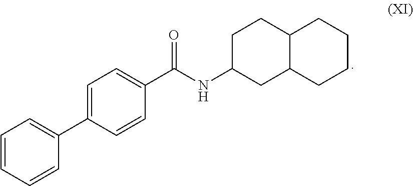 [SMILES: O=C(NC1CCC2CCCCC2C1)c1ccc(-c2ccccc2)cc1]

21. The TRPM8 antagonist of claim 4, wherein the TRPM8 antagonist has the following formula (XII):

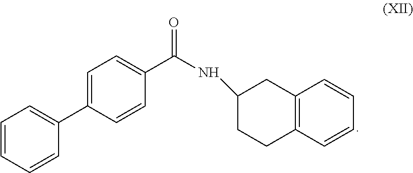 [SMILES: O=C(NC1CCc2ccccc2C1)c1ccc(-c2ccccc2)cc1]

22. (canceled)

23. The TRPM8 antagonist of claim 4, wherein the TRPM8 antagonist has the following formula (XIV):

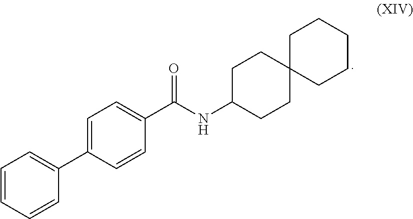 [SMILES: O=C(NC1CCC2(CCCCC2)CC1)c1ccc(-c2ccccc2)cc1]

24. The TRPM8 antagonist of claim 4, wherein the TRPM8 antagonist has the following formula (XV):

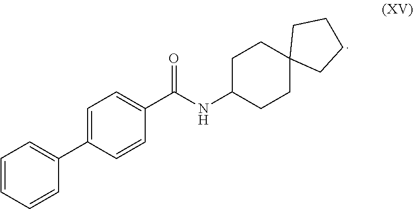 [SMILES: O=C(NC1CCC2(CCCC2)CC1)c1ccc(-c2ccccc2)cc1]

25. A pharmaceutical composition, comprising a TRPM8 antagonist according to claim 1 and a pharmaceutically-acceptable vehicle, carrier, or excipient.

26. A method for treating pain, comprising administering to a subject in need thereof an effective amount of a TRPM8 antagonist according to claim 1.

27.-49. (canceled)

50. The method of claim 26, wherein the pain is neuropathic pain.

51. The method of claim 50, wherein the pain is allodynia.

52. The method of claim 50, wherein the pain is chronic neuropathic pain.

53. The method of claim 50, wherein the pain is chemotherapy-induced neuropathic pain.

54. The method of claim 26, wherein administering the TRPM8 antagonist comprises intravenously, intraperitoneally, intramuscularly, or subcutaneously injecting the TRPM8 antagonist.

55. The method of claim 26, wherein the subject is a human.
